SEQUENCE LISTING (24 sequences)

SEQ ID NO: 1 - DNA, 15 bp, synthetic construct
  source 1..15: mol_type = other DNA
atcagcgggatcttc

SEQ ID NO: 2 - DNA, 15 bp, synthetic construct
  source 1..15: mol_type = other DNA
cgttttcgcaggttg

SEQ ID NO: 3 - DNA, 15 bp, synthetic construct
  source 1..15: mol_type = other DNA
gttttcgcaggttga

SEQ ID NO: 4 - DNA, 15 bp, synthetic construct
  source 1..15: mol_type = other DNA
aagtcacggcacagc

SEQ ID NO: 5 - DNA, 15 bp, synthetic construct
  source 1..15: mol_type = other DNA
agtcacggcacagca

SEQ ID NO: 6 - DNA, 15 bp, synthetic construct
  source 1..15: mol_type = other DNA
catcacgaagcatgt

SEQ ID NO: 7 - DNA, 15 bp, synthetic construct
  source 1..15: mol_type = other DNA
acttaccgttaaata

SEQ ID NO: 8 - DNA, 15 bp, synthetic construct
  source 1..15: mol_type = other DNA
cttaccgttaaatag

SEQ ID NO: 9 - DNA, 15 bp, synthetic construct
  source 1..15: mol_type = other DNA
attttcgtcgtcaag

SEQ ID NO: 10 - DNA, 15 bp, synthetic construct
  source 1..15: mol_type = other DNA
aacattcgatagtac

SEQ ID NO: 11 - DNA, 15 bp, synthetic construct
  source 1..15: mol_type = other DNA
ctctcccgacaaatg

SEQ ID NO: 12 - DNA, 15 bp, synthetic construct
  source 1..15: mol_type = other DNA
tttctccggaataat

SEQ ID NO: 13 - DNA, 15 bp, synthetic construct
  source 1..15: mol_type = other DNA
ttctccggaataatg

SEQ ID NO: 14 - DNA, 15 bp, synthetic construct
  source 1..15: mol_type = other DNA
tgtccccgggagatc

SEQ ID NO: 15 - DNA, 15 bp, synthetic construct
  source 1..15: mol_type = other DNA
cgtaacgcatctgaa

SEQ ID NO: 16 - DNA, 15 bp, synthetic construct
  source 1..15: mol_type = other DNA
gagatcgtatgtcaa

SEQ ID NO: 17 - DNA, 15 bp, synthetic construct
  source 1..15: mol_type = other DNA
accagcgggtgtaga

SEQ ID NO: 18 - DNA, 15 bp, synthetic construct
  source 1..15: mol_type = other DNA
actgccggataagat

SEQ ID NO: 19 - DNA, 15 bp, synthetic construct
  source 1..15: mol_type = other DNA
gataccgaagatcta

SEQ ID NO: 20 - DNA, 16 bp, synthetic construct
  source 1..16: mol_type = other DNA
tttctccgggataatt

SEQ ID NO: 21 - DNA, 15 bp, synthetic construct
  source 1..15: mol_type = other DNA
actaccgaatataaa

SEQ ID NO: 22 - DNA, 15 bp, synthetic construct
  source 1..15: mol_type = other DNA
caaaaacgcagttat

SEQ ID NO: 23 - DNA, 15 bp, synthetic construct
  source 1..15: mol_type = other DNA
aaaaacgcagttatg

SEQ ID NO: 24 - DNA, 15 bp, synthetic construct
  source 1..15: mol_type = other DNA
aaaaaccgaatatca

(19) United States
(12) Patent Application Publication
(10) Pub. No.: US 2025/0210131 A1
(43) Pub. Date: Jun. 26, 2025

(54) ANALYSIS OF GENOMIC WORD FRAMEWORKS ON GENOMIC METHYLATION DATA

(71) Applicant: THE PENN STATE RESEARCH FOUNDATION, University Park, PA (US)

(72) Inventors: Robersy Sanchez Rodriguez, University Park, PA (US); Sally Mackenzie, University Park, PA (US)

(21) Appl. No.: 18/849,885

(22) PCT Filed: Mar. 24, 2023
(86) PCT No.: PCT/US2023/064913

§ 371 Date: Sep. 23, 2024

Related U.S. Application Data
(60) Provisional application No. 63/323,690, filed on Mar. 25, 2022.

(51) Int. Cl. G16B 20/00; G16B 5/00; G16B 30/10

(52) U.S. Cl. CPC G16B 20/00; G16B 5/00; G16B 30/10

(57) ABSTRACT

Genomic-word-frameworks (GWF) analysis of DNA methylation involves analysis of methylation motifs and digital signal processing. GWFs are stretches of DNA sequence covering differentially methylated positions (DMPs). This analysis permits the identification of DNA sequence methylation motifs found in genes with potential epigenetic regulatory functionalities, such as those induced by environmental changes or disease. The analytical heuristic can be implemented and used to identify DNA sequences of methylation motifs with high order DNA base interdependence with respect to methylated cytosines and a base distribution that is statistically nonrandom. These findings set the basis for further model prediction. For example, such model prediction can be used for identifying and treating patients of autism, cancer, and other diseases that benefit from early diagnostics.

### CROSS REFERENCE TO RELATED APPLICATIONS

This application claims priority under 35 U.S.C. § 119 to provisional patent application U.S. Ser. No. 63/323,690, filed Mar. 25, 2022. The provisional patent application is herein incorporated by reference in its entirety, including without limitation, the specification, claims, and abstract, as well as any figures, tables, appendices, or drawings thereof.

### STATEMENT REGARDING FEDERALLY SPONSORED RESEARCH OR DEVELOPMENT

This invention was made with government support under Grant No. GM134056 awarded by the National Institutes of Health. The Government has certain rights in the invention.

### TECHNICAL FIELD

The present disclosure relates generally to improvements in computer science having applications in any industry that can benefit from the study of genes, phenotypes, and/or DNA/RNA. More particularly, but not exclusively, the present disclosure relates to genomic-word-framework analysis of genomic methylation data.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

DNA sequences carry not only information for how to build proteins, but also the regulatory information for living organisms to survive and reproduce, which involves but is not limited to epigenetic information that controls chromatin behavior.

Many diseases and the presence of many phenotypes are not well understood. This is because genes' phenotypic penetrance and expressivity vary due to the different combinations of modifying alleles that are present in one genetic background versus another.

Thus, there exists a need in the art for an apparatus which addresses the potential for superpositioning of more than one structured language throughout the nucleotide sequence of a genome.

### SUMMARY

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present disclosure to improve on or overcome the deficiencies in the art.

It is a further object, feature, and/or advantage of the present disclosure to provide an extension for a general purpose programming language or a statistical programming language. In an embodiment, said extension comprises algorithm(s) that analyze(s) methylation signals on stretches of DNA sequences. In an embodiment, the DNA sequences are characterized by (i) methylation information and (ii) physicochemical information around each methylated cytosine. In an embodiment, the algorithm(s) include one or more functions that can estimate a distance matrix on a set of selected regions of said DNA sequences; analyze a hierarchical cluster on the set of selected of regions; group the set of selected regions into a specified number of clusters; and align multiple DNA sequences from the clusters into methylation motifs.

In some embodiments, the extension is written in the R statistical language.

It is a further object, feature, and/or advantage of the present disclosure to reduce computation load. For example, differentially methylated genes (DMGs) identified with methylation analysis can be integrated to gene networks to identify network hubs via protein-protein interactions network analysis and weighted correlation network analyses. Weighted correlation network analyses based on previous knowledge of DMPs and DMGs has no precedent so far.

It is a further object, feature, and/or advantage of the present disclosure to provide a computerized heuristic. In some embodiments, the computerized heuristic comprises a high order DNA base interdependence with respect to methylated cytosines; and a base distribution that is statistically nonrandom.

According to some aspects of the present disclosure, the heuristic can comprise (1) a statistic (sum, mean, or density, etc.) of an information divergence (ID) estimated for each gene carrying at least one DMP on it (on gene-body or on promoter region); (2) principal component analysis (PCA) wherein the first k-th components carrying 1% or more of the whole sample variance are considered in the downstream analysis; (3) computation of a correlation matrix carrying the pairwise gene correlation, represented as vectors of PCs; (4) analysis of correlation matrix for a network; and (5) contribution of each gene to the discrimination of phenotypes evaluated in terms of the fraction of a cumulative variance from a whole sample variance carried by the gene.

According to some additional aspects of the present disclosure, wherein the ID is selected from the group consisting of: Hellinger divergence/distance, J divergence, total variation distance, etc.

According to some additional aspects of the present disclosure, the PCA can be applied with a pcaLDA function described in the '986 Patent. As a result, genes are represented as k-dimensional vectors of PCs, where the square of each coordinate carries the vector contribution (in terms of variance) to the treatment discrimination from the control group. A correlation matrix can mathematically equivalent (in terms of information) to a weighted correlation network (WCN).

According to some additional aspects of the present disclosure, the WCN is analyzed as was done for the network, which can be a PPI network. New knowledge retrieved from the WCN derived from the raw methylation data and it does not depend on our believe or biological knowledge about the genes presented in the network. Results from the WCN and the PPI network are compared to identified consistent relationships and epigenetic gene contributions to the phenotypes.

According to some additional aspects of the present disclosure, the heuristic further comprises a magnitude is computed as the Euclidean Norm of the gene represented as a vector of k PCs.

It is still yet a further object, feature, and/or advantage of the present disclosure to selectively build motif libraries. For example, methylation motifs can be identified in all DMGs, which provide the raw material to build motif libraries. These libraries can then serve as the fundamental dataset needed to build predictive models with applications in plant science and biomedical research.

Genomic-word-frameworks and the genomic methylation data disclosed herein can be used in a wide variety of applications. For example, such GWF-based model predictions can be used for identifying and treating patients of autism, cancer, and other diseases that benefit from early diagnostics. Said models could also help provide further understanding in discovering causes for (1) phenotypes that are not at present well-understood and (2) multifactorial diseases seemingly caused by both genetic and environmental factors, such as diabetes and alcoholism.

The visual representation of genomic-word-framework analyses can be automatically and intuitively configured so as to quickly convey meaning to those interpreting same. Therefore, at least one embodiment disclosed herein can comprise a distinct aesthetic appearance. Ornamental aspects included in such an embodiment can help further a person's understanding of the potential relationship genomic methylation data has to applications within the physical world (e.g. phenotype).

Methods can be practiced which facilitate use, manufacture, assembly, maintenance, and repair of libraries of DNA methylation motifs which accomplish some or all of the previously stated objectives.

It is a further object, feature, and/or advantage of the present disclosure to provide methods for analyzing methylation signals on stretches of DNA sequences. In some embodiments, the method comprises analyzing a hierarchical cluster on regions of the DNA sequences; grouping a set of selected regions hierarchically into a specified number of clusters; aligning potential DNA sequence motifs from said clusters; and applying digital signal processing to the encoded methylation and physicochemical signals.

The creation and maintenance of libraries can further be incorporated into automated, heuristic analysis processes which constantly refine and improve DNA base interdependence with respect to methylated cytosines until they achieve a base distribution that is statistically nonrandom.

These and/or other objects, features, advantages, aspects, and/or embodiments will become apparent to those skilled in the art after reviewing the following brief and detailed descriptions of the drawings. Furthermore, the present disclosure encompasses aspects and/or embodiments not expressly disclosed but which can be understood from a reading of the present disclosure, including at least: (a) combinations of disclosed aspects and/or embodiments and/or (b) reasonable modifications not shown or described.

### BRIEF DESCRIPTION OF THE DRAWINGS

Several embodiments in which the present disclosure can be practiced are illustrated and described in detail, wherein like reference characters represent like components throughout the several views. The drawings are presented for exemplary purposes and may not be to scale unless otherwise indicated.

FIG. 1 shows a msh1 system composed of four msh1-derived epigenetic states. In Arabidopsis, four distinct plant states originate from MSH1 knockdown or knockout. States 1 and 2 derive directly from msh1 disruption, resulting in highly stress-responsive phenotypes. State 1 at short daylength is variable, including a low-frequency ‘perennial-like’ phenotype16. States 3 and 4 involve interaction of msh1-modified and naïve (wild type) genomes through grafting or crossing, resulting in growth vigor phenotypes.

FIGS. 2A-2H show characteristics of epi-lines derived by crossing msh1 T-DNA mutant with isogenic wild type. 2A shows the phenotypes of different epi-line F3 populations at 34 days after planting (DAP). The lines derive from WT×msh1 crosses, with Epi 24 and Epi 8 from one parental cross, and Epi 10 and Epi 19 from a second parental cross. 2B shows total leaf area (34 DAP), 2C, days to bolting, and 2D, seed weight (mg) are shown for the four populations along with WT control. 2B through 2D show bars representing means±SE. The Mann-Whitney U-test with two-sided alternative hypothesis was used to test significance of the difference of mean between each Epi F3 population and WT control. 2E shows root phenotype of the four different Epi F3 populations grown in sand (33 DAP). 2F shows total leaf area (33 DAP), dry leaf weight (mg), and dry root weight (mg) are shown for the four populations grown on sand along with WT control. 2F-2H show bars represent means±SE. The Mann-Whitney U-test with two-sided alternative hypothesis was used to test significance of the difference of mean between each Epi F3 population and WT control. Significance codes: * p<0.05, ** p<0.01, *** p<0.001, ns—not significant.

FIGS. 3A-3H show tomato plants used for methylome sequencing. 3A shows Rutgers wild type plants at 4 weeks. 3B through 3D show Rutgers msh1 memory plants (State 2, at right in each panel) compared to WT at 4 weeks. 3E through 3G show Rutgers msh1 RNAi transgenic plants (State 1, at right in each panel) compared to WT. 3H shows Epi-line with higher vegetative growth (left 3 plants), denoted as Epi-line High (Epi-H) in subsequent analysis, and epi-line with lower vegetative growth (right 3 plants), denoted as Epi-line Low (Epi-L). Young leaves of 4-week-old plants were collected for whole-genome bisulfite sequencing.

FIGS. 4A-4C show a reversion phenotype in Arabidopsis and soybean. 4A shows plant growth phenotype of three F3 epi-line populations, Epi 10 derived by crossing to msh1 T-DNA mutant, and Epi 6 and Epi 9 derived by crossing to msh1 memory line. Dashed circles indicate putative revertants. Col-0 wild type and msh1 memory are shown as controls. 4B shows field-grown soybean (cv.Thorne) F3 epi-line showing evidence of spontaneous reversion (arrows). Harvest in October followed severe cold, with revertants showing cold tolerance that resembles msh1 memory. 4C shows Principal Component Analysis-Linear Discriminant Analysis of methylome data from three epi-line non-revertant and three revertant full sib F3 progeny compared to three independently-derived msh1 memory plants.

FIG. 5 shows relative DMP frequencies among different Arabidopsis epi-lines. DMPs were assigned to genic, TE-related, and other genomic regions. The centroid of the wild type samples was used as reference. Relative DMP frequencies in each genomic feature were estimated as number of DMPs divided by number of total genomic cytosine positions in each genomic feature. A DMP was defined as ‘hyper’ if cytosine methylation level difference was greater than 0 and defined as ‘hypo’ if methylation level difference was less than 0.

FIGS. 6A-6C show total hyper- and hypo-DMP counts in epi-lines. 6A shows Arabidopsis epi-lines vs WT. 6B shows tomato epi-lines Low (BE) vs High (GE). 6C shows soybean epi-F4 vs epi-F6. Each bar graph represents a single plant in a given population. Cytosine context (CG, CHG, CHH) are shown separately for each plant. A DMP was defined as ‘hyper’ if cytosine methylation level difference was greater than 0 and defined as ‘hypo’ if methylation level difference was less than 0. Control centroid was used a reference.

FIGS. 7A-7C show discrimination of methylation repatterning among different msh1 states. 7A shows hierarchical clustering results with genic methylome data from three different msh states in Arabidopsis: msh1 null mutant (state 1), Col-0/Col-0ₘₛₕ₁ graft progeny (state 3), Epi F3 populations (state 4), and relevant Col-0 controls. 7B shows hierarchical clustering results with individual plant (P) datasets from three different msh1 states in tomato: Rutgers MSH1-RNAi+ (state 1), Rutgers/Rutgers_{msh1-RNAi} graft (HEG) progenies (state 3), and Epi F3 populations (Epi-L and Epi-H) (state 4) with relevant Rutgers controls. 7C shows hierarchical clustering results with TE DMP data from the same Arabidopsis datasets presented in 7A. Individuals were represented as vectors of the sum of Hellinger divergences (HD) at DMP positions within gene regions (7A, 7B) or TE regions (7C). The hierarchical clustering was built using Ward agglomeration method, and Hellinger divergence (HD) was computed by using the centroid of corresponding wild-type samples. HD formula is reported elsewhere.

FIGS. 8A-8E show significant enriched GO pathways by DMG analysis in epi-lines. 8A shows enriched GO pathways shared by Epi 8 and Epi 24 in Arabidopsis. 8B shows enriched GO pathways shared by Epi 10 and Epi 19 in Arabidopsis. 8C shows enriched GO pathways identified by F1 hybrid DMGs from the C24×Ler cross by our analysis. Original methylation data for the F1 hybrid was previously reported. 8D shows enriched GO pathways derived from DMG analysis in Epi-line Low vs Epi-line High comparison in tomato. 8E shows enriched GO pathways based on DMG analysis of the Epi-F4 vs Epi-F6 comparison in soybean. For 8A through 8C, bar graphs represent number of DMGs and dotted line represents −log 10(p-value) for the enrichment test. For 8D through 8E, bar graphs represent number of DMGs and dotted line represents fold enrichment of each enriched pathway. Network Enrichment Analysis Test (NEAT) was used to identify enriched GO pathways. p-values were computed using one-sided hypergeometric distribution test as implemented in NEAT. A complete list of enriched pathways can therefore be provided.

FIGS. 9A-9C show significantly enriched GO pathways based on DEG analysis of epi-lines in Arabidopsis. 9A shows enriched GO pathways from DEGs shared by Epi 8 and Epi 10 leaf tissues. 9B shows enriched GO pathways from DEGs shared by Epi 8 and Epi 24 floral stem tissues and 9C shows root tissues. Bar graph represents DEG numbers and dotted line represents fold enrichment of each enriched pathway. One-sided Fisher's exact test was used to compute FDR as implemented in DAVID GO. A complete list of enriched pathways can therefore be provided.

FIGS. 10A-10B show Protein to Protein Interaction (PPI) network of hubs from subsets of network-related DMGs and DEGs in Arabidopsis Epi 8 and Epi 24 full-sib lines. Epi 8 and Epi 24 represent progeny lines derived from the same cross, with Epi 8 displaying enhanced vegetative growth rate and Epi 24 significantly enhanced in seed yield. The main subnetwork of hubs was obtained with the application of machine learning K-means clustering on the set of 3647 (Epi 8, 10A) and 3523 (Epi 24, 10B) network-related DMGs and DEGs identified in the Arabidopsis epi-line vs WT comparison. The analyses yielded 153 (10A) and 346 (10B) hub genes to form a closely related subnetwork. GO network enrichment analysis from the string application in Cytoscape was used to identify enriched gene function pathways within the network. Size of each node is proportional to its value of node degree and label font size is proportional to its betweenness centrality.

FIGS. 11A-11B show Protein to Protein Interaction (PPI) network of hubs derived from subsets of network-related DMGs and DEGs in soybean epi-lines and tomato graft progeny (HEG) lines. 11A shows a primary subnetwork of hubs obtained with the application of machine learning K-means clustering algorithm on the set of 1264 network-related DMGs and DEGs identified in the soybean Epi-F6 vs Epi-F4 (state 4) comparison. The analysis yielded 421 hub genes forming a closely related subnetwork. GO network enrichment analysis from the string application in Cytoscape was used to identify enriched gene function pathways within the network. 109 genes related to gene expression regulation were selected to present. 11B shows a main subnetwork of hubs obtained with the application of machine learning K-means clustering algorithm on the set of 3173 network-related DMGs and DEGs identified in the tomato Rutgers/Rutgers_{msh1-RNAi} VS Rutgers/Rutgers graft (HEG) progenies (state 3) comparison. Analysis yielded 305 hub genes forming the subnetwork. GO network enrichment analysis from the string application in Cytoscape was used to identify enriched gene function pathways within the network. 126 genes related to gene expression regulation were selected to present. For both 11A and 11B, the size of each node is proportional to its value of node degree and the label font size is proportional to its betweenness centrality. Datasets for soybean DEG and tomato HEG (DMG and DEG) were previously reported.

FIGS. 12A-12D show the relationship of 871 core hub genes to 4 different msh1-derived states and biologically meaningful core networks. 12A shows a Venn diagram of Arabidopsis DMGs identified from four different msh1-derived states (Col-0 genetic background): msh1 mutant (state 1), msh1 memory (state 2), graft progeny (HEG, state 3), and epi-line (Epi 24, state 4). 12B shows an overview of the PPI networks and individual 871 core hub genes. 12C shows hierarchical clustering of individual plant datasets from four different msh1-derived states based on the sum of Bayesian methylation level difference of DMPs over the 871 core genes from 12A. The hierarchical clustering was built using Ward agglomeration method. The Bayesian methylation level difference was computed as described previously. 12D shows main subnetwork of hubs obtained with the application of a machine learning K-means clustering algorithm on the set of 871 core genes from 12A. GO network enrichment analysis from the string application in Cytoscape was used to identify enriched gene function pathways within the network. 67 genes involved in enriched networks were identified. This 67-gene set supplied the RdDM candidate target genes for further study. For both 12B and 12D, the size of each node is proportional to its value of node degree and the label font size is proportional to its betweenness centrality.

FIGS. 13A-13D show investigation of gene methylation repatterning within candidate RdDM target genes that discriminate the four msh1-derived states in Arabidopsis. 13A shows two of the putative cluster motifs identified based on differential gene methylation across four msh1-derived states. Hierarchical clustering on a set of 14-bp regions encompassing DMPs from seven (TOR, SYD, NRPB1, NRPD1, UBP26, SUVR5, UPF1) of the 67 core hub loci followed by DNA multiple sequence alignment of each cluster permitted the identification of methylation motifs. 13B shows a difference of Methylation levels on gene body DMPs within motif cluster 11 in the putative RdDM target gene UBP26. Variations on motif methylation repatterning at DMPs are shown with chromosome and position. Individual detected methylation changes are shown as cross-hatched dots for each plant assayed in each msh1 state, with positive indicating DMP and negative for no detected methylation change. Each line represents a single plant dataset. 13C shows sample DMPs within motif cluster 11 in UBP26 and UPF1 that show dc12/dc13/dc14-sensitivity in graft rootstock (state 1) and graft progeny (state 3) from contrasted mutant rootstock experiments. Note that dc12/dc13/dc14 effects can only be assayed for msh1 and graft progeny data. 13D shows sample putative cluster motifs identified based on differential gene methylation across four msh1-derived states in analyses of the 67 core hub loci followed by DNA multiple sequence alignment of each cluster for methylation motifs.

FIGS. 14A-14D show exemplary cluster motifs encompassing DMPs on gene AT1G50030 (TOR) in Arabidopsis thaliana. 14A shows the cluster motif for cluster 1. 14B shows the cluster motif for cluster 9. 14C shows the cluster motif for cluster 12. 14D shows the cluster motif for cluster 17. Individual consensus nucleotides were evident at variable distance from the target cytosine, which is nucleotide 7 (on the ‘+’ or the ‘−’ strand) within each motif.

FIGS. 15A-15B show power spectral densities of control (15A) and treatment (msh1 mutant, dwarf phenotype) (15B) in A. thaliana. Dots help to visually identify the peaks where spectral differences between the control and the treatment were found.

FIGS. 16A-16B show spectrograms of AT1G50030 (TOR) in A. thaliana. In both 16A and 16B, the top panel is the control and the bottom panel is the treatment (dwarf phenotype). 16A is the spectrogram of the first region and 16B is the spectrogram of the third region. Methylation signal breaks down the power spectrum energy around the periodicity at about the ⅓ frequency (white dash line).

FIGS. 17A-17C show wavelet power spectrums (WPS) of regions AT1G50030.1 (17A), AT1G50030.2 (17B), and AT1G50030.3 (17C) in control (left panel in each) and treatment (dwarf phenotype) (right panel in each) groups. The difference observed between control and treatment groups in the WPS correspond with which base is altered.

FIG. 18 shows a correlogram based on the WPS of 17A. Control versus control is shown in the top panel and control versus treatment (dwarf phenotype) is shown in the bottom panel. In addition to the expected correlation break at region 55-60 bit, the correlation between energy spectrum at regions 30-35 bit and 55-60 bit is lost in the control versus treatment.

FIG. 19 shows the sub-network of 81 DMG-hubs. A PPI network was built on the set of 751 DMGs identified with principal component analysis were analyzed with STRING Cytoscape App. The circles free of cross hatching are highlighted DMGs involved in the biological processes that include nervous system, nervous system development, synapse, neuron projection, ion transport, central nervous system disease, axon guidance, neurogenesis, ion/cation biding, ion/cation transmembrane transport, voltage-gated channel and TRP channels.

FIG. 20 shows the network enrichment analysis identified in the main network of DMG-hub from FIG. 19.

An artisan of ordinary skill in the art need not view, within isolated figure(s), the near infinite number of distinct permutations of features described in the following detailed description to facilitate an understanding of the present disclosure.

### DETAILED DESCRIPTION

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

Genomic-word-framework (GWF) analysis of DNA methylation involves analysis of methylation motifs and digital signal processing. GWFs are stretches of DNA sequence covering differentially methylated positions (DMPs). GWFs are however not to be confused with the concept of a DNA sequence motif. A word-framework (WF) can include one or more motifs. That is, a ‘sentence of WFs’ is also a GWF. DMPs can be identified by methylation analysis with an extension in a statistical programming language, such as the R package described by the inventors of the present application in U.S. Pat. No. 10,913,986. The analysis permits the identification of DNA sequence methylation motifs found in genes with potential epigenetic regulatory functionalities, including those induced by environmental changes or disease.

One potential embodiment of an analytical heuristic described herein has been implemented in an R package named GenomicWordFramework. More particularly, GenomicWordFramework is a utility package to identify the potential genomic word framework (GWF) regions of a hypothetical language. The GWFs facilitate further analysis of DNA sequences that carry genomic signals with the application of digital signal processing (DSP) and machine-learning (ML) tools from other R packages. GenomicWordFramework includes several functions to accomplish reading and data transformation to a suitable form for application of different statistical approaches to data analysis, like clustering algorithms and statistical tests.

GenomicWordFramework can utilize prior identification of DMPs with the R packages specific to methylation analyses, such as those described in U.S. Pat. No. 10,913,986. The use of methylation analyses and GWF analyses described herein can therefore form a pipeline that implements a signal detection and a machine-learning approach permitting filtering of signal from noise at a high rate.

The discriminatory power of GenomicWordFramework was first tested in a small data set of the msh1 mutant system in Arabidopsis thaliana (biological model) and was later applied to a published study of DNA methylation analysis of placental tissues of typically developing and autistic children. Genomic-word-framework analysis is a proven concept compatible with a near limitless number of differentially methylated network-hubs. The differentially methylated network-hubs, previously identified with methylation and network analyses in the autism study, relate to biological processes that include: the nervous system, nervous system development, synapse, neuron projection, central nervous system disease, axon guidance, neurogenesis, ion/cation biding, ion/cation transmembrane transport, voltage-gated channel.

Results indicate that GWF based heuristics can identify DNA sequences of methylation motifs with high order DNA base interdependence with respect to methylated cytosines and a base distribution that is statistically nonrandom. These findings set the basis for further model prediction in patients, not only for autism but also other diseases like cancer that would benefit from early diagnostics.

In other words, GWF analyses are able to identify sets/clusters of synonymous methylation word-frameworks within genes that undergo targeted methylation changes and participate in gene networks that are involved in biological processes relevant to the system under study. GWF further lays the groundwork for the creation of libraries of DNA methylation motifs intended for patient diagnostics and prognostics. GWF analyses therefore substantially increase the utility and value of identification of DMPs and differentially methylated genes (DMGs) with methylation analysis.

### GenomicWordFramework R Package and GWF Identification

GenomicWordFramework is an extension in written in a statical programming language. More particularly, GenomicWordFramework is an R package that is designed for analysis of methylation signals on stretches of DNA sequences that are characterized by not only methylation information, but also physicochemical information around each methylated cytosine (plus adenine in the case of animals and bacteria). The package's functions transform the methylation data to a suitable format to be accessible for further DSP analyses beyond R packages.

GWFs are identified in a methylome in two possible ways: (1) applying the algorithm described in Sanchez et al., “Information Thermodynamics of Cytosine DNA Methylation”, published Mar. 10, 2016, which is herein incorporated by reference in its entirety; and (2) direct extraction of DNA sequence stretches covering specified numbers of DNA bases upstream and downstream of DMPs. The GWFs obtained with the first approach can be further analyzed with digital signal processing (DSP) tools. GWFs obtained with the second approach can be clustered into groups of aligned motifs and further tested to evaluate departure of each of multiple sequence alignments (MSA) from random Monte Carlo simulated MSAs. Statistically significant motif regions are extended for further applications of DSP analyses.

### Binary Coding of Methylation Signal

All information needed to search for a DNA motif, DNA sequence and modified nucleotide base can be stored in a singular binary string using a triplet representation of each base taking into account the number of hydrogen bonds in the Watson-Crick base pair, the chemical type of the DNA base (pyrimidine and purine), and the base modification status. By convention, the DNA sequence is referred to the positive strand. For example, the following encoding of DNA bases can be used to study the methylation signal:
Methylation status: 1 (methylated) and 0 (unmethylated)
Strong-Weak base-pair interaction: 1 (three hydrogen bonds, S class) and 0 (two hydrogen bonds, W class)
Chemical type: 1 (pyrimidine, Y class) and 0 (purine, R class).
Radical content: 1 (base containing amino (NH_3, M class)) and 0 (base containing keto (C═O) group (K class))

Each base will comprise a binary string:
the signal mC, methylated pyrimidine with three hydrogen bonds, on the positive strand: 111.
the signal mC, methylated pyrimidine with three hydrogen bonds, on negative strand (base G on the ‘+’ positive strand): 110.
C, pyrimidine with three hydrogen bonds, M class: 011
G, purine with three hydrogen bonds, K class: 010
T, pyrimidine with two hydrogen bonds, K class: 001
the signal mA, methylated purine with two hydrogen bonds, on the positive strand: 100.
the signal mA, methylated purine with two hydrogen bonds, on the negative strand (base T on the ‘+’ positive strand): 101
A, purine with two hydrogen bonds, M class: 000
U, same as for ‘T’: 001.

Each base will comprise a complex number (a+ib), the cyclic group integrated by the 8^{th} roots of unity: e

$$\frac{2 π k i}{8} ,$$

where k=0, . . . , 7.
the signal mC, methylated pyrimidine with three hydrogen bonds, on the positive strand: 1.
the signal mC, methylated pyrimidine with three hydrogen bonds, on negative strand (base G on the ‘+’ positive strand): −1.
C, pyrimidine with three hydrogen bonds, S class:

$$\frac{1 + i}{\sqrt{2}}$$

G, purine with three hydrogen bonds, S class:

$$\frac{1 - i}{\sqrt{2}}$$

T, pyrimidine with two hydrogen bonds, W class:

$$\frac{- 1 + i}{\sqrt{2}}$$

the signal mA, methylated purine with two hydrogen bonds, on the positive strand: i.
the signal mA, methylated purine with two hydrogen bonds, on the negative strand (base T on the ‘+’ positive strand): −i
A, purine with two hydrogen bonds, W class:

$$\frac{- 1 - i}{\sqrt{2}}$$

U, same as for ‘T’:

$$\frac{- 1 + i}{\sqrt{2}} .$$

More than one complex encoding is possible. Encoding based on a group structure (here an Abelian group) can be preferred for DSP analysis. This is a group structure defined on the set of DNA bases including methylated cytosine and adenine member of the alphabet, as described further below. Analysis of a complex signal in GWF R package can be difficult, complex encoding(s) and signal(s) can later be exported to other languages like C++, Python, or MatLab.

Specific details for the implementation of this encoding are provided in the documentation of a function that encodes a previous detected binary signal of 0s and 1s from a DNA sequence into a numerical code defined by the user. Given two objects, one carrying the signal and the other one carrying the DNA sequence, such a function can perform the encoding set out by the user. The binary encoding of the methylation signals permits the incorporation of the physicochemical information in the DSP analyses of DNA sequence motifs.

### Motif Clustering

Small regions of usually 7-30 bp spanning DMPs on at least three samples were identified and considered as DNA methylation motif candidates in 67 genes. A distance matrix can be estimated on the set of selected regions using a function from the R package that computes the matrix distance between the aligned sequences from each multiple sequence alignment (MSA). Next, a hierarchical cluster analysis on the set of selected regions (using the previously estimated distance matrix) can be accomplished with a function that utilizes a matrix of a selected Information Divergence to group the selected regions into 100 clusters. It is to be appreciated Hellinger divergence is only one of the possible information divergences that can be estimated and applied here. For example, J-divergence is more appropriated for application intended to extract new knowledge in terms of information-thermodynamics of the epigenome phenomena. J-divergence is the symmetric version of relative entropy.

An unweighted pair group method with arithmetic mean (“UPGMA”) approach was applied as agglomeration algorithm. In one embodiment, clusters with less than ten (10) regions on it are discarded. DNA multiple sequence alignment on each cluster of sequences can be accomplished with the MUltiple Sequence Comparison by Log-Expectation (MUSCLE) algorithm implemented on an R package for the analysis and comprehension of genomic data generated by wet lab experiments in molecular biology.

A further portioning of the set of motifs can be applied for downstream analysis. There is a wide spectrum of clustering algorithms that can be applied. For example, fast k-medoids clustering can be applied and implemented using algorithms of distance-based k-medoids clustering: such as simple and fast k-medoids, ranked k-medoids, and increasing number of clusters in k-medoids. In one embodiment, said algorithms are those included in the kmed R package.

The cluster results can be plotted in a marked barplot or pca biplot. The final partition into clusters depends on the clustering algorithm applied and their corresponding parameter settings, including the type of metric applied to compute the distance matrix required for clustering algorithm. Methylation motifs are objective DNA sequence features, and the applied clustering algorithm is only a supporting tool that leads to motif identification.

### Motif Score

The motif score sⱼₖ of the aligned sequences j and k can be defined in an intuitive way: as the logarithm base 2 of the number of matched bases found in the alignment. Formally:

$${s}_{j k} = {log}_{2} {\sum }_{i = 1}^{N} {I}_{j k}^{i}$$

where

$${I}_{jk} = \{ \begin{matrix}1 if {b}_{j}^{i} = {b}_{k}^{i} \\ 0 otherwise\end{matrix}$$

for every base position i on sequences j and k. Then, the maximum motifs score is: Max{sⱼₖ}=log₂N. Next, the motifs score in a MSA is defined as:

$$S = \frac{1}{m} {\sum }_{j = 1}^{M - 1} {\sum }_{k = j + 1}^{M} {s}_{j k}$$

For a MSA with M sequences of length N each, the number of pairwise comparisons is:

$$m = \frac{M \times ( M - 1 )}{2} .$$

As a result, for a fixed value of the motif size, the perfect MSA of DNA sequence motifs will have the maximum score: Max{S}=log₂N. In other words, in this modeling, the maximum amount of information carried by an MSA is: Iₘₐₓ=log₂N, and the amount of information (the uncertainty change) carried by a MSA is given by the expression:

$$I = - ( S - {log}_{2} N )$$

The same result is obtained if the letter frequencies in the MSA and Shannon entropies, before (perfect alignment) and after, are estimated instead of the matches. Then, alignment information is computed as:

$$I = - ( {H}_{after} - {H}_{before} )$$

### Monte Carlos Testing of MSA Randomness

A Monte Carlos testing (MCT) on how a given DNA multiple sequence alignment differs from randomly generated MSAs was implemented in an R function included in the GenomicWordFramework R package. To accomplish the MCT, it is assumed:
The count vectors nₖ summarizing the observed DNA base frequencies in N column (k=A, C, G, T) will be distributed according to the multinomial distribution with parameters pk.
In Bayesian framework, since Dirichlet distribution is the conjugate prior distribution of the multinomial distribution, it is assumed one of the two approaches to generate the parameter vector:
   If the number of genes or genomic regions from where the motifs are identified is small: the parameter vector P=(pA, pC, pG, pT) is drawn from a Dirichlet distribution D_αP with parameter vector α=(α1, α2, α3, α4).
   If the number of genes or genomic regions from where the motifs are identified is large: the parameter vector P=(pA, pC, pG, pT) is drawn from a Dirichlet mixture distribution, where each Dirichlet component D_(α_i Q_i) has parameters α_i=(α_i1,α_i2,α_i3,α_i4) and Q=(qA, qC, qG, qT). Formally:

$$P ( P ) = {\sum }_{i} {π}_{i} {𝒟}_{{α}_{i} {Q}_{i}}$$

Where Σᵢπᵢ=1.
DNA bases are generated independently according to the distribution pk.

### Dirichlet Distribution Estimation

The parameter vector α=(α₁, . . . , αₖ) for a specific Dirichlet distribution is estimated from the whole set of identified DNA motif candidates. Given a matrix of DNA methylation motifs with N columns corresponding to the whole set of identified motif candidates, the frequency of each DNA base in each column is tallied, resulting in a four-dimensional vector of counts for each column in the data set, where each vector coordinate carries the absolute frequency of one of the four DNA bases in a given alignment column. The resulting N×4 matrix of counts is the raw count data used in the parameter estimation of Dirichlet distribution applying a function from the R package that estimates a family of continuous multivariate probability distributions: a multivariate generalization of the Beta distribution. Next, random DNA MSA sequences are generated according to the estimated Dirichlet distribution with a probability density function (PDF) or cumulative density function (CDF) from the R package. That is, random DNA MSA were generated sampling from the estimated Dirichlet distribution.

Monte Carlo p-Value

For MSAs of fixed length N, the log₂ N is a constant, so for the purposes of MCT it is sufficient to consider the score statistic given in Eq. 2 and to evaluate how much an observed aligned motif differs statistically from Monte Carlo simulated aligned sequences. The Monte Carlo p-value is estimated as:

$${p}_{value} = \frac{1}{{N}_{S} + 1} {\sum }_{i = 1}^{{N}_{s}} {δ}_{i} ( {S}_{i} , {S}_{0} )$$

Where S₀ stands for the alignment score of the MSA to be tested, Sᵢ is the alignment score for the i^{th} Monte Carlo simulated MSA, and

$${δ}_{i} ( {S}_{i} , {S}_{0} ) = \{ \begin{matrix}1 if {S}_{i} \geq {S}_{0} \\ 0 otherwise\end{matrix} .$$

It is important to notice that the raw observed frequencies from a small matrix of motifs are often poor approximations to the distribution of DNA bases among all motifs that the model is supposed to represent. However, for a typical analysis of 50 or more genes, the matrix of motifs needed for the Dirichlet distribution model estimation would, in general, carry thousands of motifs.

### Digital Signal Processing of the Binary-Encoded Methylation Signal

The binary-encoded methylation signal is raw data for digital signal processing (DSP) tools. There is a huge number of possible applications of DSP tools. The GenomicWordFramework R package can include some the application of wavelet spectrogram via wavelet transform, as well as the traditional Fourier power spectrum and spectrogram.

The Multiplicative Group of DNA Extended Alphabet with Methylated Bases

Let [symbol] ={C^{m}, C, A^{m}, T, C₋^{m}, A, A₋^{m}, G} be the ordered set of DNA bases plus the methylated adenine (A^{m}) and cytosine (C^{m}) in the positive strand and in the negative strand A₋^{m} and C^{m}. Let us define on [symbol] a multiplicative group ( [symbol] ,x) with multiplication operation ‘x’, where C₋^{m} is the multiplication unit and the unmethylated DNA complementary bases are algebraic complementary as well, i.e.: C×G=C^{m} and A×T=C^{m}. The last algebraic-biophysical constraints are hold if base C is the group generator, i.e., the generator condition C⁸=C^{m} and the condition C×G=C^{m} imply C⁷=G. Since C³×C⁵=C⁸, setting C³=T implies C⁵=A. Preserving the order of bases in the set, we have C×C=C²=A^{m}, C⁴=C₋^{m}, C⁶=A₋^{m}.

The group ( [symbol] ,x) defined above is an Abelian cyclic group isomorphic to the cyclic group integrated by the 8^{th} roots of unity:

$${e}^{\frac{2 π ki}{8}} ,$$

where k=0, . . . , 7. Although we can accomplish the symbolic algebraic operations on ( [symbol] ,x), for the sake of concrete applications in computational biology and in bioinformatics, it is convenient to operate with the cyclic group defined on the set

$$\{ {e}^{\frac{2 π ki}{8}} \vert k = 0 , … , 7 \} .$$

The elements of this group, written in the order sets by the bijective mapping

$$↔ \{ {e}^{\frac{2 π ki}{8}} \vert k = 0 , … , 7 \} ,$$

$$are : \{ 1 , \frac{1 + i}{\sqrt{2}} , i , - \frac{1 - i}{\sqrt{2}} , - 1 , - \frac{1 + i}{\sqrt{2}} , - i , \frac{1 - i}{\sqrt{2}} \} ,$$

where i is the imaginary unit defined in the set of complex numbers.
Complex Encoding of Methylation Signal with GWF R Package
library(GenomicWordFramework)

We will use methylation signal from three gene regions from Arabidopsis thaliana data(at_signal, at_gene_seq, package=“GenomicWordFramework”)

Each base will comprise a complex number (a+ib), the cyclic group integrated by the 8th roots of unity

$${e}^{\frac{2 π ki}{8}} ,$$

where k=0, . . . , 7.
the signal mC, methylated pyrimidine with three hydrogen bonds, on the positive strand: 1.
the signal mC, methylated pyrimidine with three hydrogen bonds, on negative strand (base G on the ‘+’ positive strand): −1.
C, pyrimidine with three hydrogen bonds, S class:

$$\frac{1 + i}{\sqrt{2}}$$

G, purine with three hydrogen bonds, S class:

$$\frac{1 - i}{\sqrt{2}}$$

T, pyrimidine with two hydrogen bonds, W class:

$$\frac{- 1 + i}{\sqrt{2}}$$

the signal mA, methylated purine with two hydrogen bonds, on the positive strand: i.
the signal mA, methylated purine with two hydrogen bonds, on the negative strand (base T on the ‘+’ positive strand): −i
A, purine with two hydrogen bonds, W class:

$$\frac{- 1 - i}{\sqrt{2}}$$

U, same as for ‘T’:

$$\frac{- 1 + i}{\sqrt{2}} .$$

In GWF R package, the encoding of the methylation signal is accomplished with function signalEncoding. In the current case, we used the generator of the group is:

$$W = \frac{1 + i}{\sqrt{2}}$$

to easily set the encoding:

w = (1 + 1i)/sqrt(2)
enc_signal <- signalEncoding(

```
          signal = at_signal,
          GR = at_gene_seq,
          S = “1”,
          Sc = “−1”,
          Cs = paste0(w),
          Gs = paste0(w\hat{ }5),
          Ts = paste0(w\hat{ }3),
          As = paste0(w\hat{ }5),
          Us = paste0(w\hat{ }5),
          Ns= paste0(w\hat{ }7),
          OS = “1i”,
          OC = “−1i”,
          Bs = “C”,
          O = “A”,
          verbose = FALSE)
```

enc_signal

| | |
|---|---|
| ## | GroupedSignalEncList object of length: 10 |
| ## | names(10): col1 col2 col3 col_1 col_2 dw 1 dw 2 dw 3 dw_1 dw_2 |
| ## | ------- |
| ## | $col1 |
| ## | SignalEncList object of length: 7 |
| ## | names(7): AT1G50030 AT1G63020 AT2G23740 AT2G28290 AT3G49600 AT4G35800 |

AT5G47010

| | |
|---|---|
| ## | ------- |
| ## | $AT1G50030 |
| ## | SignalEnc object with 17748 ranges and 3 metadata columns: |
| ## | seqnames ranges strand \| signal seq |
| ## | <Rle> <IRanges> <Rle> \| <numerica> <character> |
| ## | [1] 1 18522248 − \| 0 A |
| ## | [2] 1 18522249 − \| 0 A |
| ## | [3] 1 18522250 − \| 0 C |
| ## | [4] 1 18522251 − \| 0 A |
| ## | [5] 1 18522252 − \| 0 A |
| ## | ... ... ... ... . ... ... |
| ## | [17744] 1 18539991 − \| 0 T |
| ## | [17745] 1 18539992 − \| 0 T |
| ## | [17746] 1 18539993 − \| 0 T |
| ## | [17747] 1 18539994 − \| 0 T |
| ## | [17748] 1 18539995 − \| 0 T |
| ## | encoding |
| ## | <complex> |
| ## | [1] −0.707106781186547−0.. |
| ## | [2] −0.707106781186547−0.. |
| ## | [3] 0.707106781186547+0... |
| ## | [4] −0.707106781186547−0.. |
| ## | [5] −0.707106781186547−0.. |
| ## | ... ... |
| ## | [17744] −0.707106781186547+0.. |
| ## | [17745] −0.707106781186547+0.. |
| ## | [17746] −0.707106781186547+0.. |
| ## | [17747] −0.707106781186547+0.. |
| ## | [17748] −0.707106781186547+0.. |
| ## | ------- |
| ## | seqinfo: 1 sequence from an unspecified genome; no seqlengths |
| ## | |
| ## | ... |
| ## | <6 more SignalEnc elements> |
| ## | ------- |
| ## | |
| ## | ... |
| ## | <9 more SignalEncList elements> |
| ## | ------- |

The signal ready for exporting can be retrieved with function getEncoding:

enc <- getEncoding(enc_signal$col1)
enc
## encCompSignalList object of length: 7
## names(7): AT1G50030 AT1G63020 AT2G23740 AT2G28290 AT3G49600 AT4G35800 AT5G
47010
## -------
## $AT1G50030
## encCompSignal object of length: 17748
## [1] −0.707106781186547-0.707106781186547i ...
## Element genomic coordinates in the slot object@SeqRanges
## Element encoding coordinates in the slot object@EncRanges
## -------
##
## ...
## <6 more encCompSignal elements>
## -------

The whole matrix of encoded signal can be retrieved:

enc <- getSignalMatrix(enc_signal, num.cores = 60)
enc

| | |
|---|---|
| ## | SignalMatrixList object of length: 10 |
| ## | names(10): col1 col2 col3 col_1 col_2 dw 1 dw 2 dw 3 dw_1 dw_2 |
| ## | ------- |
| ## | $col1 |
| ## | compSignalMatrix with 7 ranges and 17748 metadata columns: |
| ## | seqnames ranges strand \| V1 |
| ## | <Rle> <IRanges> <Rle> \| <complex> |
| ## | [1] 1 18522248-18539995 + \| −0.707106781186547−0.. |
| ## | [2] 1 23354685-23362589 + \| −0.707106781186547+0.. |
| ## | [3] 2 10097265-10103398 + \| −0.707106781186547+0.. |
| ## | [4] 2 12056213-12073279 + \| 0.707106781186547+0... |
| ## | [5] 3 18380417-18387239 + \| −0.707106781186547+0.. |
| ## | [6] 4 16960558-16968128 + \| 0.707106781186547+0... |
| ## | [7] 5 19071824-19079453 + \| 0.707106781186547+0... |
| ## | V2 V3 ... V17746 |
| ## | <complex> <complex> ... <complex> |
| ## | [1] −0.707106781186547−0.. 0.707106781186547+0... ... −0.707106781186547+0.. |
| ## | [2] 0.707106781186547+0... −0.707106781186547−0.. ... <NA> |
| ## | [3] −0.707106781186547+0.. 0.707106781186547+0... ... <NA> |
| ## | [4] −0.707106781186547+0.. −0.707106781186547+0.. ... <NA> |
| ## | [5] −0.707106781186547+0.. −0.707106781186547+0.. ... <NA> |
| ## | [6] 0.707106781186547+0... 0.707106781186547+0... ... <NA> |
| ## | [7] −0.707106781186547+0.. 0.707106781186547+0... ... <NA> |
| ## | V17747 V17748 |
| ## | <complex> <complex> |
| ## | [1] −0.707106781186547+0.. −0.707106781186547+0.. |
| ## | [2] <NA> <NA> |
| ## | [3] <NA> <NA> |
| ## | [4] <NA> <NA> |
| ## | [5] <NA> <NA> |
| ## | [6] <NA> <NA> |
| ## | [7] <NA> <NA> |
| ## | ------- |
| ## | seqinfo: 5 sequences from an unspecified genome; no seqlengths |
| ## | |
| ## | ... |
| ## | <9 more compSignalMatrix elements> |
| ## | ------- |

The matrix carrying the signal can be exported to Python or MatLab.

enc_signal_matrix <- getSigBySeqname(enc$col1, seqname = “1”)
enc_signal_matrix

| | |
|---|---|
| ## | |
| ## | LargeMatrix object with 2 rows and 17748 columns: |
| ## | ------- |
| ## | V1 V2 V3 |
| ## | 1.1 −0.7071068−0.7071068i −0.7071068−0.7071068i 0.7071068+0.7071068i |
| ## | 1.2 −0.7071068+0.7071068i 0.7071068+0.7071068i −0.7071068−0.7071068i |
| ## | V4 V5... V17744 |
| ## | 1.1 −0.7071068−0.7071068i −0.7071068−0.7071068i ... −0.7071068+0.7071068i |
| ## | 1.2 −0.7071068−0.7071068i −0.7071068−0.7071068i ... NA |
| ## | V17745 V17746 V17747 |
| ## | 1.1 −0.7071068+0.7071068i −0.7071068+0.7071068i −0.7071068+0.7071068i |
| ## | 1.2 NA NA NA |
| ## | V17748 |
| ## | 1.1 −0.7071068+0.7071068i |
| ## | 1.2 NA |
| ## | ------- |

The whole exportable numerical matrix (showing two rows and the only the 20 first columns):

as.matrix(enc_signal_matrix)[1:2, 1:20]
## V1 V2 V3
## 1 −0.7071068−0.7071068i −0.7071068−0.7071068i 0.7071068−0.7071068i
## 1 −0.7071068+0.7071068i 0.7071068+0.7071068i −0.7071068−0.7071068i
## V4 V5 V6
## 1 −0.7071068−0.7071068i −0.7071068−0.7071068i −0.7071068−0.7071068i
## 1 −0.7071068−0.7071068i −0.7071068−0.7071068i −0.7071068−0.7071068i
## V7 V8 V9
## 1 −0.7071068−0.7071068i −0.7071068+0.7071068i −0.7071068+0.7071068i
## 1 −0.7071068−0.7071068i −0.7071068+0.7071068i −0.7071068−0.7071068i
## V10 V11 V12
## 1 −0.7071068−0.7071068i −0.7071068+0.7071068i −0.7071068−0.7071068i
## 1 −0.7071068+0.7071068i −0.7071068+0.7071068i −0.7071068+0.7071068i
## V13 V14 V15
## 1 −0.7071068+0.7071068i −0.7071068+0.7071068i −0.7071068−0.7071068i
## 1 0.7071068+0.7071068i 0.7071068−0.7071068i −0.7071068+0.7071068i
## V16 V17 V18
## 1 −0.7071068−0.7071068i −0.7071068+0.7071068i −0.7071068+0.7071068i
## 1 −0.7071068−0.7071068i −0.7071068+0.7071068i −0.7071068−0.7071068i
## V19 V20
## 1 −0.7071068−0.7071068i −0.7071068+0.7071068i
## 1 −0.7071068+0.7071068i −0.7071068+0.7071068i

### Embodiments

The following non-limiting numbered embodiments also form part of the present disclosure:
1. An extension for a general purpose programming language or a statistical programming language, said extension comprising:
   algorithm(s) that analyzes methylation signals on stretches of DNA sequences, said DNA sequences being characterized by:
i. methylation information; and
ii physicochemical information around each methylated cytosine;
   wherein the algorithm(s) include one or more functions that can:
estimate a distance matrix on a set of selected regions of said DNA sequences;
analyze a hierarchical cluster on the set of selected regions;
group the set of selected regions into a specified number of clusters; and
align multiple DNA sequences from the clusters into methylation motifs.
2. The extension of embodiment 1, wherein the extension is written in the R statistical language.
3. The extension of any one of embodiments 1-2, further comprising a digital signal processing (DSP) tool available in another programing language.
4. The extension of embodiment 3, wherein the another programming language is C++, Python, or MatLab.
5. The extension of any one of embodiments 1-4, further comprising:
encoding the DNA sequence and physicochemical properties of DNA base into a numerical or complex signal further comprising the DSP.
6. The extension of embodiment 5, wherein said encoding is based on a group structure.
7. The extension of embodiment 6, wherein said group structure is an Abelian group.
8. The extension of any one of embodiments 1-7, wherein the set of selected regions is grouped into groups of at least 100 clusters.
9. The extension of any one of embodiments 1-8, wherein clusters with less than ten regions are discarded.
10. The extension of any one of embodiments 1-9, wherein the multiple DNA sequences are aligned using a multiple sequence comparison by a log-expectation algorithm.
11. The extension of embodiment 5, further comprising: exporting the numerical or complex signal into C++, Python, or MatLab.
12. The extension of any one of embodiments 1-12, wherein the methylation motifs are further grouped for downstream analysis.
13. The extension of embodiment 12, wherein the methylation motifs are further grouped using a clustering algorithm.
14. The extension of embodiment 13, wherein the clustering algorithm is a distance-based k-medoids clustering algorithm.
15. The extension of any one of embodiments 1-14, further comprising:
encoding the methylation and physicochemical signals of DNA bases into one binary, numerical, or complex signal.
16. The extension of embodiment 15, wherein said encoding is based on a group structure.
17. The extension of embodiment 16, wherein said group structure is an Abelian group.
18. The extension of any one of embodiments 15-17, further comprising: conducting a power spectral analysis on the encoded methylation and physicochemical signals.
19. The extension of embodiment 18, wherein the power spectral analysis is a wavelet power spectral analysis (WPS).
20. The extension of embodiment 19, further comprising: computing a correlogram based on the WPS.
21. A computerized heuristic comprising:
a high order DNA base interdependence with respect to methylated cytosines; and
a base distribution that is statistically nonrandom.
22. The computerized heuristic of embodiment 21, wherein said high order DNA interdependence and said base distribution result from analysis of at least one hierarchical cluster on a region of a DNA sequence and aligning multiple DNA sequences from the clusters into methylation motifs.
23. A method for analyzing methylation signals on stretches of DNA sequences, said method comprising:
analyzing a hierarchical cluster on regions of the DNA sequences;
grouping a set of selected regions hierarchically into a specified number of clusters; aligning potential DNA sequence motifs from said clusters; and
applying digital signal processing to the encoded methylation and physicochemical signals.
24. The method of embodiment 23, wherein the set of selected regions is grouped into groups of at least 100 clusters.
25. The method of any one of embodiments 23-24, wherein clusters with less than ten regions are discarded.
26. The method of any one of embodiments 23-25, wherein said encoded methylation and physicochemical signals are encoded based on group structure.
27. The method of embodiment 26, wherein said group structure is an Abelian group.
28. The method of any one of embodiments 23-27, wherein the potential DNA sequences are aligned using a multiple sequence comparison by log-expectation algorithm.
29. The method of any one of embodiments 23-28, further comprising:
conducting a power spectral analysis on the encoded methylation and physicochemical signals.
30. The method of embodiment 29, wherein the DSP is applied through use of a genomic-word-framework (GWF) R package.
31. The method of embodiment 30, wherein the GWF R package includes one or more clustering algorithms.
32. The method of any one of embodiments 30-31, further comprising exporting the GWF R package to be analyzed with other DSP tools in a different programming language.
33. The method of embodiment 32, wherein the different programming language is C++, Python, or MatLab.
34. The method of any one of embodiments 23-33, further comprising:
using methylation analyses to identify differentially methylated positions (DMPs) that form part of the methylation signals.
35. The method of any one of embodiments 23-34, further comprising:
evaluating departure of each of multiple sequence alignments (MSA) from random Monte Carlo simulated MSAs.
36. The method of any one of embodiments 23-35, further comprising:
analyzing genes with epigenetic regulatory functionalities to diagnose or cure a disease.
37. The method of embodiment 36, wherein the disease is autism or cancer.
38. A computerized heuristic for analyzing genetic data comprising:
(1) a statistic of an information divergence (ID) estimated for each gene carrying at least one differentially methylated position (DMP) on the gene or on a promoter region;
(2) principal component analysis (PCA), wherein the first k-th components carrying 1% or more of the whole sample variance are considered in the downstream analysis;
(3) computation of a correlation matrix carrying the pairwise gene correlation, represented as vectors of PCs;
(4) analysis of the correlation matrix for a network; and
(5) contribution of each gene to the discrimination of phenotypes evaluated in terms of the fraction of a cumulative variance from a whole sample variance carried by the gene.
39. The computerized heuristic of embodiment 38, wherein the ID is selected from the group consisting of: Hellinger divergence/distance, J divergence, and total variation distance.
40. The computerized heuristic of embodiment 39, wherein the ID is J divergence.
41. The computerized heuristic of any one of embodiments 38-40, wherein the PCA is applied with a function such that genes are represented as k-dimensional vectors of PCs, and further wherein the square of each coordinate carries the vector contribution in terms of variance to the treatment discrimination from the control group.
42. The computerized heuristic of any one of embodiments 38-41, wherein the PCA is applied with a pcaLDA function.
43. The computerized heuristic of any one of embodiments 38-42, wherein the correlation matrix comprises a weighted correlation network (WCN), wherein the WCN and the network are analyzed, and the network is a Protein to Protein Interaction (PPI) network.
44. The computerized heuristic of embodiment 43, further comprising a comparison of results from the WCN and the PPI network to identified consistent relationships and epigenetic gene contributions to the phenotypes.
45. The computerized heuristic of any one of embodiments 38-44, further comprising a magnitude computed as the Euclidean Norm of the gene represented as a vector of k PCs.
46. The computerized heuristic of any one of embodiments 38-45, wherein the network relates to one or more biological processes.
47. A method of reducing computation load, said method comprising:
identifying differentially methylated genes (DMGs) on stretches of DNA sequences using methylation analysis;
integrating said DMGs to gene networks; and
identifying network hubs.
48. The method of embodiment 47, wherein the network hubs are identified via Protein to Protein Interaction (PPI) network analysis and weighted correlation network (WCN) analysis.
49. The method of embodiment 48, further comprising comparing results from the WCN and the PPI network to identified consistent relationships and epigenetic gene contributions to the phenotypes.
50. The method of any one of embodiments 47-49, wherein the network hubs relate to one or more biological processes.

### Example 1: Arabidopsis Datasets

For concrete application on raw genomic data, the concepts, algorithms, and formulas must be implemented in some computation language. While it is to be appreciated a wide variety of computation languages could be employed, this example chooses the R statistical language and relies on the R package named GenomicWordFramework. The steps and results obtained in this example come from the application of the heuristic to a concrete (and small) experimental data set. Results for a larger data set (in humans) are presented in a later example.

library(GenomicWordFramework)
library(MethylIT.utils)
library(ggplot2)
library(ggseqlogo)
library(rtracklayer)

The package goal is to derive objects that can be useful for further applications of DSP and ML tools available in others R packages. However, the application of some basic DSP tools is provided as well.

Signal Analysis of an Arabidopsis thaliana Experimental Dataset

An example with empirical methylation signal data is illustrated using a dataset included with the package. The experimental dataset carries the methylation levels from Arabidopsis Columbia-0 ecotype (Col-0) and the msh1 mutant (dwarf phenotype). The methylation data, derived with the previous application of methylation analyses, are included as dataset with package. The DMP data set can be loaded from the package:

data(at_meth, package = “GenomicWordFramework”)
as(at_meth, “GRangesList”)

| | |
|---|---|
| #> | GRangesList object of length 10: |
| #> | $col1 |
| #> | GRanges object with 30682 ranges and 11 metadata columns: |
| #> | seqnames ranges strand \| c1 t1 c2 t2 |
| #> | <Rle> <IRanges <Rle> \| <numeric> <numeric> <numeric> <numeric> |
| #> | [1] 1 24196 + \| 18 30 4 0 |
| #> | [2] 1 24331 + \| 36 21 0 7 |
| #> | [3] 1 29033 − \| 19 21 7 0 |
| #> | [4] 1 30699 + \| 34 21 0 7 |
| #> | [5] 1 30753 + \| 13 33 6 0 |
| #> | ... ... ... ... . ... ... ... ... |
| #> | [30678] 5 26289565 − \| 26 15 1 4 |
| #> | [30679] 5 26289578 − \| 22 18 0 4 |
| #> | [30680] 5 26464861 − \| 25 8 1 2 |
| #> | [30681] 5 26835237 − \| 24 11 1 3 |
| #> | [30682] 5 26891687 − \| 24 27 0 5 |
| #> | p1 p2 TV bay.TV hdiv jdiv |
| #> | <numeric> <numeric> <numeric> <numeric> <numeric> <numeric> |
| #> | [1] 0.445992 0.816332 0.625000 0.370340 1.40872 0.456433 |
| #> | [2] 0.656767 0.317068 −0.631579 −0.339699 1.67359 0.347027 |
| #> | [3] 0.538544 0.858989 0.525000 0.320445 1.73403 0.381965 |
| #> | [4] 0.646100 0.317068 −0.618182 −0.329032 1.56153 0.324980 |
| #> | [5] 0.371781 0.847156 0.717391 0.475376 3.14184 0.767111 |
| #> | ... ... ... ... ... ... ... |
| #> | [30678] 0.551769 0.247140 −0.434146 −0.304629 1.046129 0.290444 |
| #> | [30679] 0.483547 0.187724 −0.550000 −0.295823 0.909553 0.298541 |
| #> | [30680] 0.630175 0.289492 −0.424242 −0.340683 0.862905 0.351626 |
| #> | [30681] 0.580545 0.266645 −0.435714 −0.313901 0.911927 0.302677 |
| #> | [30682] 0.429618 0.173993 −0.470588 −0.255626 0.864966 0.234953 |
| #> | wprob |
| #> | <numeric> |
| #> | [1] 5.75270e−04 |
| #> | [2] 5.25963e−05 |
| #> | [3] 2.89143e−05 |
| #> | [4] 1.51431e−04 |
| #> | [5] 7.62181e−14 |
| #> | ... ... |
| #> | [30678] 0.00148034 |
| #> | [30679] 0.00299411 |
| #> | [30680] 0.00381814 |
| #> | [30681] 0.00295741 |
| #> | [30682] 0.00377722 |
| #> | ------- |
| #> | seqinfo: 5 sequences from an unspecified genome; no seqlengths |
| #> | |
| #> | ... |
| #> | <9 more elements> |

To Retrieve a Genomic Signal from its DNA Sequence

Here, the coordinates of Arabidopsis annotated genes given in the GTF file downloaded from ftp://ftp.ensemblgenomes.org/pub/plants/release-49/gtf/arabidopsis_thaliana/.

data(“at_genes”)
at_genes <- at_genes[ match(c(“TOR”, “SYD”, “NRPB1”, “NRPD1”, “UBP26”, “SUVR5”, “UP
F1”),
at_genes$gene_name) ]
at_genes

| | |
|---|---|
| #> | GRanges object with 7 ranges and 2 metadata columns: |
| #> | seqnames ranges strand \| gene_id gene_name |
| #> | <Rle> <IRanges> <Rle> \| <character> <character> |
| #> | [1] 1 18522248-18539995 − \| AT1G50030 TOR |
| #> | [2] 2 12056213-12073279 + \| AT2G28290 SYD |
| #> | [3] 4 16960558-16968128 − \| AT4G35800 NRPB1 |
| #> | [4] 1 23354685-23362589 − \| AT1G63020 NRPD1 |
| #> | [5] 3 18380417-18387239 − \| AT3G49600 UBP26 |
| #> | [6] 2 10097265-10103398 + \| AT2G23740 SUVR5 |
| #> | [7] 5 19071824-19079453 + \| AT5G47010 UPF1 |
| #> | ------- |
| #> | seqinfo: 5 sequences from an unspecified genome; no seqlengths |

The coordinates of genes are used to get the corresponding DNA sequences carrying the signal. For the sake of brevity, the DNA sequence is available with the package.

```
url <- paste0(“ftp://ftp.ensemblgenomes.org/pub/plants/release-49/”,
  “fasta/arabidopsis_thaliana/dna/Arabidopsis_thaliana.”,
  “TAIR10.dna.chromosome.”, 1:5, “.fa.gz”)
at_gene_seq = DNAset2GRSeqset(filepath = url, coordinates = at_genes,
    col_id = 1L, chrs = paste0(1:5),
    num.cores = 4L)
```

The sequences for this example are provided with the package.

data(at_gene_seq)
at_gene_seq

| | |
|---|---|
| #> | GRSegsetList object of length: 7 |
| #> | names(7): AT1G50030 AT1G63020 AT2G23740 AT2G28290 AT3G49600 AT4G35800 AT5 |

G47010

| | |
|---|---|
| #> | ------- |
| #> | $AT1G50030 |
| #> | GRSeqset object with 17748 ranges and 1 metadata column: |
| #> | seqnames ranges strand \| seq |
| #> | <Rle> <IRanges> <Rle> \| <character> |
| #> | [1] 1 18522248 + \| A |
| #> | [2] 1 18522249 + \| A |
| #> | [3] 1 18522250 + \| C |
| #> | [4] 1 18522251 + \| A |
| #> | [5] 1 18522252 + \| A |
| #> | ... ... ... ... . ... |
| #> | [17744] 1 18539991 + \| T |
| #> | [17745] 1 18539992 + \| T |
| #> | [17746] 1 18539993 + \| T |
| #> | [17747] 1 18539994 + \| T |
| #> | [17748] 1 18539995 + \| T |
| #> | ------- |
| #> | seqinfo: 1 sequence from an unspecified genome; no seqlengths |
| #> | |
| #> | ... |
| #> | <6 more GRSeqset elements> |
| #> | ------- |

Next, the binary signal on the gene regions is retrieved with function getSignalAtRegions. The methylation signal is provided for the three genes in five different Col-0 samples and five msh1 mutants (dwarf) samples. Only DMPs are included.

In different samples, methylation can be located in different positions, so there is a set complete.region=TRUE to retrieve information from the entire regions and zero_signal=TRUE to permit positions with no signal.

```
at_signal <- getSignalAtRegions(signs = at_meth,
  regions = at_genes,
  minSigCount = 0L,
  signalCol = NULL,
  signal.direction = FALSE,
  zero_signal = TRUE,
  complete.region = TRUE,
  id_col = 1L,
  ignore.strand = TRUE)
```

This dataset is included with the R package:

data (at_signal)

### Example Results

Additional results supporting our heuristic for identification of methylation motifs in the Arabidopsis thaliana methylome is provided below:

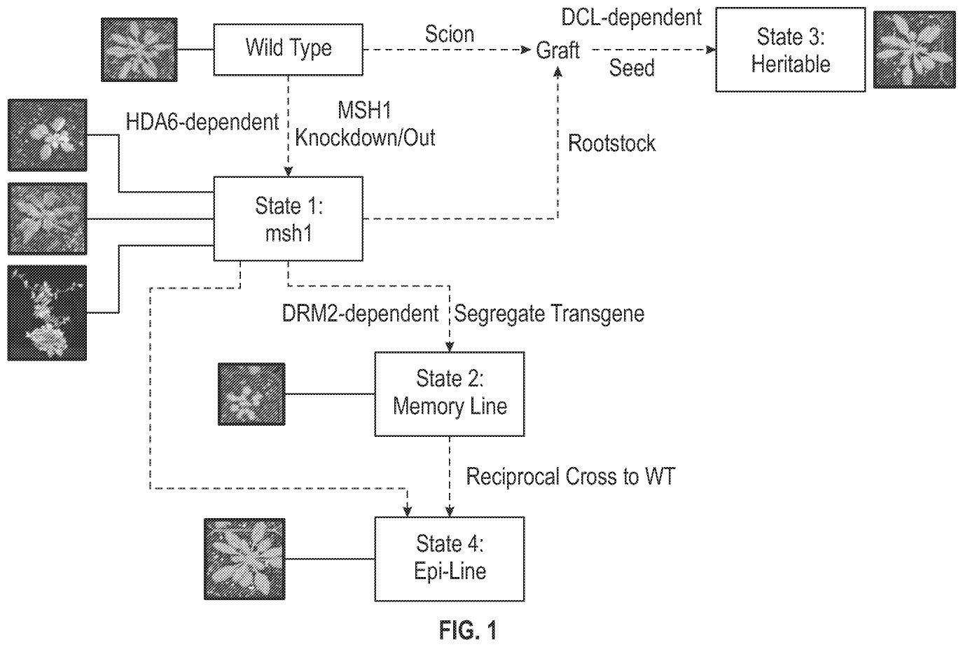

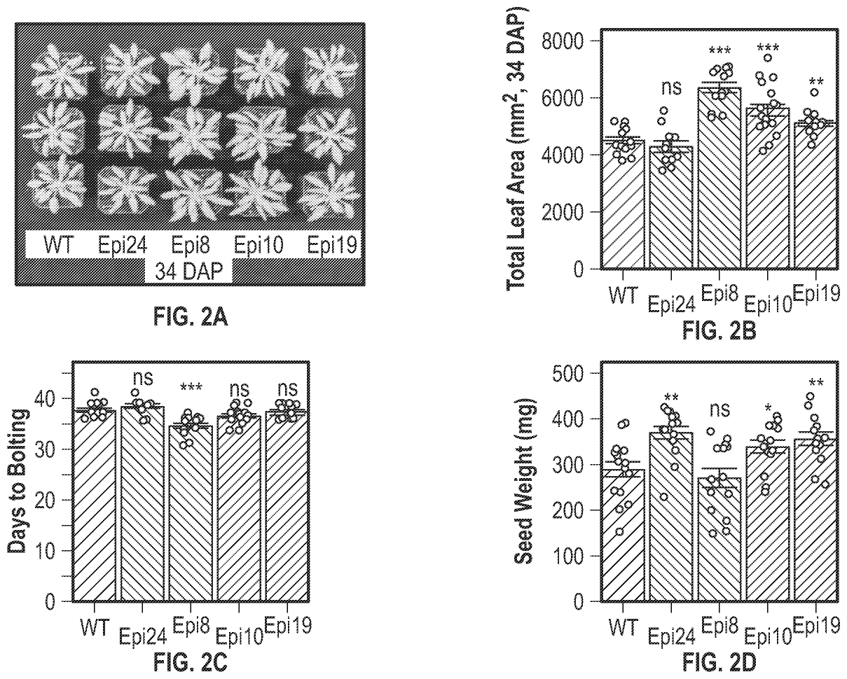

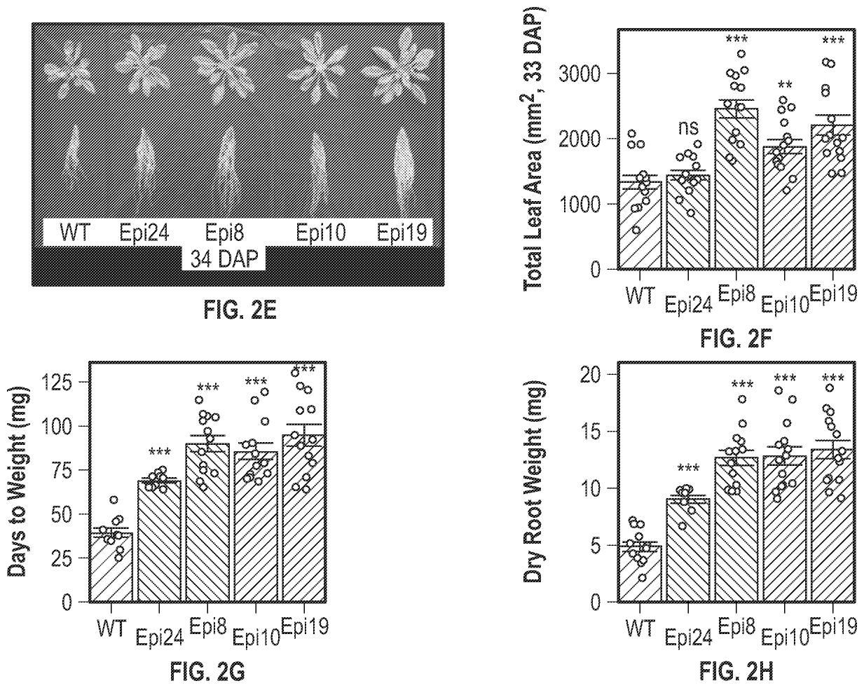

msh1 epi-lines comprise a distinct nongenetic state based on phenotype. Manipulation of the msh1 mutant leads to four distinct phenotypes, with states 1 and 2 characterized by slowed growth, delayed flowering and persistent stress response, and states 3 and 4 producing enhanced growth vigor and greater seed set over wild type (WT) (FIG. 1). State 4 results from direct or reciprocal crossing of Col-0 msh1 mutant (state 1) or memory line (state 2)×Col-0 WT and generation of epi-F2 and epi-F3 families in Arabidopsis. Similar results were obtained regardless of the direction of the cross. Progeny populations showed more variable distribution of growth-enhanced phenotypes within the F₂ generation than occurs in state 3 graft progeny, and individual epi-line populations could be categorized with either enhanced vegetative growth, greater seed set, or both (FIG. 2).

4 F3 populations were followed of cross-derived epi-lines. Epi 8 and Epi 24 were sibling lines from one WT×msh1 cross event, and Epi 10 and Epi 19 were sibling lines from a second WT×msh1 cross. All four F3 epi-lines showed uniform phenotypes within each population, but significant variation between the four populations (FIG. 2). Epi-line populations were increased in aboveground vegetative growth and underground root development (FIGS. 2A, 2E). Three of the populations, Epi 8, 10 and 19, had significantly higher total leaf area than the WT control (FIGS. 2B, 2F), and all four populations had higher dry leaf weight than WT (FIG. 2G). The four populations also showed higher dry root weight than WT (FIG. 2H).

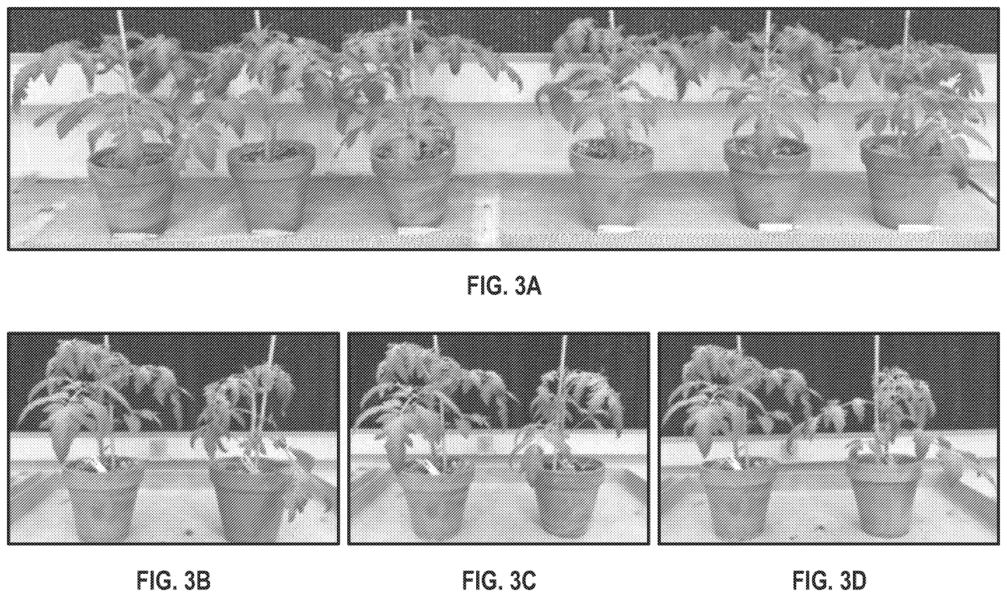

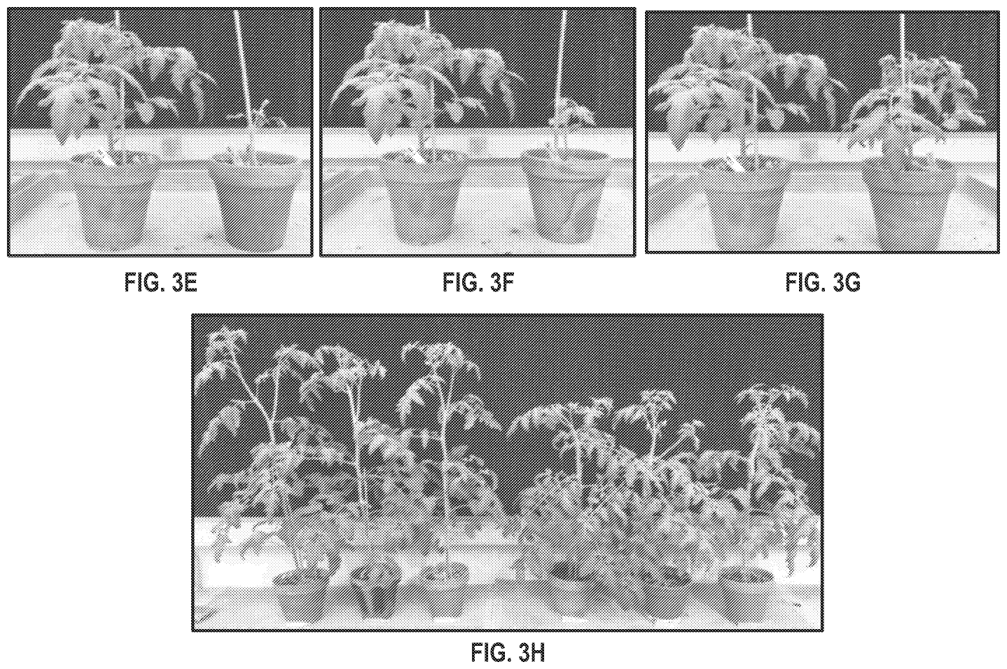

Enhanced reproductive growth occurred in the three epi-lines, Epi 8, 10, and 19, while Epi 8 showed early flowering (FIG. 2C). Importantly, Epi 8 and Epi 24, full-sib populations from the same original cross, showed distinct phenotypes. Epi 24 was highest in seed weight and lowest in leaf area among the epi-lines, while Epi 8 was highest in leaf area and lowest in seed weight. These observations indicate that growth vigor can vary in vegetative versus reproductive allocations within closely related populations. A similar phenomenon may occur in tomato epi-lines (FIG. 3), where sibling epi-line populations also differed in vegetative growth enhancement (FIG. 3H).

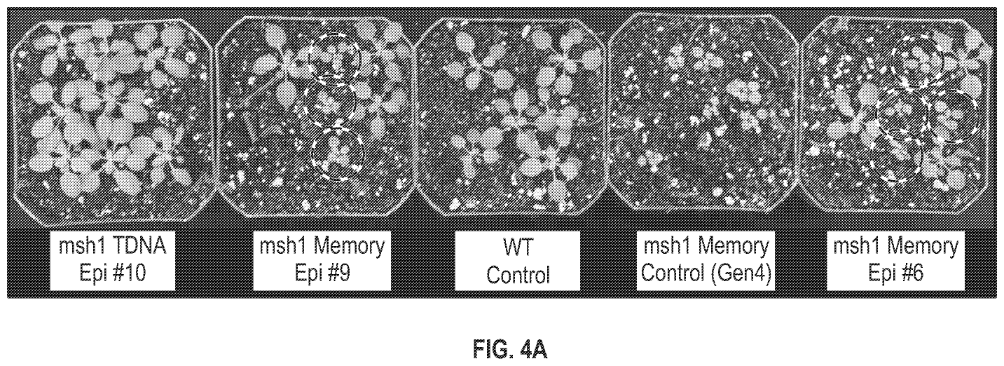

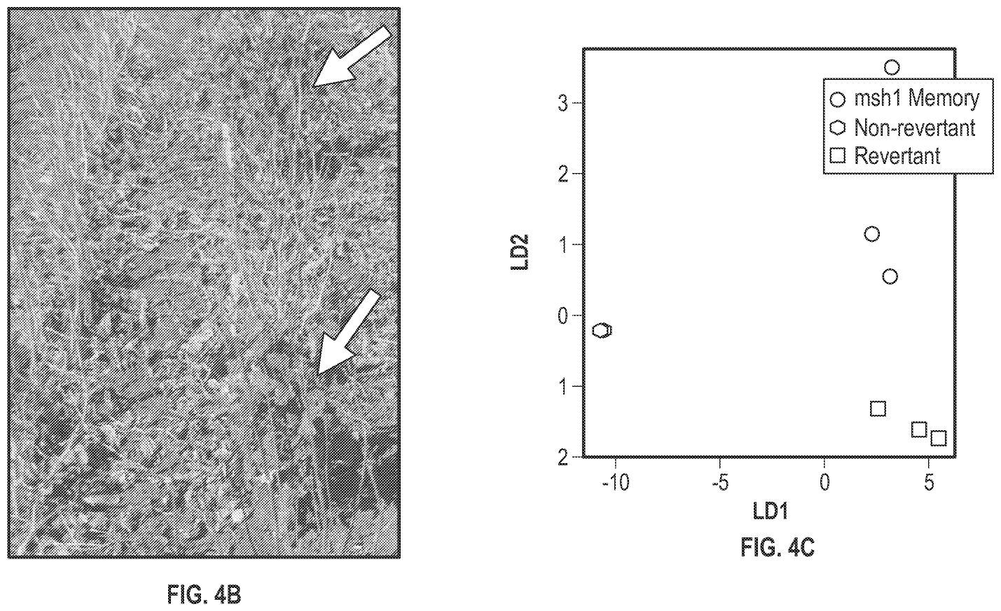

The epi-line phenotype receded back to wild type by the fifth or sixth (S5, S6) generation. Over these sequential generations, sporadic incidence of reversion to a condition resembling memory (state 2) phenotype (FIGS. 4A, 4B) was observed, characterized by reduced growth rate, altered leaf morphology and enhanced stress response. This reversion has not been observed in graft progeny. Reversion frequencies varied among populations and occurred across species (Table 1).

TABLE 1
Reversion frequencies within epi-lines

| | | |
|---|---|---|
| Lines tested* | Population | Reversion frequency |

Soybean

| | | | |
|---|---|---|---|
| WT × n (memory) | epi-F2 | 1/66 | (1.5%) |
| WT × i (memory) | epi-F2 | 1/60 | (1.7%) |
| WT × e (memory) | epi-F2 | 1/62 | (1.6%) |
| WT × n (memory) | epi-F2 | 2/30 | (6.7%)** |

Arabidopsis

| Epi 9 (memory) | epi-F3 | 20/106 | (18.87%) |
|---|---|---|---|
| Epi 6 (memory) | epi-F3 | 17/111 | (15.32%) |
| Epi 10 (TDNA) | epi-F3 | 0/77 | |
| msh1 memory | Gen 4 | 35/35 | (control) |
| WT Col-0 | | 0/55 | (control) |

*n, i, and e refer to mild (normal), intermediate, and extreme phenotype of msh1 memory line used in cross to generate epi-line populations. (TDNA) refers to msh1 TDNA insertion mutant used in cross as control.

Plant features of the four msh1-derived states in Arabidopsis recapitulated in tomato (FIG. 3) and soybean lines used in the study. The cross-species similarities included epi-line reversion (FIG. 4; Table 1). What is now recognized as epi-line reversion was also previously reported as instability in msh1-altered sorghum. Observation of the reversion phenomenon across different species implies that the epi-line state 4 and memory state 2 conditions are closely related. Reversion occurred only in epi-line populations deriving from crosses to an msh1 memory line (state 2) parent, but not with msh1 null mutant (state 1) as parent (Table 1). One interpretation of this observation is that msh1 signal is weaker in the memory line than null mutant, leading to a less stable epi-line outcome.

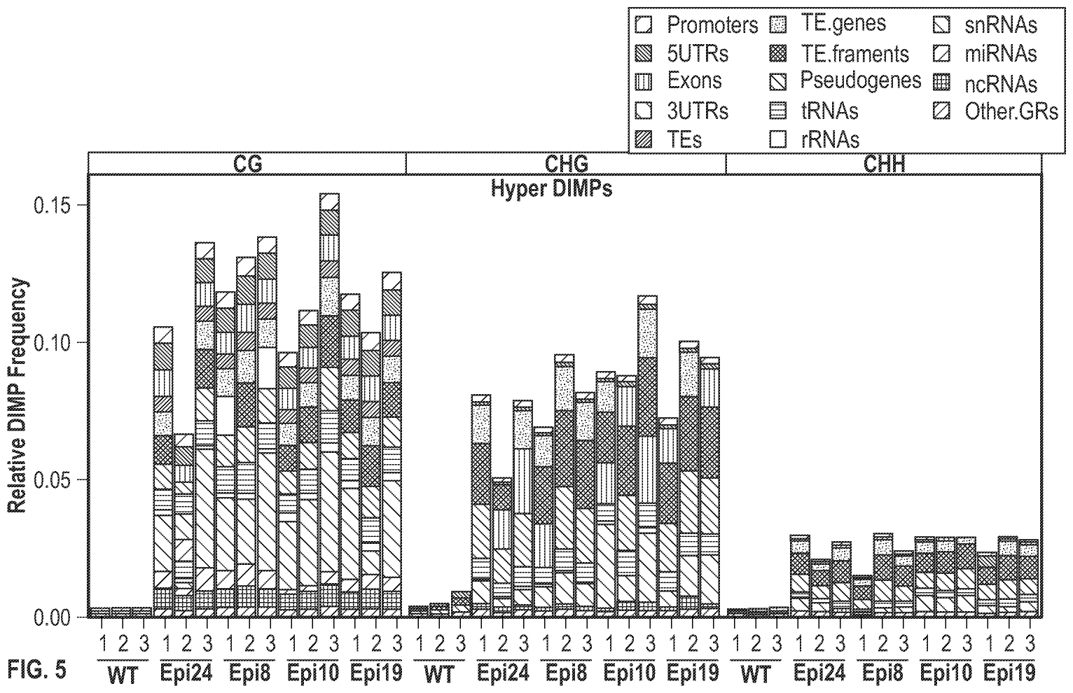

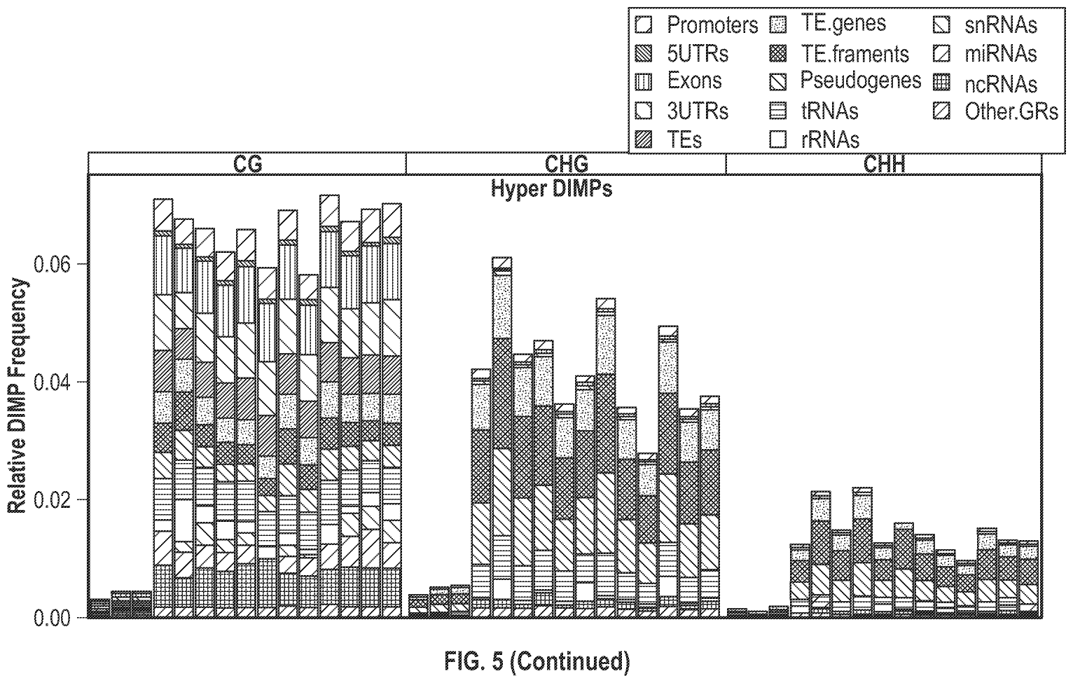

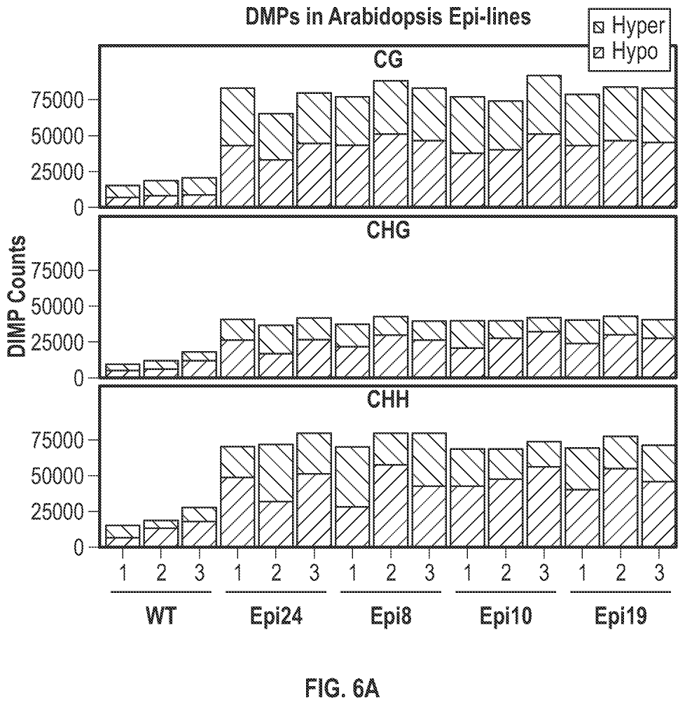

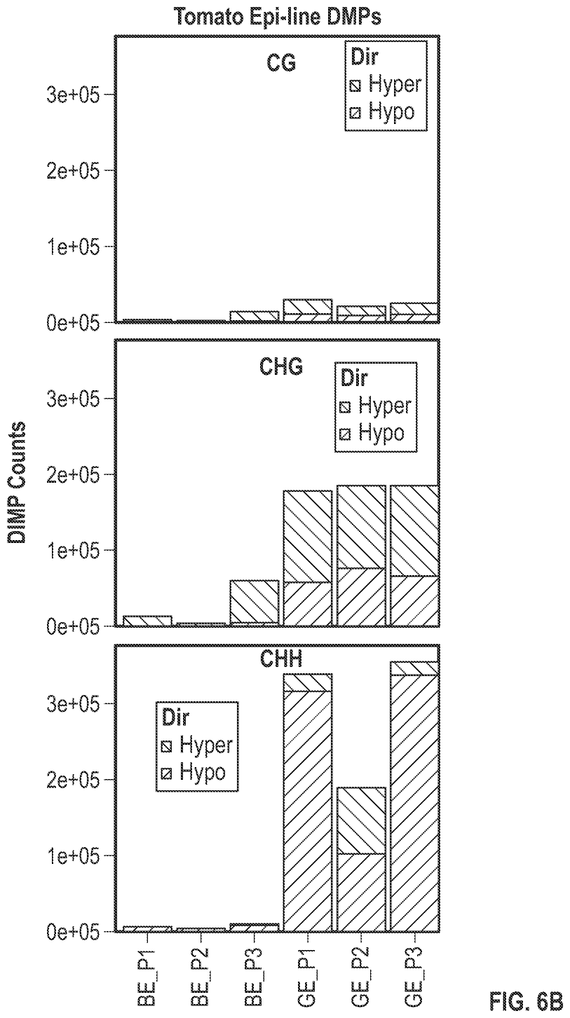

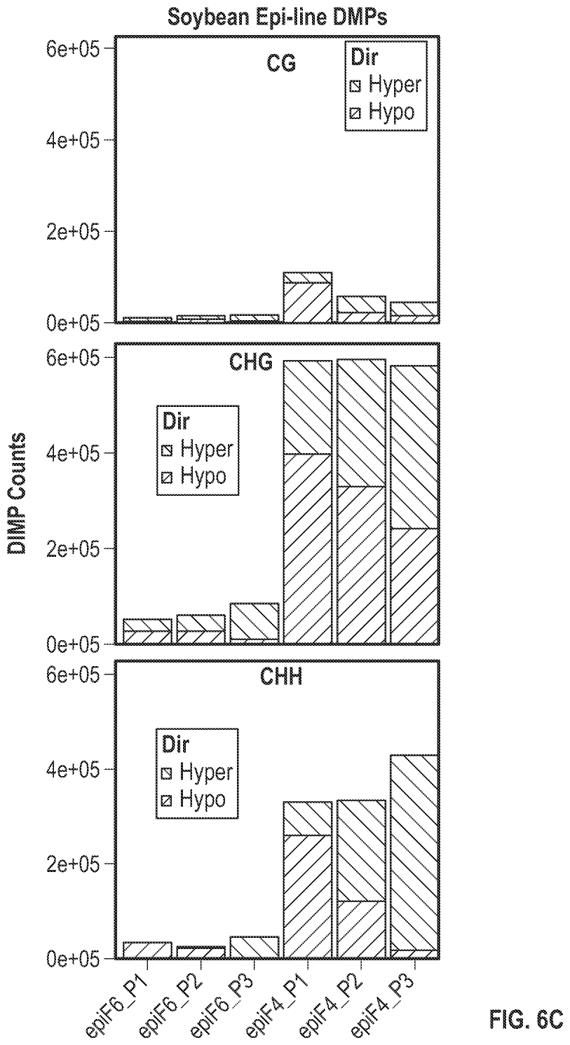

The msh1 states 1 to 4 comprise discrete epigenetic phases by whole-genome methylome analysis. Significant changes in DNA methylation were detected in the four Arabidopsis epi-lines (F3), with gene-associated changes predominantly in CG context (FIG. 5). Similar DNA methylation changes occurred in Arabidopsis, tomato and soybean epi-lines, displaying variable hyper and hypomethylation in CHH context within and between epi-lines in the three species (FIG. 6). In the soybean Epi-F4 vs Epi-F6 comparison, where Epi-F4 lines were enhanced in growth vigor that declined to wild-type phenotype by the F6 generation, potential growth vigor-associated DNA methylation changes (FIG. 6C) could be identified.

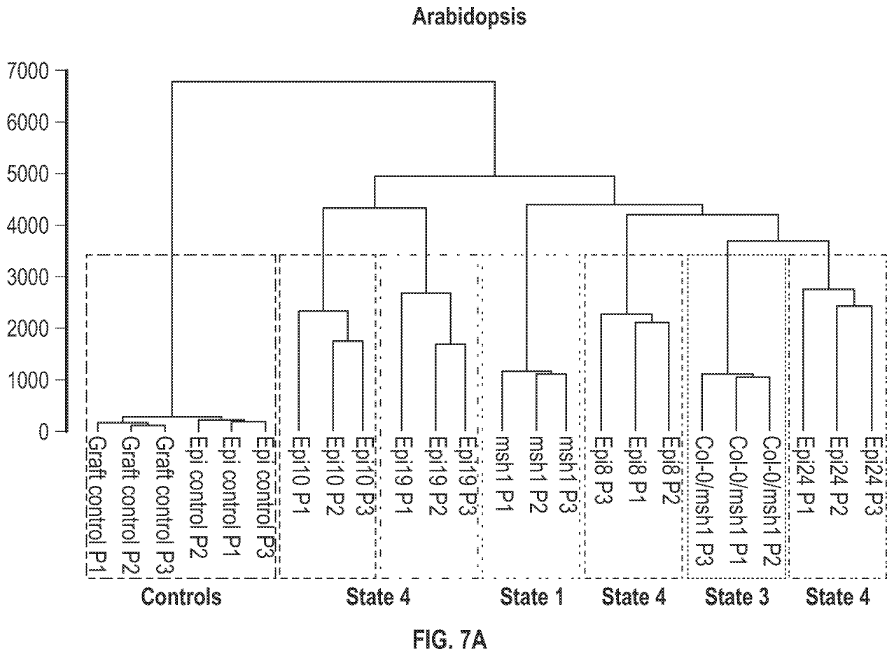

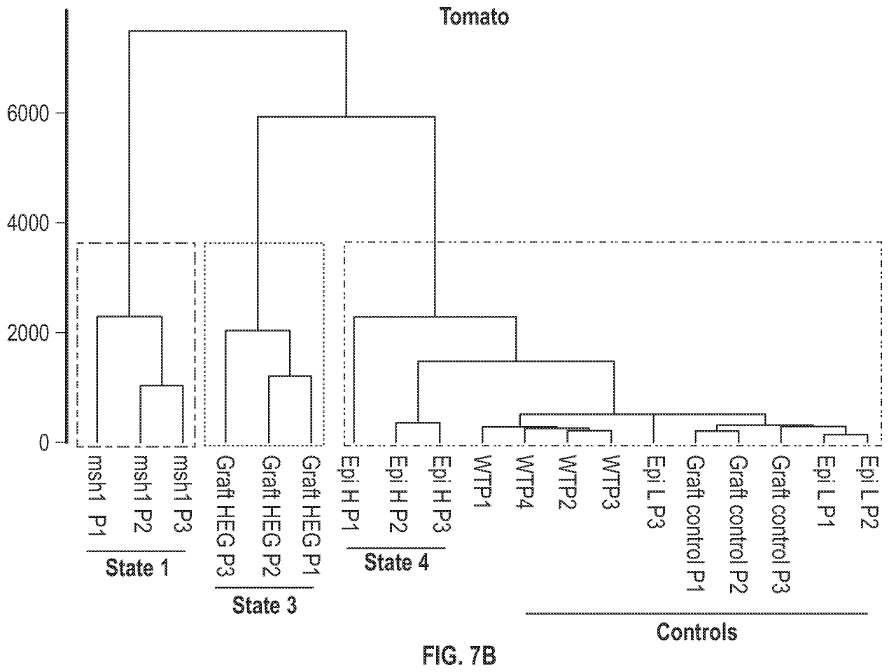

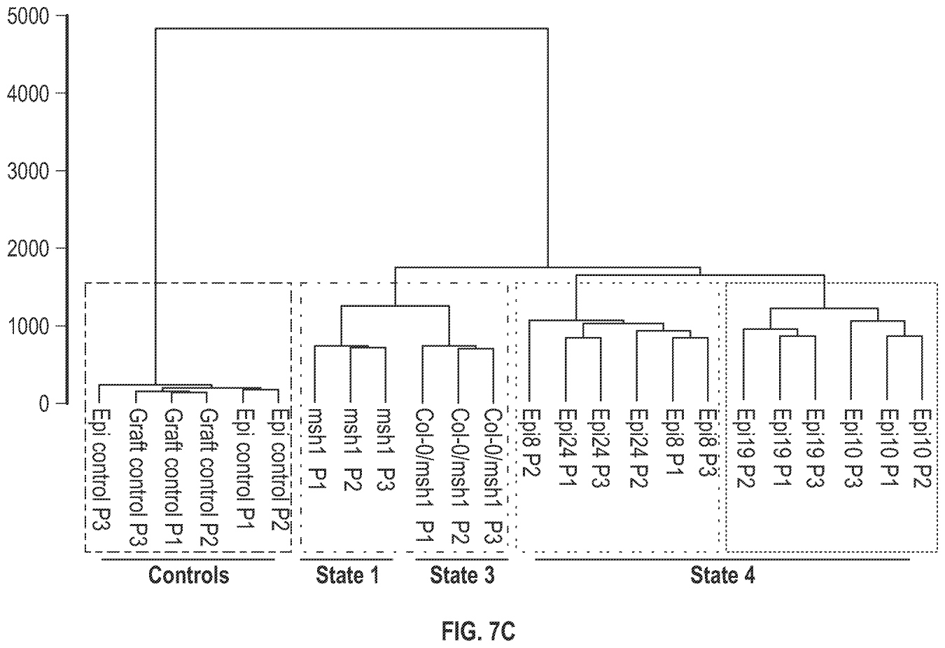

To estimate the relationship of genic differential methylation with changes in plant phenotype, a high-resolution methylome analysis was used. The procedure incorporates signal detection and machine learning for discrimination of high-probability, treatment-associated methylation changes within gene regions. Hierarchical clustering of methylome data from individual plants from all four states in Arabidopsis used methylation level changes (computed as Hellinger divergence) at differentially methylated positions (DMPs) in gene regions. The result showed clustering of individual plants (biological replicates) from the same population, with plants from different states separated to 6 branches distinct from WT control groups (FIG. 7). Despite originating from the same cross, Epi 8 and Epi 24 separated to two subgroups (FIG. 7A), in seeming agreement with their distinct phenotypes (FIG. 2). The data suggested that Epi 24 genic DNA methylation repatterning was more closely related to that of graft progeny (HEG; state 3) than to its full-sib Epi 8 (FIG. 7A). Epi 10 and Epi 19 also formed two closely related clusters consistent with their phenotypic similarity. In contrast, hierarchical clustering of transposable element-associated DMPs in the four epi-lines produced outcomes in keeping with lineage, so that Epi 8 and Epi 24 now clustered together, as did Epi 10 and Epi 19 within a single sub-cluster, and both clusters separated from the graft progeny (HEG) state 3 (FIG. 7C).

Similar results emerged in hierarchical cluster analyses of tomato genic methylome data. Tomato msh1 mutant (state 1), HEG (state 3) and epi-line datasets (state 4) formed three branches, consistent with distinctive gene methylation effects (FIG. 4b). Two epi-lines that differed in plant growth vigor phenotype, Epi High (H; enhanced in biomass over WT) and Epi Low (L; comparable in biomass to WT), separated to two subgroups within the same main cluster (FIG. 7B). The observations indicate that gene-associated methylation repatterning can be discerned to reflect plant phenotype among lines predicted to be genetically uniform.

Methylation data support epi-line revertants as more closely related to msh1 memory state 2. Epi-line (state 4) revertant samples in Arabidopsis were included in methylome analyses to test for evidence of methylation repatterning in the revertant versus non-revertant full-sib samples. Principal component with linear discriminant analysis (PCA-LDA) of these datasets using Hellinger divergence produced distinct clustering of non-revertant from revertant individuals deriving from the same progeny population. The epigenomic features distinguishing revertant and non-revertant full-sib progeny presumably arose spontaneously. Revertant methylome datasets showed a closer relationship with data from msh1 memory state 2 (FIG. 4C). These msh1 memory plants were derived from an independent population. A relationship of the revertant methylome profile with msh1 memory would be consistent with their demonstrated similarity in plant phenotype (FIG. 4). These results support previous indications of msh1 memory as distinctive in methylome features¹⁸ and confirm low-frequency conversion of epigenetic state 4, enhanced in growth vigor, to something resembling state 2, a persistent stress response phenotype.

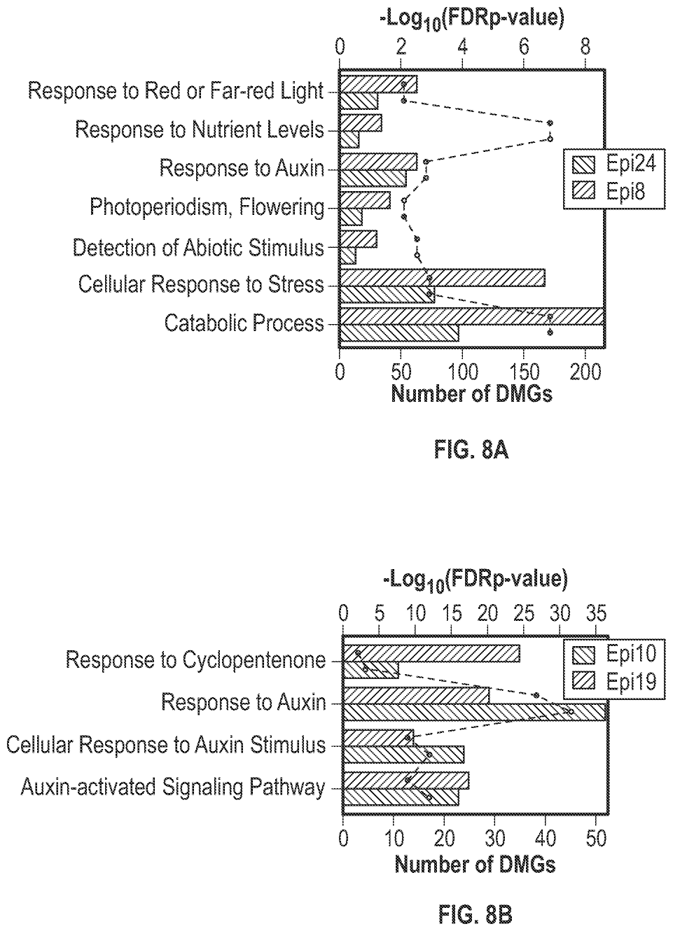

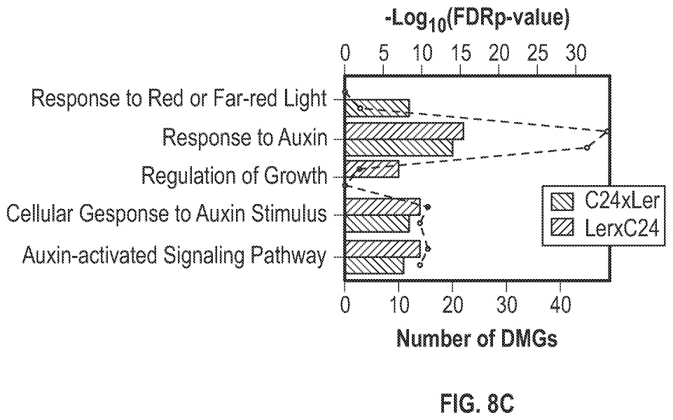

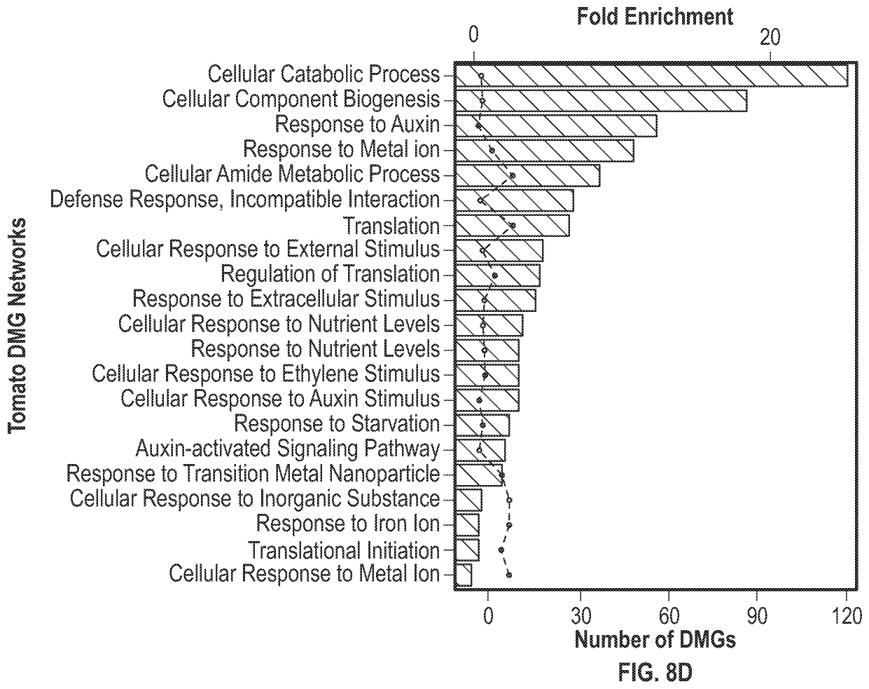

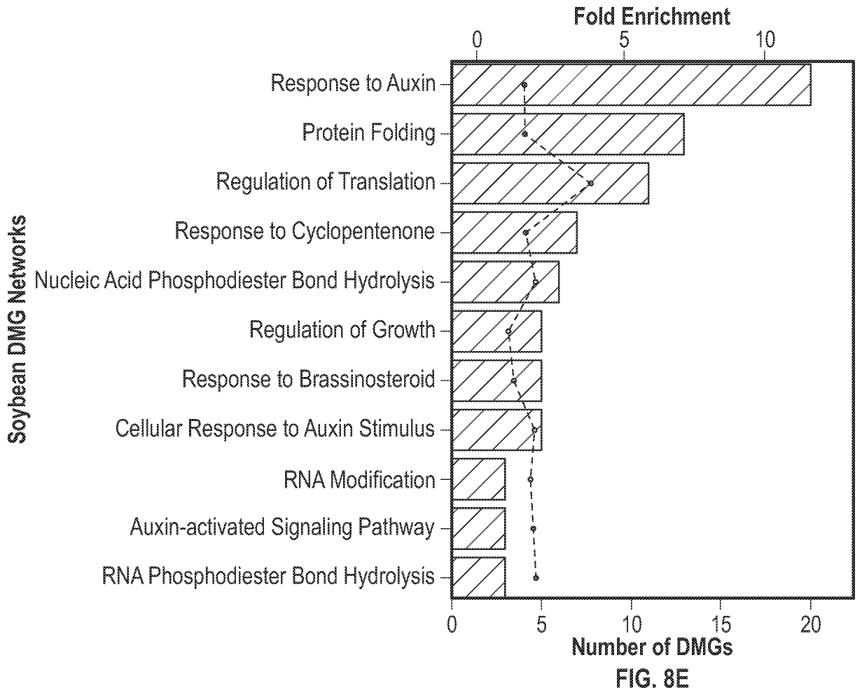

Epi-line methylome datasets are altered in growth-related gene networks. Differentially methylated genes (DMGs) for each epi-line population were identified by applying generalized linear regression analysis (GLM) to test significance of the difference between group DMP counts (WT vs. epi-lines) in genes. This analysis identified 3204, 2860, 3208 and 2797 DMGs in Epi 8, Epi 10, Epi 19 and Epi 24, respectively. To investigate coincidence of DMG functional relationships in each population, a gene network-based enrichment analysis was conducted. DMGs from the different epi-lines shared enrichment for specific functional networks. For example, the full sibs Epi 8 and Epi 24 shared common enriched networks for response to auxin, response to red or far-red light, response to nutrient levels, photoperiodism, detection of abiotic stimulus, response to stress, and catabolic process (FIG. 8A), while Epi 10 and Epi 19 shared enriched networks for response to cyclopentenone, response to auxin, cellular response to auxin stimulus, and auxin-activated signaling pathway (FIG. 8B). This outcome reflects a non-random quality of DMP datasets, implying that methylation machinery-targeted gene loci and their respective networks can be identified.

Methylome data previously reported for the Arabidopsis F1 heterotic cross of ecotypes C24 and Ler was analyzed using the methylome analysis methods that were applied to the msh1 datasets. The enriched networks emerging from F1 hybrid (C24×Ler, and Ler×C24) data showed remarkable conformity to what was identified in msh1-derived epi-line data, emphasizing pathways for response to red or far-red light, response to auxin, regulation of growth, cellular response to auxin stimulus and auxin-activated signaling pathways (FIG. 8C). DMG overlap between hybrid data and epi-lines ranged from 47% overlap with Epi 10 to 54% overlap with Epi 8. This apparent alignment of methylome-identified gene pathways between the heterotic hybrids and msh1 epi-lines supports linkage of phenotype with methylome repatterning and associates enhanced vigor with changes in auxin signaling. The observations were also consistent with profiles of DMG-derived enriched networks in tomato and soybean epi-line datasets, which identified prominent auxin-related networks (FIGS. 8D-8E). The soybean studies emphasized phenotype changes, where Epi-F4 lines were measurably enhanced in growth vigor that, by the Epi-F6 generation, diminished back to wild type growth. Consequently, the methylome features were analyzed that distinguish the Epi-F4 from Epi-F6 datasets as a direct comparison to discriminate vigor-associated methylome features within a single lineage.

Examination of gene expression changes in Arabidopsis epi-line populations involved sampling three different tissues for RNAseq analysis. Epi 8 and Epi 10 were contrasted with wild type for differential gene expression in leaf tissues, by similar sampling to that done for methylome studies, and Epi 8 and Epi 24 were additionally compared by analysis of floral stem and root tissues. From leaf tissues, 1884 differentially expressed genes (DEGs) were identified in Epi 8 and 992 in Epi 10 relative to wild type. Epi 8 and Epi 24 analysis revealed 1991 DEGs from floral stem in Epi 8 and 1650 for Epi24 and, from root, 1133 DEGs in Epi 8 and 1111 in Epi 24 relative to wild type.

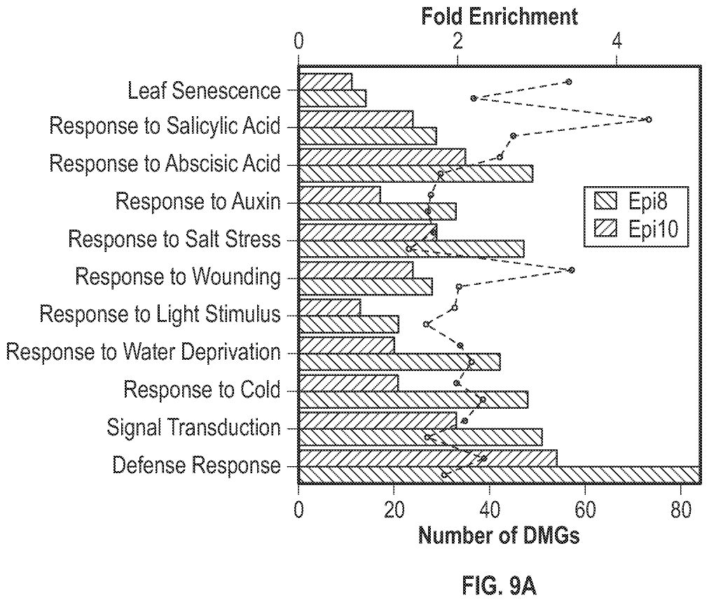

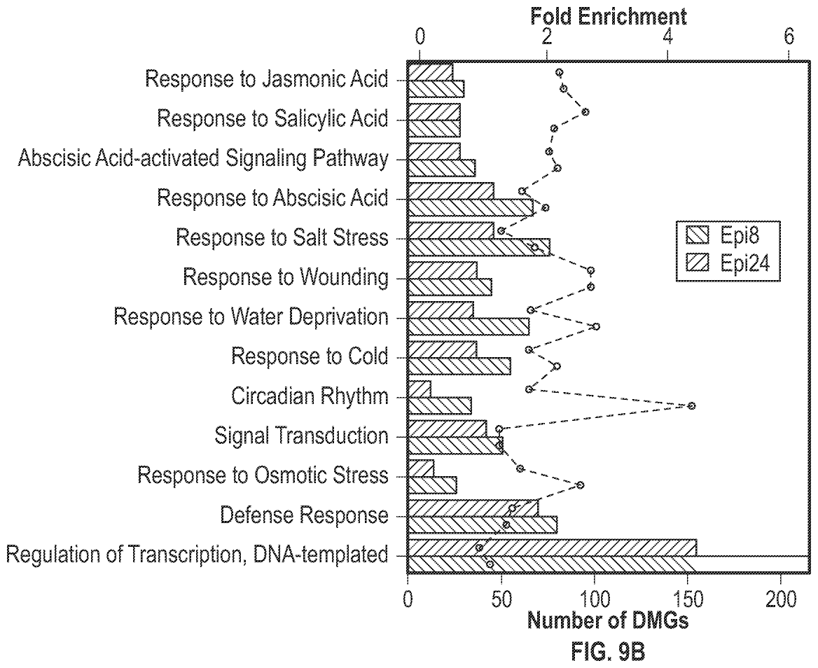

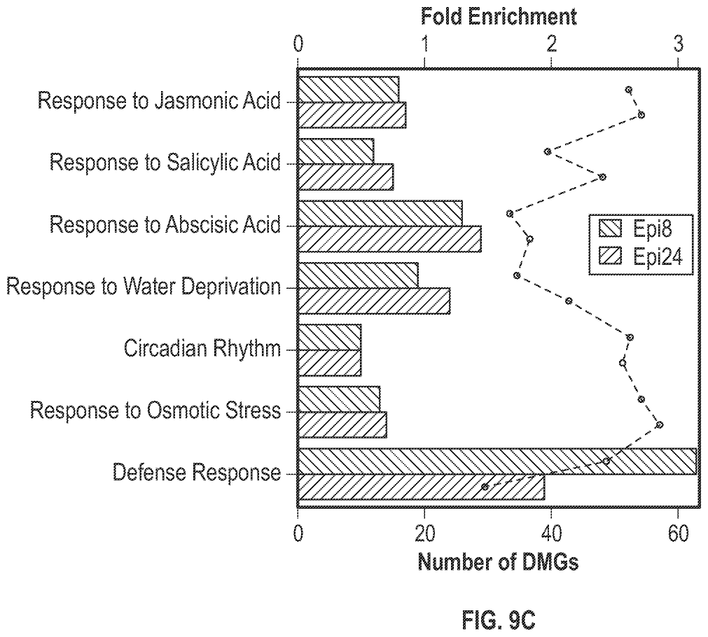

Network enrichment analysis of derived DEG datasets showed shared pathways altered in response to msh1 effects in leaf, floral stem and root tissues. The most enriched of these involved abiotic and biotic stress responses, with circadian rhythm- and phytohormone response-related networks also significantly enriched in the three tissues. Regulation of transcription was prominent specifically in floral stem, where MSH1 accumulates (FIG. 9). These outcomes appeared consistent with epi-line DMG results (FIG. 8), such that integration of DEG and DMG datasets served to amplify the msh1-associated network enrichments relative to either dataset alone.

Differential methylation and expression analysis identified central gene hubs for the epi-line state. To investigate the interaction of DMG and DEG datasets in epi-lines involved inputting DMG and DEG data to Cytoscape to construct protein-protein interaction (PPI) maps, followed by K-means cluster analysis to identify putative core networks carrying central gene hubs. A K-means cluster machine learning algorithm uses betweenness centrality, closeness centrality, average shortest path length, clustering coefficient, degree, and eccentricity as parameters, allowing the identification of clusters that contain the most centralized nodes (proteins) in the PPI network.

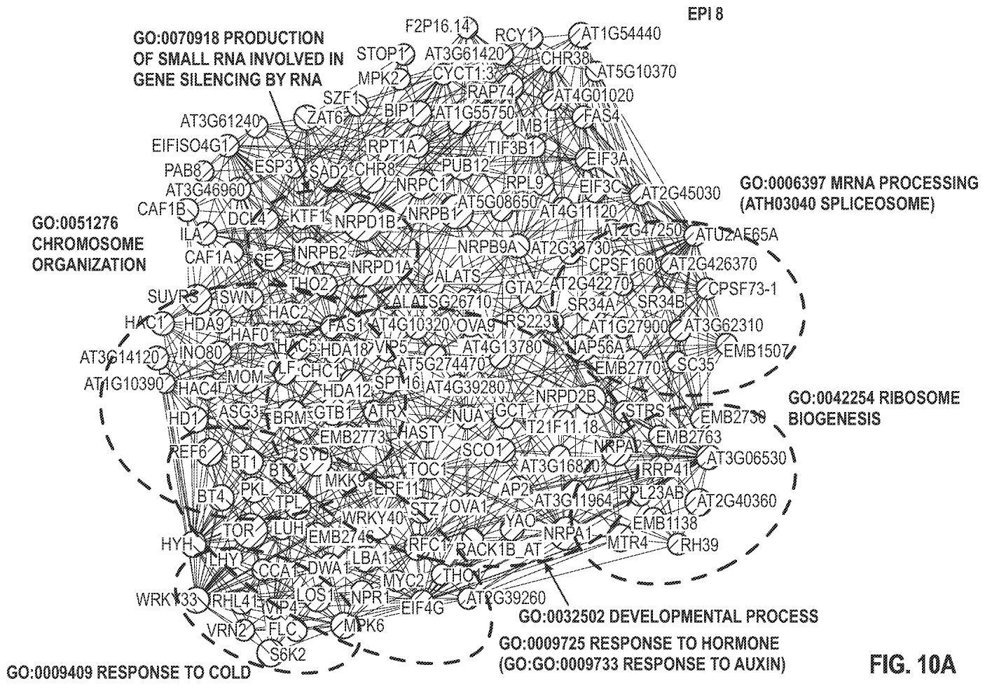

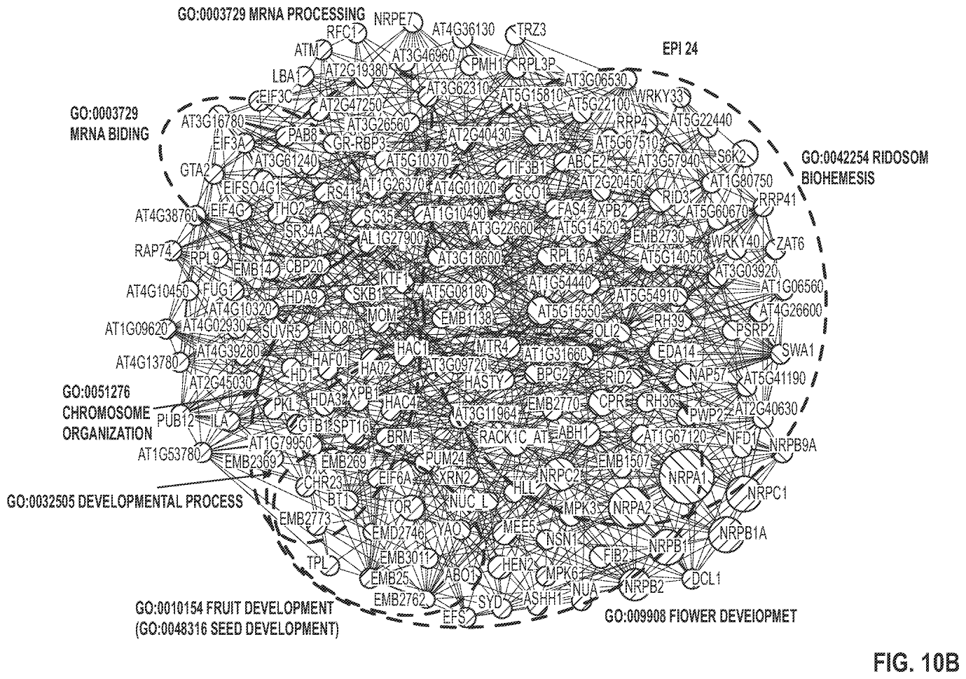

In Arabidopsis Epi 8, a total of 3647 unique loci from DMGs and DEGs were used in the analysis to yield a PPI network formed by 430 genes. Functional enrichment analysis of these putative hub genes with the STRING database functional enrichment tool revealed a PPI network of 153 hub genes and associated functional networks (FIG. 10A). These core networks included developmental process, response to hormone (particularly auxin), response to cold, chromosome organization, mRNA processing, spliceosome, and ribosome biogenesis. Comparison to Epi 24, with a total of 3523 unique loci from DMGs and DEGs forming a much larger PPI network of 346 genes, showed that several of these same processes were represented (FIG. 10B). However, Epi 24 analysis also revealed more prominent changes in development-related gene expression and, most notably, a robust and overlapping effect in ribosome biogenesis-related gene expression (FIG. 10B). Ribosome biogenesis is integral to growth response. Because Epi 8 and Epi 24 are full sibs from the same cross, these differing features are predicted to relate to their distinct phenotypes, with Epi 8 enhanced in vegetative growth and Epi 24 showing significantly increased reproductive vigor (FIG. 2).

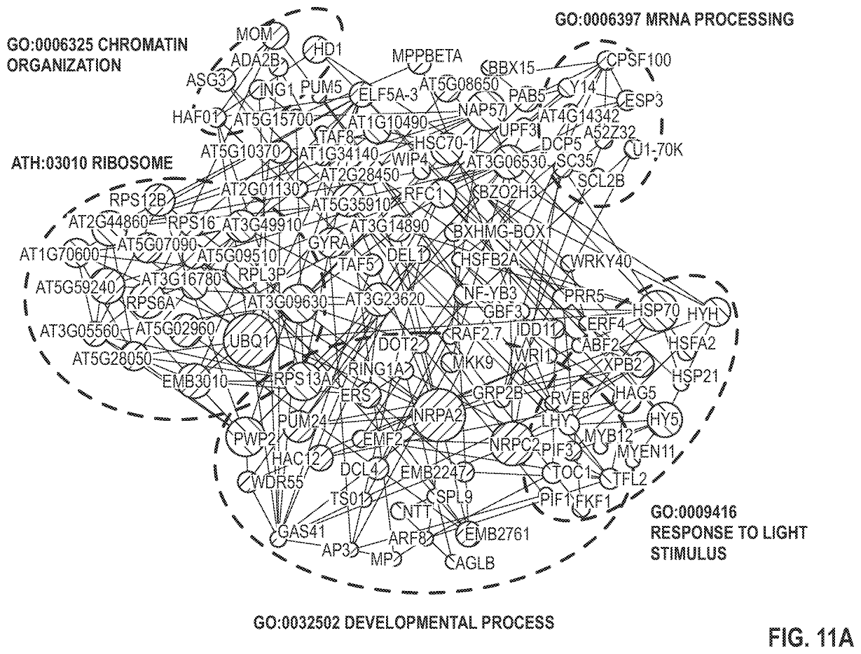

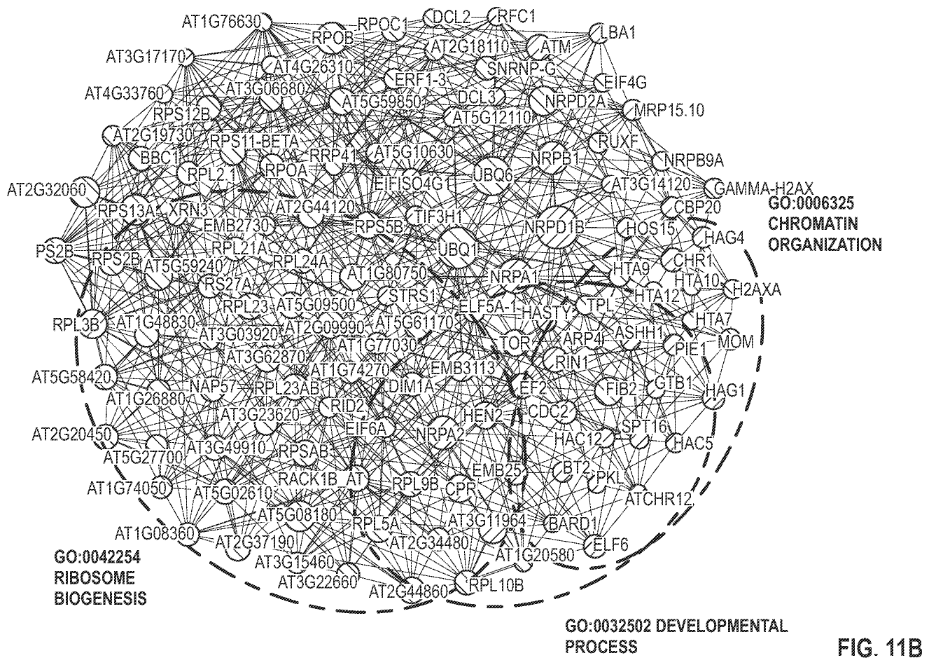

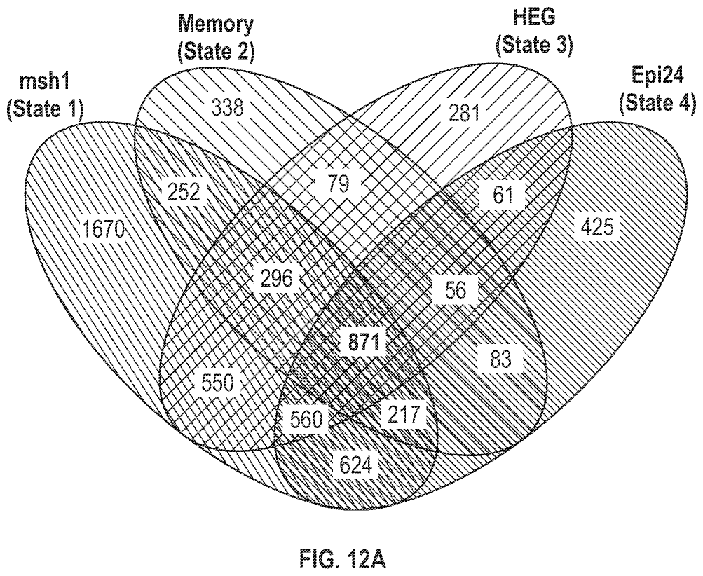

Analysis of soybean epi-line data involved assignment of the Arabidopsis ortholog to each identified soybean gene with BLASTP and obtaining a soybean epi-line PPI network of 109 core hub genes and their functional networks (FIG. 11A). Resulting enriched networks included developmental process, ribosome, chromatin organization, mRNA processing, and response to light stimulus. These results indicated cross-species overlap in identified pathways (FIG. 11A), so that msh1-directed effects led to parallel phenotype outcomes in different species via similar networks. Annotated gene overlap between soybean and Arabidopsis for core hub genes was 16% (18 genes).

To assess the relationship of PPI network output for epi-lines to graft-derived HEG, the core hub PPI network was constructed for the tomato HEG (state 3) phenotype. The network contained ribosome biogenesis, developmental process, and chromatin organization (FIG. 11B). Identified core hub proteins shared in epi-line and HEG datasets from soybean, Arabidopsis and tomato, included TOR, MOM, RFC1, UBQ family, NRPD family, and HAC family proteins. While there was cross-species conservation of several overlapping hubs, detailed cross-species comparisons were limited by disparities in gene annotation.

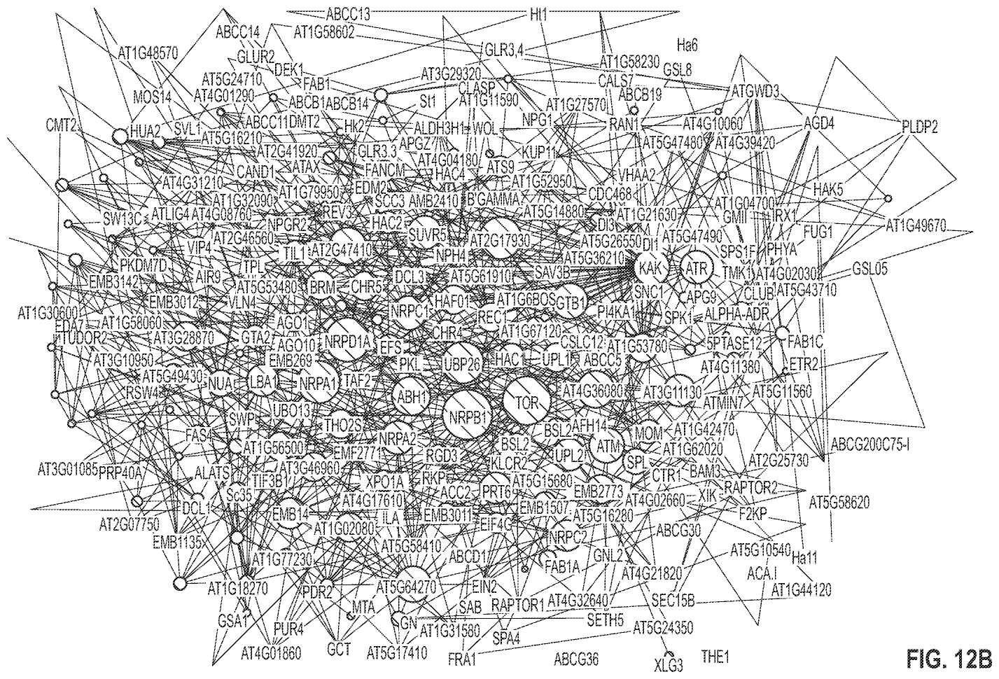

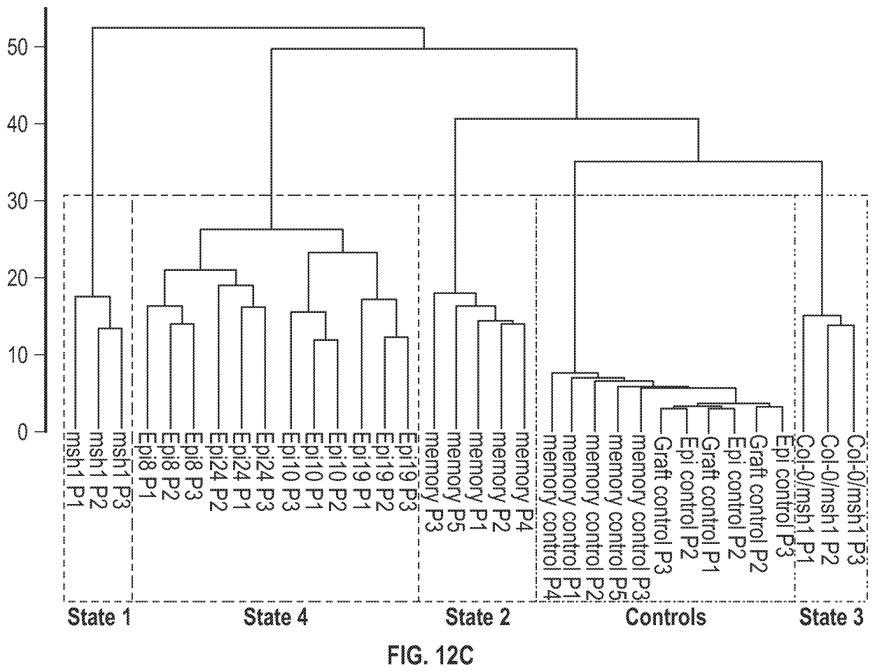

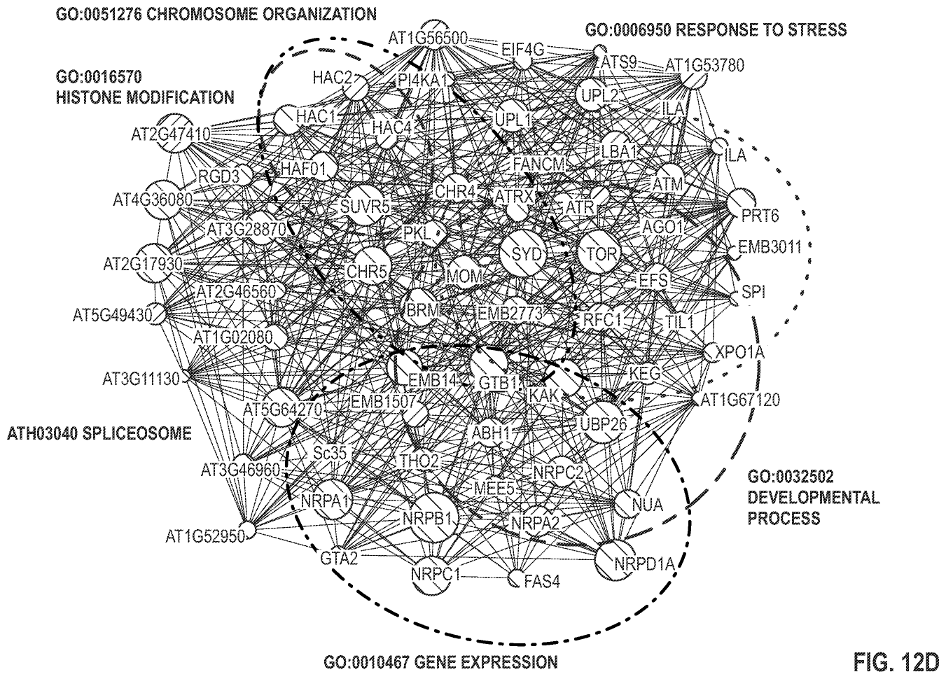

Epi-state comparisons in Arabidopsis reveal conserved msh1 epigenome targets within biologically meaningful gene networks. To investigate the relationship of genic methylation repatterning among the four distinct msh1-derived states, DMG overlap was assessed. FIG. 12A shows that 871 DMGs were shared among the four msh1 states, comprising 17.6% of msh1 (state 1), 39.7% of memory (state 2), 31.6% of graft progeny (HEG, state 3) and 31.1% of epi-line (state 4, Epi 24) DMGs. The overlap established a conserved msh1 ‘core’ DMG dataset (FIG. 12A). Each state was also classified by state-specific DMGs, accounting for 33.8% in state 1, 15.4% in state 2, 10.2% in state 3, and 15.2% in state 4 (FIG. 12A). Of the 871 ‘core’ msh1-associated DMGs, 531 (61%) could be placed within known networks.

Components of the RdDM pathway were shown to be necessary for induction of msh1 state 1, transition from state 1 to state 2, and generation of state 3 following grafting. Methylome datasets that contrasted msh1 versus dcl2/dcl3/dcl4/msh1 quadruple mutant (state 1) or graft progeny from Col-0/Col-0ₘₛₕ₁ versus Col-0/Col-0_{dc11/dcl3/dcl4/msh1} grafts (state 3) served to catalog DMGs as RdDM (sRNA)-dependent. These subtractive datasets confirmed that 674 (77%) of the 871 core DMG loci were predicted to be DCL2,DCL3,DCL4-dependent by obtaining the overlap between the 871 DMG core dataset and the msh1 vs dcl2/dcl3/dcl4/msh1 dataset. FIG. 12B shows the PPI core hub network of DMGs with those predicted as DCL2,DCL3,DCL4-dependent having crosshatching. These outcomes supply evidence of a relationship between msh1-directed epigenetic effects and RdDM pathway influence on targeted methylome repatterning.

Whereas 871 core DMGs were shared among the four msh1 states, the methylation changes were discovered within these 871 loci also served to discriminate the four states. FIG. 12C shows the results of hierarchical clustering for individual plants from the four epigenetic states with separation to four distinct clades by using only DMP methylation information over the 871 msh1 core genes. Based on the PPI network for the 871 DMGs, K-means clustering was conducted to identify putative central hubs and their functional enrichment analysis. FIG. 12D shows the resulting network of 67 candidate hubs that are involved in response to stress, developmental (growth) process, gene expression, spliceosome, histone modification, and chromosome organization networks. Over 80% of the 67 hubs were predicted to be RdDM targets, with several central players in plant development and environmental stress response, such as the cell growth regulator TARGET OF RAPAMYCIN (TOR), the SWI2/SNF2-like, meristem identity gene SPLAYED (SYD), its close homolog BRAHMA (BRM), and the chromatin remodelers PICKLE (PKL) and PICKLE-RELATED 1 (PKR1/CHR4).

Transposable elements and sRNAs associate with candidate RdDM target loci among the 871 msh1 DMGs. The RdDM pathway is known to actively target transposable element (TE) sequences, prompting investigation of TE association with msh1-responsive loci. Looking at the 871 DMGs common between msh1 states, association of these loci was detected with TEs and sRNA (20-24 nt) clusters that was higher than genome-wide levels (61% DMGs within 2 kb of TE vs. 47% genome-wide; 77% DMGs within 2 kb of an sRNA cluster vs. 46% genome-wide). This enrichment for TE and sRNA cluster proximity increases further when the dcl2,dcl3,dcl4-sensitive DMGs (65% DMGs within 2 kb of TE; 81% DMGs within 2 kb of an sRNA cluster) were subset. Yet, when focused on the 67 hub DMGs derived by k-means clustering, only sRNA cluster enrichment was seen (49% of DMGs within 2 kb of TE; 72% DMGs within 2 kb of an sRNA cluster). FIG. 12D shows only TE-associated genes (7), only sRNA-associated genes (22), and genes associated with both TE and sRNA (26). Hence, although TEs may influence the methylation status of proximal genes and act as RdDM targets, sRNA cluster association regardless of TE proximity could define RdDM targets for the msh1 effect.

Some possible evidence was found of association between TE family and DMG proximity. Comparing the number of members in TE families between observed and expected revealed significant overrepresentation in L1 and Gypsy families and underrepresentation in the Helitron family, both in the 871 DMGs common between msh1 states and 674 DMGs sensitive to dcl2,dcl3,dcl4. Further investigation is needed to reveal any biological significance of these associations. Based on the various analyses, four criteria served to classify RdDM target loci in our study (FIG. 12D): Positive loci were (1) confirmed DMGs in all four msh1 states that (2) comprised putative network hubs based on k-means clustering, with genetic evidence for (3) dcl2,dcl3,dcl4-sensitivity, and/or (4) association with sRNA clusters.

Detailed methylation analysis of selected RdDM target genes in the four different nongenetic states reveals sequence motifs encompassing dcl2/3/4-sensitive DMPs. Annotations of the 67 candidate hub loci supported their relevance to phenotype effects observed in the four msh1 states. In addition to gene networks for altered gene expression and chromatin behavior, major overlapping networks appeared to reflect the observed transition between stress response and growth (FIG. 12D) that underpins msh1 and memory stress phenotypes versus the growth vigor of HEG and epi-line states. Methylation features of the 67 candidate hub loci that discriminate between the four msh1 states support a model of precisely targeted repatterning (FIG. 12C).

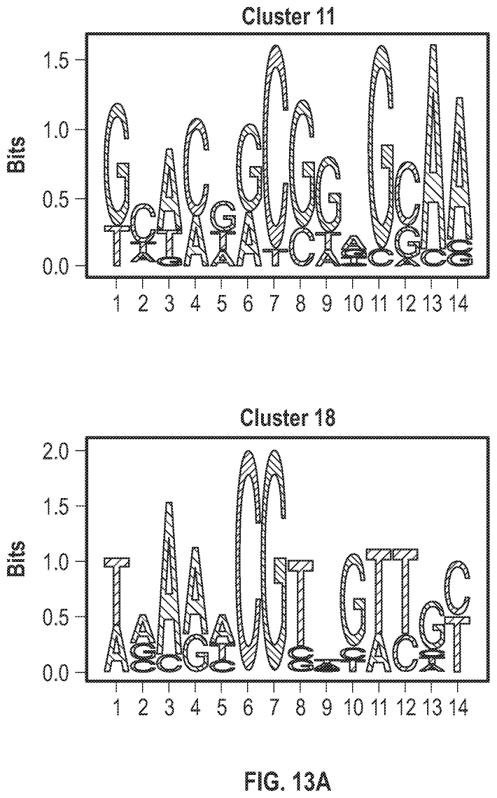

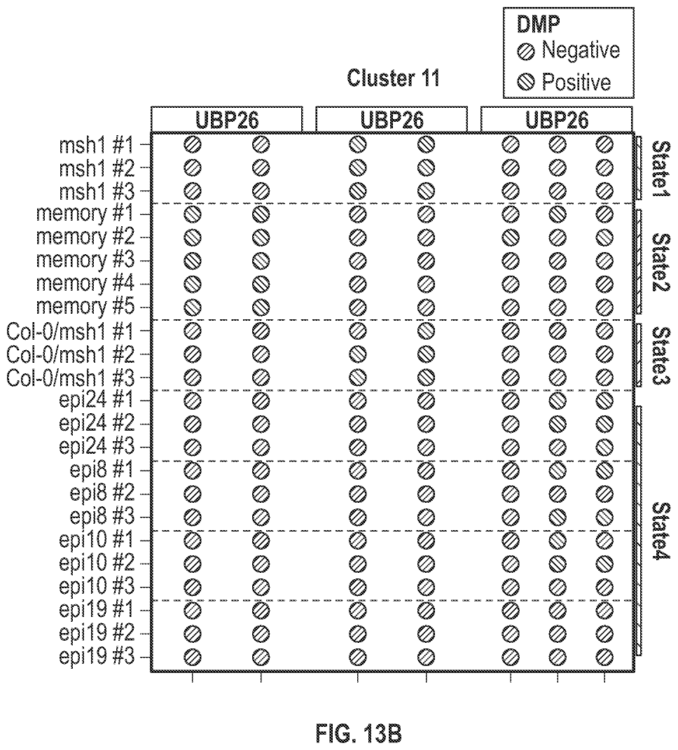

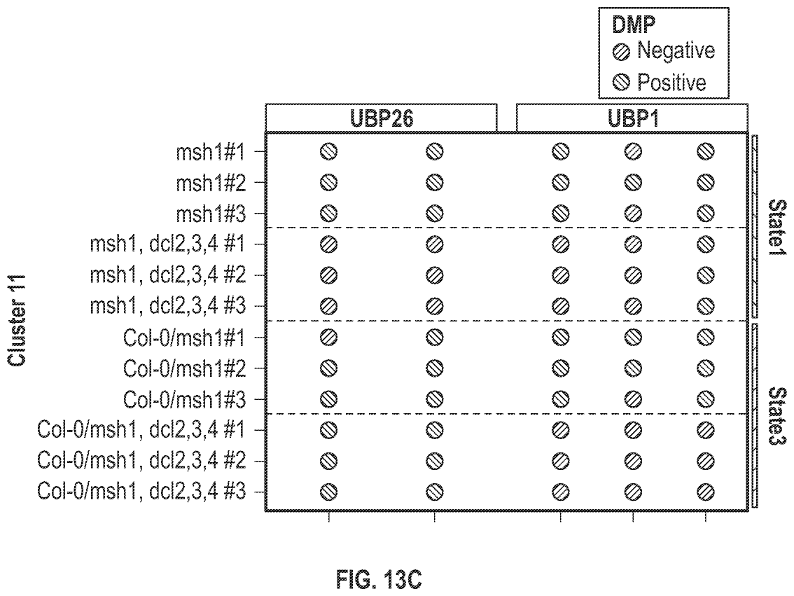

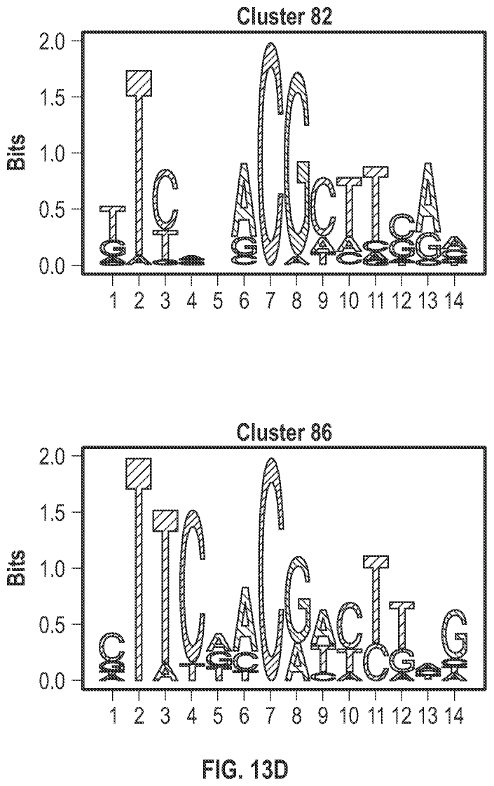

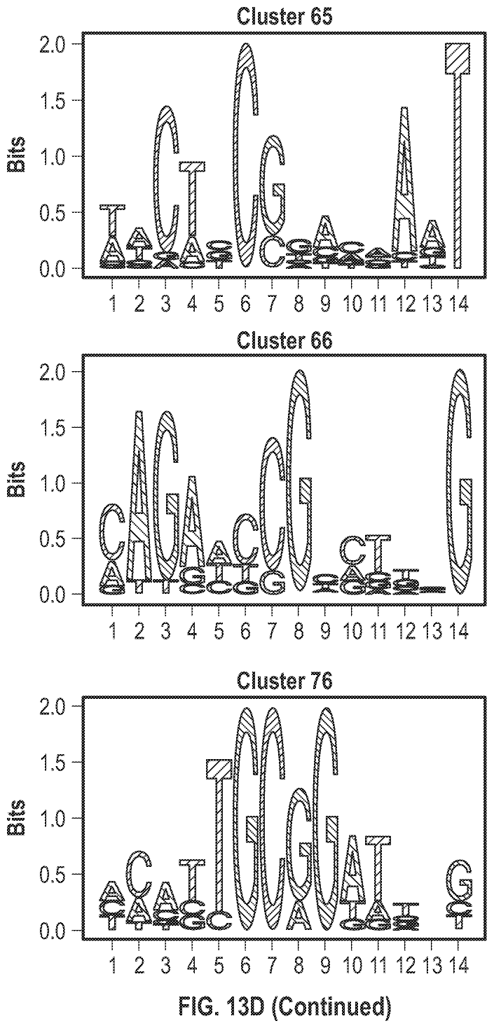

FIG. 13 and Tables 2 and 3 show results of analysis for seven selected candidate RdDM target loci: TARGET OF RAPAMYCIN (TOR), a regulator of cell growth, SPLAYED (SYD), a chromatin remodeling component, UBIQUITIN-SPECIFIC PROTEASE 26 (UBP26), required for heterochromatin silencing, NUCLEAR RNA POLYMERASE D1A (NRPD1), the largest subunit of RNA polymerase IV, RNA POLYMERASE II LARGE SUBUNIT (NRPB1) involved in transcription, SU (VAR) 3-9-RELATED PROTEIN 5 (SUVR5), a gene involved in H3K9me2 deposition and gene silencing, and UP-FRAMESHIFT1 (UPF1), a gene involved in RNA interference and splicing.

TABLE 2
Summary data for gene methylation within motif cluster 11*

| Gene ID | TOR | TOR | NRP1D | SUVR5 | SYD | SYD | UBP26 | UBP26 | UBP26 | NRPB1 | NRPB1 | UPF1 | UPF1 |
|---|---|---|---|---|---|---|---|---|---|---|---|---|---|
| state 1 | +! | − | − | +! | +! | − | − | +! | − | +! | − | + | − |
| state 2 | − | + | + | − | − | − | + | − | − | − | − | − | − |
| state 3 | +! | +! | − | +! | − | − | − | + | − | − | − | +! | − |
| state 4 | − | − | − | − | + | + | − | − | + | − | + | − | + |
| | | | | | (3)** | (3) | | | (3) | | (1) | | (3) |

*+ indicates presence of DMPs in at least two samples, − indicates absence of DMPs, ! indicates dcl2, dcl3, dcl4 sensitivity.
**number in parentheses refers to number of epi-lines displaying DMPs in at least 2 samples.

TABLE 3
Summary data for gene methylation within motif cluster 18*

| Gene ID | TOR | NRPD1 | NRPD1 | SUVR5 | SYD | SYD | UBP26 | NRPB1 | UPF1 | UPF1 |
|---|---|---|---|---|---|---|---|---|---|---|
| state 1 | +! | − | − | +! | − | − | − | +! | +! | +! |
| state 2 | − | − | − | + | − | − | − | + | − | − |
| state 3 | − | − | − | − | + | − | +! | − | +! | +! |
| state 4 | − | + | + | + | + | + | + | − | − | − |
| | | (4) | (1) | (4) | (1) | (1) | (3) | | | |

*+ indicates presence of DMPs in at least two samples, − indicates absence of DMPs, ! indicates dcl2, dcl3, dcl4 sensitivity.
** number in parentheses refers to number of epi-lines displaying DMPs in at least 2 samples.

Differential methylation analysis within the seven loci revealed evidence of state-specific repatterning (Tables 2 and 3). Changes in methylation at each locus were associated with identifiable sequence motifs that spanned approximately 14 nucleotides. Two sample composite motifs are shown in FIG. 13A, with examples of UBP26 and UPF1 state-specific methylation repatterning within cluster 11 shown in FIGS. 13B and 13C. Systematic analysis of the 67 loci revealed similar state-specific changes. The DMPs were assayed individually, although multiple DMPs could be detected within a single identified motif domain. DMPs were discriminated among replicated datasets, with several sites shown to be dcl2/dcl3/dcl4-sensitive in state 1 and state 3 (FIG. 13C; Tables 2 and 3). It was feasible to test dcl2/dcl3/dcl4 sensitivity in only the two states that encompass the msh1 rootstock. Tables 2 and 3 summarize scoring outcomes for cluster 11 and 18 motifs within the seven selected genes.

Identified DMPs did not show an obvious pattern of exon, intron or junction localization, and each gene contained multiple motif sites. Evaluation of cluster motifs that encompass DMPs within the 67 msh1 core hub loci revealed, in many cases, evidence of high-order dependencies. Multiple sequence alignment (MSA) of a given DNA motif can reveal a dependence relationship between two nucleotides located at different positions within the motif, reflected in their frequencies of simultaneous occurrence. First-order dependence refers to adjacent nucleotides, typically found in CG methylation context, second-order to nucleotides spaced two nucleotides apart, and high-order to nucleotides with intervening distance of more than two nucleotides. The relationships derive from the study of Markov dependence in DNA sequences, the basis for application of hidden Markov modeling of motif findings.

For the motifs identified, individual consensus nucleotides were evident at variable distance from the target cytosine, which is nucleotide 7 (on the plus or minus strand) within each motif. For example, the motif from cluster 65 showed invariant T at position 14 and a consensus A at position 12, while the motif from cluster 66 showed invariant G at position 14 and an AG pair consensus at positions 2 and 3, respectively (FIG. 13D). In motif 76, only G resides at positions 6 and 9 with consensus T at position 5, while motifs 82 and 86 showed consensus or invariant T at position 2. These observations support the hypothesis that target methylation sites within these genes are characterized by invariant or nearly invariant sequence features.

Small regions (14 bp) encompassing DMPs in at least three samples were identified and considered as DNA methylation motif candidates in the 67 identified msh1 core hub genes. A distance matrix was estimated on the set of selected regions using function dist.dna from ape R package (version 5.5). Hierarchical cluster analysis on the set of selected regions (using the previous estimated distance matrix) was accomplished with function hclust from stats R package (version 4.1.1) and grouped to 100 clusters. UPGMA approach was applied as agglomeration algorithm. Clusters with fewer than 10 regions where discarded. A DNA multiple sequence alignment on each cluster of sequences was accomplished with MUSCLE algorithm implemented on Bioconductor R package muscle (version 3.14). The motifs presented in FIG. 13A resulted from the specific analysis of genes TOR, SYD, NRPB1, NRPD1, UBP26, SUVR5, and UPF1.

### Example 2: Detection of Signal Peaks

A rapid way to discover DNA sequence features associated with methylation is proceeding with the location of methylation sites across the samples. This approach relies on evidence that methylation takes places at specific, nonrandom sites (a sort of DNA sequence motif).

```
at_sgnl_peaks <- signal_peaks(
  signs = at_signal,
  seq = at_gene_seq,
  cutpoint = 3L,
  upstream = 4,
  downstream = 8L,
  extend = NULL,
  returns = “all”,
  verbose = TRUE)
```

These data are included in the package:

data(at_sgnl_peaks)
at_sgnl_peaks

| | |
|---|---|
| #> | SignalPeakList object of length 7: |
| #> | $AT1G50030 |
| #> | SignalPeak object with 45 ranges and 2 metadata columns: |
| #> | seqnames ranges strand \| seq |
| #> | <Rle> <IRanges> <Rle> \| <character> |
| #> | [1] 1 18523131-18523145 * \| ATCAGCGGGATCTTC |
| #> | [2] 1 18523319-18523333 * \| CGTTTTCGCAGGTTG |
| #> | [3] 1 18523320-18523334 * \| GTTTTCGCAGGTTGA |
| #> | [4] 1 18524813-18524827 * \| AAGTCACGGCACAGC |
| #> | [5] 1 18524814-18524828 * \| AGTCACGGCACAGCA |
| #> | ... ... ... ... . ... |
| #> | [41] 1 18536247-18536261 * \| CATCACGAAGCATGT |
| #> | [42] 1 18537441-18537455 * \| ACTTACCGTTAAATA |
| #> | [43] 1 18537442-18537456 * \| CTTACCGTTAAATAG |
| #> | [44] 1 18537692-18537706 * \| ATTTTCGTCGTCAAG |
| #> | [45] 1 18538007-18538021 * \| AACATTCGATAGTAC |
| #> | encoding |
| #> | <character> |
| #> | [1] 00000101100001001111.. |
| #> | [2] 01101000100100100111.. |
| #> | [3] 01000100100100111111.. |
| #> | [4] 00000001000101100011.. |
| #> | [5] 00001000101100011111.. |
| #> | ... ... |
| #> | [41] 01100000101100011111.. |
| #> | [42] 00001100100100001111.. |
| #> | [43] 01100100100001111111.. |
| #> | [44] 00000100100100101111.. |
| #> | [45] 00000001100000100111.. |
| #> | ------- |
| #> | seqinfo: 1 sequence from an unspecified genome; no seqlengths |
| #> | |
| #> | ... |
| #> | <6 more elements> |

Function signal peaks provides a potential DNA sequence motif with coordinates centered in the methylation peak covering 3-up and 4-down bp around the signal. It was requested that the methylation signal be present in at least three samples (cutpoint=3 L).

### Clustering Potential DNA Sequence Motifs

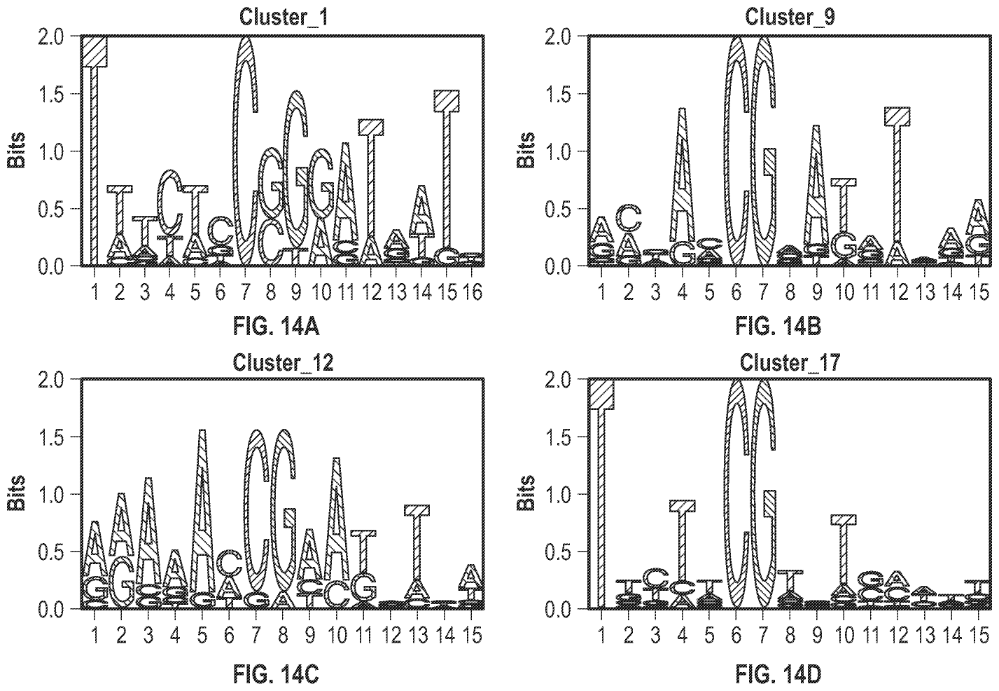

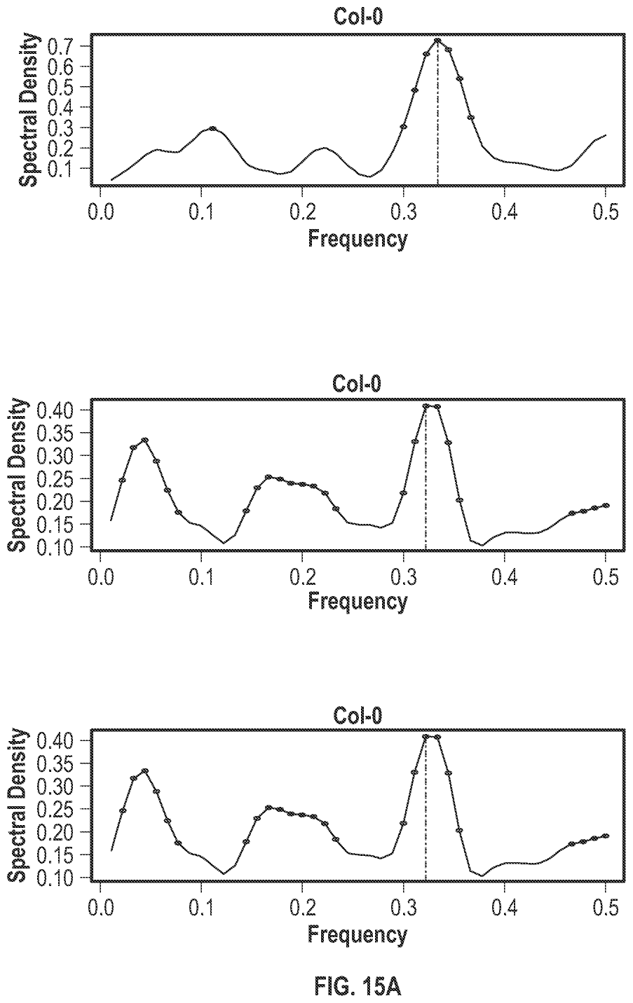

DNA multiple sequence alignment MUSCLE and hierarchical clustering are applied to identify clusters of DNA sequence motifs (FIGS. 14A-14D).

```
at_seq_motif <− seq_clustering(sgn = at_sgnl_peaks, gapOpen = −420,
  min.seq.clust = 10, num.clusters = 21,
  select.aln = TRUE)
m = SeqLogo( aln = at_seq_motif, output = “fm”, plots = F)
ggseqlogo(m, ncol = 2)
```

The cluster motifs that encompass DMPs on gene AT1G50030 (TOR) revealed evidence of high-order dependencies. That is to say, in a multiple sequence alignment (MSA) of a given DNA motif, a base Y at a given site k depends on base X at the preceding site j if high frequencies of bases X and Y are simultaneously observed in the MSA. In particular, if k−j=1, then k is a first order dependence typically found in CG methylation context; if k−j=2, k is a second order dependence, and when k−j>2, a k is a high order dependence.

For these motifs, individual consensus nucleotides were evident at variable distance from the target cytosine, which is nucleotide 7 (on the ‘+’ or the ‘−’ strand) within each motif. For example, the motif from cluster 1 (FIG. 14A) showed only T at position 1 (a maximum score of 2 bit), a consensus G at position 9, Ts at positions 12 and 15 (about 1.5 bit), while motif 17 (FIG. 14D) showed only T at that position 1. In motif 12 (FIG. 14C), a notable consensus of A is found at positions 5 and 10, at the level of methylated C at position 7.

To obtain the DNA sequence motifs by gene:

at_motif <− motifByRegion(sgnl = at_sgnl_peaks, aln = at_seq_motif)
at_motif <− summaryBymotif(at_motif)
at_motif

| | |
|---|---|
| #> | GRanges object with 50 ranges and 8 metadata columns: |
| #> | seqnames ranges strand \| seq encoding |
| #> | <Rle> <IRanges> <Rle> \| <character> <character> |
| #> | 1 1 18523131-18523145 − \| ATCAGCGGGATCTTC 00000101100001001111.. |
| #> | 2 1 18530467-18530481 + \| CTCTCCCGACAAATG 01100101100101101111.. |
| #> | 3 1 18534235-18534249 + \| TTTCTCCGGAATAAT 00100100101100101111.. |
| #> | 4 1 18534236-18534250 − \| TTCTCCGGAATAATG 00100101100101111111.. |
| #> | 12 1 23358046-23358060 + \| TGTCCCCGGGAGATC 00101000101101101111.. |
| #> | .. ... ... ... . ... ... |
| #> | 34 3 18381720-18381734 − \| CGTAACGCATCTGAA 01101000100000011111.. |
| #> | 35 3 18384965-18384979 − \| GAGATCGTATGTCAA 01000001000000111111.. |
| #> | 36 3 18386255-18386269 − \| ACCAGCGGGTGTAGA 00001101100001011111.. |
| #> | 43 4 16964793-16964807 − \| ACTGCCGGATAAGAT 00001100101001111111.. |
| #> | 44 4 16966000-16966014 − \| GATACCGAAGATCTA 01000000100001111111.. |
| #> | consensus inf_content seq_score aln_score cluster gene_id |
| #> | <character> <numeric> <numeric> <numeric> <character> <character> |
| #> | 1 TTTCTCCGGGATAATT 15.7146 −1.4 2.87924 cluster_1 AT1G50030 |
| #> | 2 TTTCTCCGGGATAATT 15.7146 −1.4 2.87924 cluster_1 AT1G50030 |
| #> | 3 TTTCTCCGGGATAATT 15.7146 −0.4 2.87924 cluster_1 AT1G50030 |
| #> | 4 TTTCTCCGGGATAATT 15.7146 −0.6 2.87924 cluster_1 AT1G50030 |
| #> | 12 TTTCTCCGGGATAATT 15.7146 −1.0 2.87924 cluster_1 AT1G63020 |
| #> | .. ... ... ... ... ... ... |
| #> | 34 ACTACCGAATATAAA 11.5253 −1.2 2.78777 cluster_9 AT3G49600 |
| #> | 35 ACTACCGAATATAAA 11.5253 −1.4 2.78777 cluster_9 AT3G49600 |
| #> | 36 ACTACCGAATATAAA 11.5253 −1.2 2.78777 cluster_9 AT3G49600 |
| #> | 43 ACTACCGAATATAAA 11.5253 −1.0 2.78777 cluster_9 AT4G35800 |
| #> | 44 ACTACCGAATATAAA 11.5253 −1.0 2.78777 cluster_9 AT4G35800 |
| #> | ------- |
| #> | seqinfo: 5 sequences from an unspecified genome; no seqlengths |

### Applying Digital Signal Processing (DSP)

DSP can be applied to the previously obtained signals. Next, power spectra from signals from chromosome 2 and 3 are computed.

A function that encodes a previous detected binary signal of 0s and 1s from a DNA sequence into a numerical code defined by the user can be applied. Possible encodings can be binary number, real numbers, and complex numbers. The encoding of DNA methylated sequence using complex numbers is also supported with GWFs. Encoding using ordinary real number is supported as well. The basic idea is to encode the physicochemical properties of DNA bases. In this scenario, by applying different DSP tools, periodicities can be searched for and correlations on the encoded signal that target the superposition of methylation and physicochemical signals. Currently, the DSP analysis of complex signal with R is not good. However, the methylation signal can be encoded with GWF and then exported to, e.g., MatLab or Pythom, and to accomplish the DSP analysis there.

Given two objects, one carrying the signal and the other one carrying the DNA sequence, the function will perform the encoding set out by the user. The function can be used to re-code the previous detected binary signal of 0s and 1s from a DNA sequence into numerical code defined by the user. In particular, it is feasible to incorporate information on the physicochemical properties of neighboring DNA bases: the number of hydrogen bonds and the base chemical type. This can be the default used by this function:
1. Methylation status: 1 (methylated) and 0 (unmathylated)
2. Hydrogen bonds: 1 (three hydrogen bonds) and 0 (two hydrogen bonds)
3. Chemical type: 1 (pyrimidine) and 0 (pyrimidine)

So, each base will rise to a binary string:

1. the signal mC, methylated pyrimidine with three hydrogen bonds: 111
2. C, pyrimidine with three hydrogen bonds: 011
3. G, purine with three hydrogen bonds: 010
4. T, pyrimidine with two hydrogen bonds: 001
5. A, purine with two hydrogen bonds: 000
at_enc_signal <− signalEncoding(

```
  signal = at_signal,
  GR = at_gene_seq,
  S = “111”,
  Sc = “110”,
  Cs = “011”,
  Gs = “010”,
  Ts = “001”,
  As = “000”,
  Us = “001”,
  Ns = ‘000’,
  OS = “100”,
  OC = “101”,
  Bs = “C”,
  O = “A”,
  verbose = FALSE)
```

data(at_enc_signal)
at_enc_signal

| | |
|---|---|
| #> | GroupedSignalEncList object of length: 10 |
| #> | names(10): col1 col2 col3 col_1 col_2 dw 1 dw 2 dw 3 dw_1 dw_2 |
| #> | ------- |
| #> | $col1 |
| #> | SignalEncList object of length: 7 |
| #> | names(7): AT1G50030 AT1G63020 AT2G23740 AT2G28290 AT3G49600 AT4G35800 AT5 |

G47010

| | |
|---|---|
| #> | ------- |
| #> | $AT1G50030 |
| #> | SignalEnc object with 17748 ranges and 3 metadata columns: |
| #> | seqnames ranges strand \| signal seq encoding |
| #> | <Rle> <IRanges> <Rle> \| <numeric> <character> <character> |
| #> | [1] 1 18522248 − \| 0 A 000 |
| #> | [2] 1 18522249 − \| 0 A 000 |
| #> | [3] 1 18522250 − \| 0 C 011 |
| #> | [4] 1 18522251 − \| 0 A 000 |
| #> | [5] 1 18522252 − \| 0 A 000 |
| #> | ... ... ... ... . ... ... ... |
| #> | [17744] 1 18539991 − \| 0 T 001 |
| #> | [17745] 1 18539992 − \| 0 T 001 |
| #> | [17746] 1 18539993 − \| 0 T 001 |
| #> | [17747] 1 18539994 − \| 0 T 001 |
| #> | [17748] 1 18539995 − \| 0 T 001 |
| #> | ------- |
| #> | seqinfo: 1 sequence from an unspecified genome; no seqlengths |
| #> | |
| #> | ... |
| #> | <6 more SignalEnc elements> |
| #> | ------- |
| #> | |
| #> | ... |
| #> | <9 more SignalEncList elements |
| #> | ------- |

Next, the signal is partitioned into intervals of non-overlapping windows of 90 bit (30 bp):

at_enc_signal_win <− slidingWindowSignals(

sgns = at_enc_signal,
win.size = 90,
step.size = 90,
seq_names = “from_region_id”,
na.val = 0,
num.cores = 60L,
tasks = 0,
verbose = FALSE)

data(at_enc_signal_win)
at_enc_signal_win

| | |
|---|---|
| #> | GroupedSignalMatrixList object of length: 10 |
| #> | ------- |
| #> | $col1 |
| #> | SignalMatrixList object of length: 7 |
| #> | names(7): AT1G50030 AT1G63020 AT2G23740 AT2G28290 AT3G49600 AT4G35800 AT5 |

G47010

| | |
|---|---|
| #> | ------- |
| #> | $AT1G50030 |
| #> | SignalMatrix with 592 ranges and 90 metadata columns: |
| #> | seqqnames ranges strand \| 1 2 3 ... |
| #> | <Rle> <IRanges> <Rle> \| <numeric> <numeric> <numeric> ... |
| #> | [1] AT1G50030 1-90 + \| 0 0 0 ... |
| #> | [2] AT1G50030 91-180 + \| 0 0 0 ... |
| #> | [3] AT1G50030 181-270 + \| 0 0 0 ... |
| #> | [4] AT1G50030 271-360 + \| 0 0 1 ... |
| #> | [5] AT1G50030 361-450 + \| 0 0 1 ... |
| #> | ... ... ... ... . ... ... ... ... |
| #> | [558] AT1G50030 52831-52920 + \| 0 1 1 ... |
| #> | [589] AT1G50030 52921-53010 + \| 0 1 0 ... |
| #> | [590] AT1G50030 53011-53100 + \| 0 0 1 ... |
| #> | [591] AT1G50030 53101-53190 + \| 0 0 1 ... |
| #> | [592] AT1G50030 53191-53280 + \| 0 1 1 ... |
| #> | 88 89 90 |
| #> | <numeric> <numeric> <numeric> |
| #> | [1] 0 1 1 |
| #> | [2] 0 0 0 |
| #> | [3] 0 0 0 |
| #> | [4] 0 0 0 |
| #> | [5] 0 0 1 |
| #> | ... ... ... ... |
| #> | [588] 0 0 1 |
| #> | [589] 0 0 0 |
| #> | [590] 0 1 1 |
| #> | [591] 0 0 0 |
| #> | [592] 0 0 0 |
| #> | ------- |
| #> | seginfo: 1 sequence from an unspecified genome; no seqlengths |
| #> | |
| #> | ... |
| #> | <6 more SignalMatrix elements> |
| #> | ------- |
| #> | |
| #> | ... |
| #> | <9 more SignalMatrixList elements> |
| #> | ------- |

There are 592 potential DNA sequence motifs:

AT1G50030_sgns_ranges <− unlist(as.list(at_enc_signal_win$dw 1$AT1G50030@SeqRanges))
seqlevels(AT1G50030_sgns_ranges) <− “1”
AT1G50030_sgns_ranges

| | |
|---|---|
| #> | GRanges object with 592 ranges and 0 metadata columns: |
| #> | seqnames ranges strand |
| #> | <Rle> <IRanges> <Rle> |
| #> | [1] 1 18522248-18522277 + |
| #> | [2] 1 18522278-18522307 + |
| #> | [3] 1 18522308-18522337 + |
| #> | [4] 1 18522338-18522367 + |
| #> | [5] 1 18522368-18522397 + |
| #> | ... ... ... ... |
| #> | [588] 1 18539858-18539887 + |
| #> | [589] 1 18539888-18539917 + |
| #> | [590] 1 18539918-18539947 + |
| #> | [591] 1 18539948-18539977 + |
| #> | [592] 1 18539978-18539995 + |
| #> | ------- |
| #> | seqinfo: 1 sequence from an unspecified genome; no seqlengths |

The overlaps can be searched between the motifs and the 30-bp wide sliding windows.

hits <− findOverlaps(at_sgnl_peaks$AT1G50030, AT1G50030_sgns_ranges,

ignore.strand = TRUE,
type = “within”)

AT1G50030_sgns <− at_enc_signal_win$dw 1$AT1G50030[ subjectHits(hits) ]
AT1G50030_sgns

| | |
|---|---|
| #> | SignalMatrix with 20 ranges and 90 metadata columns: |
| #> | seqnames ranges strand \| 1 2 3 ... 88 |
| #> | <Rle> <IRanges> <Rle> \| <numeric> <numeric> <numeric> ... <numeric> |
| #> | [1] AT1G50030 2611-2700 + \| 0 1 0 ... 0 |
| #> | [2] AT1G50030 7651-7740 + \| 0 1 0 ... 0 |
| #> | [3] AT1G50030 9541-9630 + \| 0 1 0 ... 0 |
| #> | [4] AT1G50030 9541-9630 + \| 0 1 0 ... 0 |
| #> | [5] AT1G50030 19261-19350 + \| 0 1 0 ... 0 |
| #> | ... ... ... ... . ... ... ... ... ... |
| #> | [16] AT1G50030 41581-41670 + \| 1 1 1 ... 0 |
| #> | [17] AT1G50030 41581-41670 + \| 1 1 1 ... 0 |
| #> | [18] AT1G50030 45541-45630 + \| 0 1 0 ... 0 |
| #> | [19] AT1G50030 45541-45630 + \| 0 1 0 ... 0 |
| #> | [20] AT1G50030 47251-47340 + \| 0 1 0 ... 0 |
| #> | 89 90 |
| #> | <numeric> <numeric> |
| #> | [1] 0 1 |
| #> | [2] 1 1 |
| #> | [3] 1 1 |
| #> | [4] 1 1 |
| #> | [5] 0 1 |
| #> | ... ... ... |
| #> | [16] 1 0 |
| #> | [17] 1 0 |
| #> | [18] 0 1 |
| #> | [19] 0 1 |
| #> | [20] 0 0 |
| #> | ------- |
| #> | seqinfo: 1 sequence from an unspecified genome; no seqlengths |

That is, 20 genomic word frameworks from 592 are fully within 30-bp regions on gene AT1G50030. However, only 3 significant motifs are covered by this region in the dwarf sample ‘dw2’.

AT1G50030_motif <− at_motif[ at_motif$gene_id == “AT1G50030” ]
hits <− findOverlaps(AT1G50030_motif, AT1G50030_sgns_ranges,

ignore.strand = TRUE,
type = “within”)

AT1G50030_motif <− AT1G50030_motif[ queryHits(hits ) ]
## Col-0 signal & Dwarf signal
AT1G50030_sgns <− list(

col1 = at_enc_signal_win$col1$AT1G50030[ subjectHits(hits) ],
dw 2 = at_enc_signal_win$dw 2$AT1G50030[ subjectHits(hits) ])

AT1G50030_sgns <− as(AT1G50030_sgns, “SignalMatrixList”)
AT1G50030_sgns

| | |
|---|---|
| #> | SignalMatrixList object of length: 2 |
| #> | names(2): col1 dw 2 |
| #> | ------- |
| #> | $col1 |
| #> | SignalMatrix with 3 ranges and 90 metadata columns: |
| #> | seqnames ranges strand \| 1 2 3 ... 88 |
| #> | <Rle> <IRanges> <Rle> \| <numeric> <numeric> <numeric> ... <numeric> |
| #> | [1] AT1G50030 2611-2700 + \| 0 1 0 ... 0 |
| #> | [2] AT1G50030 38251-38340 + \| 0 1 1 ... 0 |
| #> | [3] AT1G50030 38251-38340 + \| 0 1 1 ... 0 |
| #> | 89 90 |
| #> | <numeric> <numeric> |
| #> | [1] 0 1 |
| #> | [2] 1 0 |
| #> | [3] 1 0 |
| #> | ------- |
| #> | seqinfo: 1 sequence from an unspecified genome; no seqlengths |
| #> | |
| #> | ... |
| #> | <1 more SignalMatrix elements> |
| #> | ------- |

The three motifs are embedded at the beginning of the signal region under scrutiny.

#> * Signal regionCoordinates
unlist(as.list(AT1G50030_sgns$col1@SeqRanges))

| | |
|---|---|
| #> | GRanges object with 3 ranges and 0 metadata columns: |
| #> | seqnames ranges strand |
| #> | <Rle> <IRanges> <Rle> |
| #> | [1] AT1G50030 18523118-18523147 + |
| #> | [2] AT1G50030 18534998-18535027 + |
| #> | [3] AT1G50030 18534998-18535027 + |
| #> | ------- |
| #> | seqinfo: 1 sequence from an unspecified genome; no seqlengths |
| #> | * Motif Coordinates |

cat(“-----------------\n”)

| | |
|---|---|
| #> | ----------------- |

AT1G50030_motif

| | |
|---|---|
| #> | GRanges object with 3 ranges and 8 metadata columns: |
| #> | seqnames ranges strand \| seq encoding |
| #> | <Rle> <IRanges> <Rle> \| <character> <character> |
| #> | 1 1 18523131-18523145 − \| ATCAGCGGGATCTTC 00000101100001001111.. |
| #> | 10 1 18535003-18535017 + \| CAAAAACGCAGTTAT 01100000000000000011.. |
| #> | 7 1 18535004-18535018 − \| AAAAACGCAGTTATG 00000000000000011111.. |
| #> | consensus inf_content seq_score aln_score cluster gene_id |
| #> | <character> <numeric> <numeric> <numeric> <character> <character> |
| #> | 1 TTTCTCCGGGATAATT 15.7146 −1.40000 2.87924 cluster_1 AT1G50030 |
| #> | 10 AAAAACCGAATATCA 12.6939 −1.13333 2.85302 cluster_12 AT1G50030 |
| #> | 7 ACTACCGAATATAAA 11.5253 −1.53333 2.78777 cluster_9 AT1G50030 |
| #> | ------- |
| #> | seqinfo: 5 sequences from an unspecified genome; no seqlengths |

The sequences from cluster 12 & 9 (FIGS. 14B, 14C) differ only on base shifting (3 bit).

##
011000000000000000111110011000010001001000001
##
000000000000000111110011000010001001000001010

### Power Spectral Analysis on Experimental Datasets

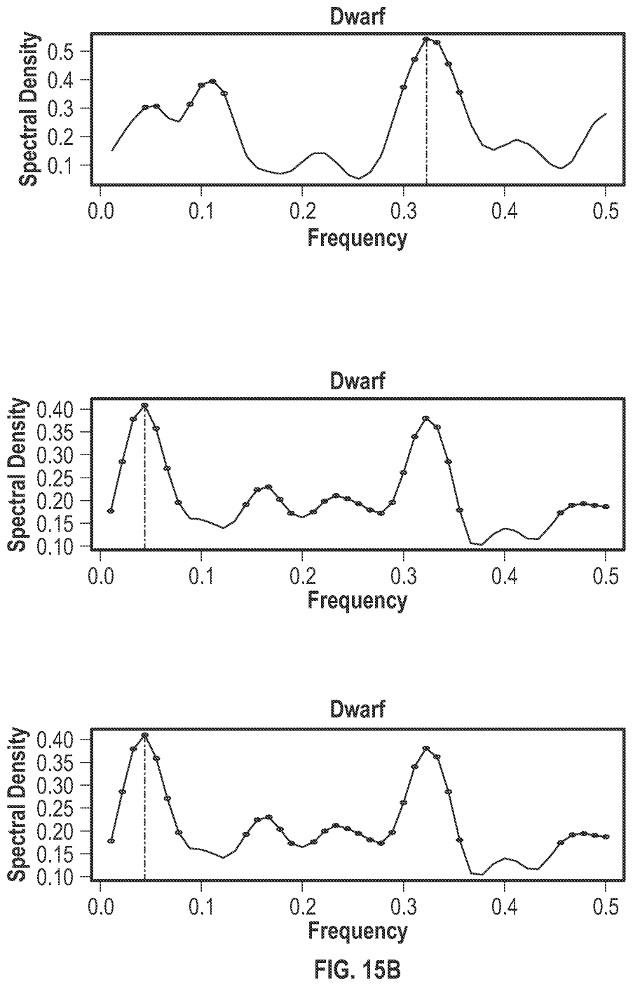

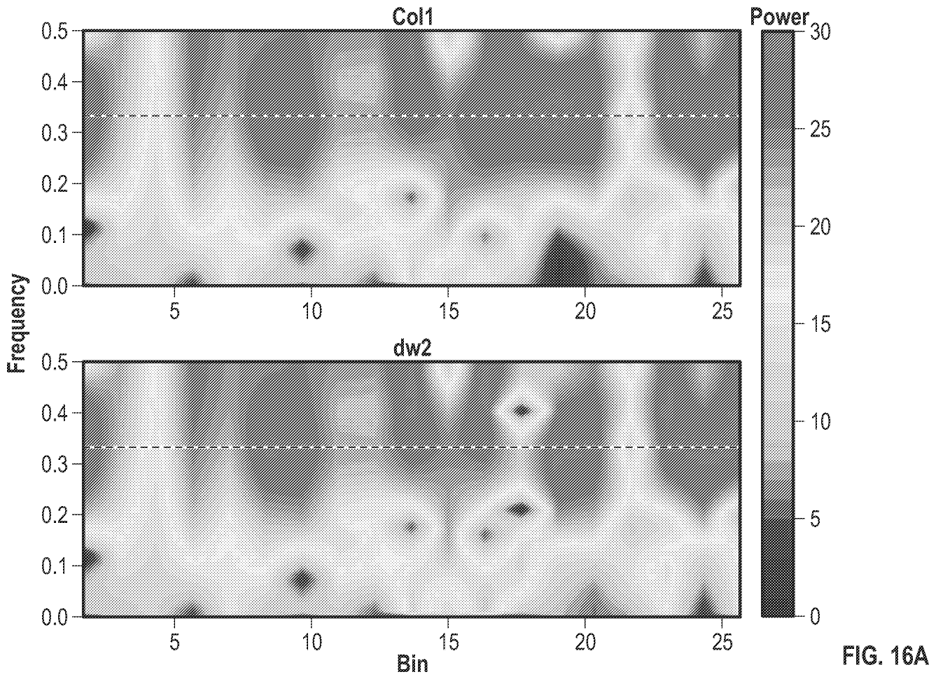

The power spectral of the binary signal can be obtained from the SignalMatrix-class objects using function plot power_spectral (FIG. 15). Dots help to visually identify the peaks where spectral differences between the control (wt) (FIG. 15A) and the treatment (msh1 mutant, dwarf phenotype) (FIG. 15B) were found.

```
plot_power_spectral(x = AT1G50030_sgns,
  main.lab = c(“Col-0”, “Dwarf”),
  range.ind = 1:3)
```

The peak at ⅓ (0.33) indicates the regions under scrutiny are protein coding regions (FIG. 15A). Methylation may or may not affect the period ⅓. On protein coding regions, peaks other than the one at ⅓ results from, in the current case, periodicities originating from DNA base physicochemical properties encoded in the binary vectors representing the base sequences.

### The Spectrogram

A spectrogram is a visual representation of the spectrum of frequencies of a signal as it varies with time. In our case, the axis ‘time’ will be represented by bit or by single DNA base positions. The spectrogram can be obtained from any encoded signal region using function ‘spectrogram’ from the R package phonTools.

To prepare the methylation signal as a numerical signal:

AT1G50030_sgnl = as(AT1G50030_sgns, “GroupedEncNumSignalList”)
AT1G50030_sgnl

| | |
|---|---|
| #> | GroupedEncNumSignalList object of length: 2 |
| #> | names(2): col1 dw 2 |
| #> | ------- |
| #> | $col1 |
| #> | encNumSignalList object of length: 3 |
| #> | names(3): AT1G50030.1 AT1G50030.2 AT1G50030.3 |
| #> | ------- |
| #> | $AT1G50030.1 |
| #> | encNumSignal object of length: 90 |
| #> | [1] 1=0 2=1 3=0 4=0 5=0 6=0 7=0 8=1 ... 83=1 84=1 85=0 86=0 87=0 88=0 89=0 90=1 |
| #> | Element genomic coordinates in the slot object@SeqRanges |
| #> | Element encoding coordinates in the slot object@EncRanges |
| #> | ------- |
| #> | |
| #> | ... |
| #> | <2 more encNumSignal elements> |
| #> | ------- |
| #> | |
| #> | ... |
| #> | <1 more encNumSignalList elements> |
| #> | ------- |

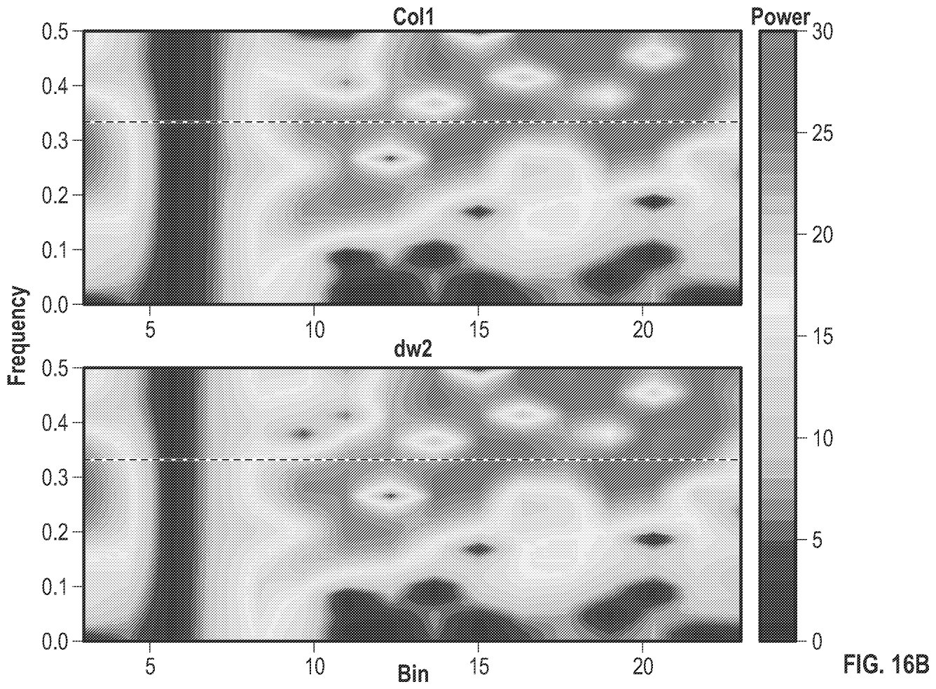

Next, the spectrogram for the same gene from the control (FIG. 16A, top panel) and from treatment (FIG. 16A, bottom panel) is easily computed:

motifs <− AT1G50030_motif$seq
motif.num <− 1
at_spg <− spectrograms(X = AT1G50030_sgnl,

sample_names = c(“col1”, “dw 2”),
region.id = motif.num,
fs = 3000,
windowlength = 3,
timestep = −20,
padding = 10,
preemphasisf = 50,
maxfreq = 5000,
abs.freq = FALSE,
dynamicrange = 30,
nlevels = dynamicrange,
maintitle = paste0(c(“col1”, “dw 2”), “ ”,
AT1G50030_motif$seq[motif.num]),
key.title = “Power”,
window = ‘hann’,
windowparameter = 4,
quality = TRUE,
return.spect = TRUE)

Methylation signal breaks down the power spectrum energy around the periodicity at about the ⅓ frequency (dashed line in FIG. 16A) and bin 16. In the third region it was found:

motif.num <− 3
at_spg <− spectrograms(X = AT1G50030_sgnl,

sample_names = c(“col1”, “dw 2”),
region.id = motif.num,
fs = 3000,
windowlength = 6,
timestep = −20,
padding = 12,
preemphasisf = 50,
maxfreq = 5000,
abs.freq = FALSE,
dynamicrange = 30,
nlevels = dynamicrange,
maintitle = paste0(c(“col1”, “dw 2”), “ ”,
AT1G50030_motif$seq[motif.num]),
key.title = “Power”,
window = ‘hann’,
windowparameter = 4,
quality = TRUE,
return.spect = TRUE)

In this case, the methylation effect lies around bin 6-10, and the energy power is shifted down the ⅓ frequency (FIG. 16B). Essentially, the effect of methylation on the signal power and periodicity depends on the DNA sequence context. This is an expected result that has been ignored by traditional methylation analyses to date.

### Wavelet Power Spectrum

The wavelet coefficients yield information on the correlation between the wavelet (at a certain scale) and the data array (at a particular location). A larger positive amplitude implies a higher positive correlation, while a large negative amplitude implies a high negative correlation.

Wavelet Power Spectrum provides a useful way to determine the distribution of energy within the data array. By looking in the plot for regions within the Wavelet Power Spectrum (WPS) of large power, one can determine which features of the signal are important and which can be ignored. Here, the term “energy” is not arbitrary but is borrowed from applications in human-built communication systems. The level of energy represented in the WPS is proportional to the energy dissipated in the transmission of a binary signal of the same size to a given receiver through a human-built communication grid.

Moreover, with the advance of information theory and its application to biomolecular processes, it is well known that to accomplish a single methylation change, every methyltransferase/demethylase must dissipate a minimal energy to process the information associated with the change. This energy is determined by Landauer's principle, according to which, a molecular machine must dissipate a minimum energy of ε=k_{B}T ln2 (about 3×10-21 Joules per bit at room temperature) at each step in the genetic logic operations including proofreading.

### Wavelet Power Spectral Analysis on Experimental Datasets

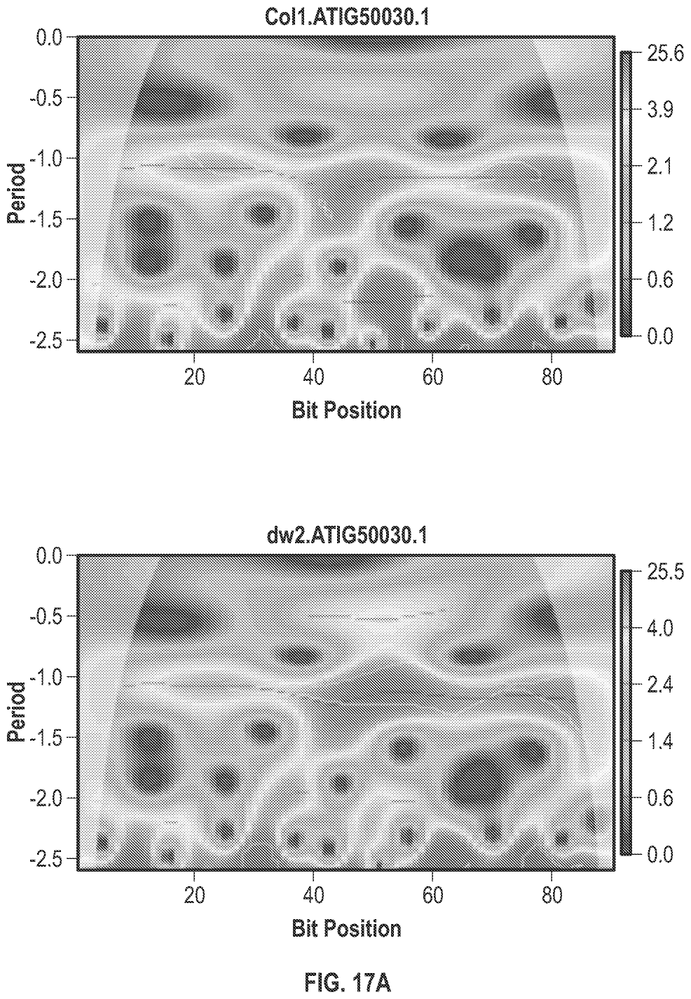

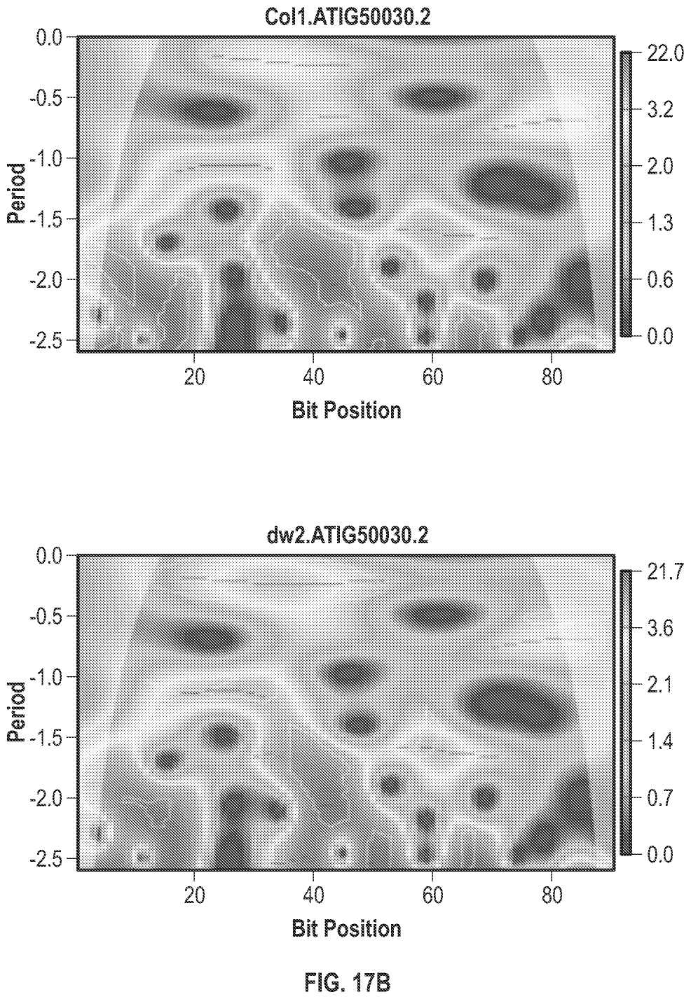

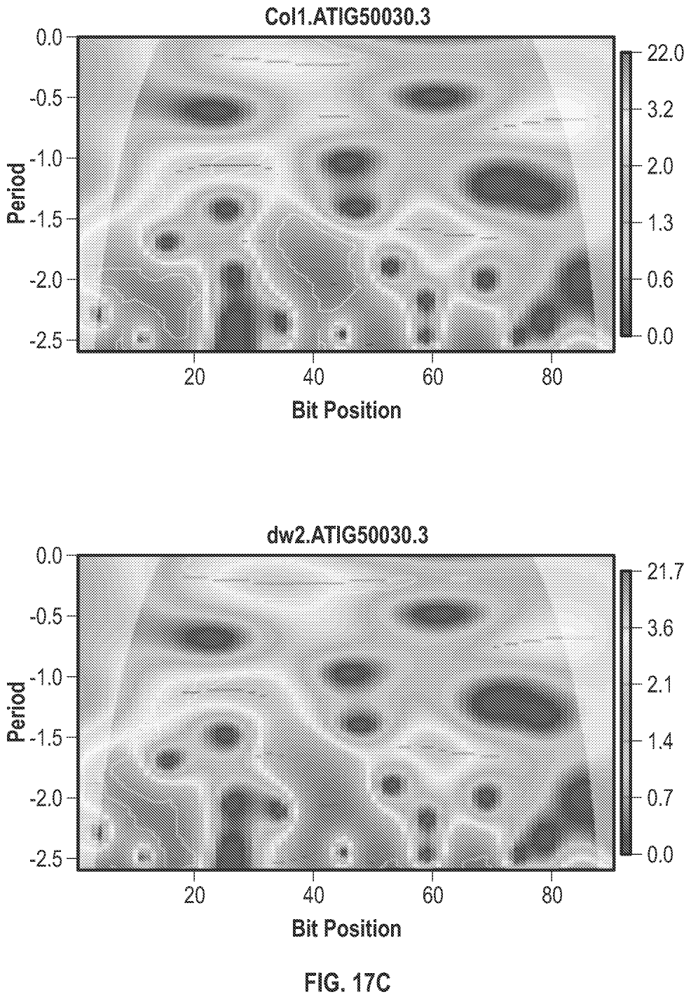

Wavelet Power Spectral analysis of the previously estimated at_signal_diff dataset (FIG. 17) is accomplished by:

```
# data(at_signal_diff)
bin_sgn = as.matrix(AT1G50030_sgnl)
idx <− matrix(1:6, ncol = 2)
idx <− unlist(split(idx, 1:nrow(idx)))
bin_sgn = unlist(bin_sgn, recursive = FALSE)[ idx ]
upperPeriod = floor(length(bin_sgn)/3)/2
at_wav <− plot_ts_wavelet(sp = bin_sgn,
  loess.span = 0.5,
  dt = 1/20, dj = 1/40,
  lowerPeriod = 1/6,
  upperPeriod = upperPeriod,
  yline = 1.8, return.wt = TRUE)
## bit differences
c(diff = mapply(function(x,y) sum(x != y), bin_sgn[c(1,3,5)], bin_sgn[c(2,4,6)] ))
```

Some methylation motifs carry the same methylation status in the same regions from both groups, Col-0 and msh1 Dwarf. This is the case of regions: AT1G50030.3 (FIG. 17C) and AT1G50030.6. The alteration observed in the wavelet power (WPS) spectrum lead to base where the signal is located.

In the case of AT1G50030.1 (FIG. 17A), the WPS indicates that the alteration takes place at 60 bit, which implies the base at position 20=60/3. The table below confirms our estimation. A methylated cytosine is located at base 20 in the negative strand from Col-0. The reported base in the positive strand is G, which is encoded by the binary string 110 when the complementary base C is methylated. The sample base position is not methylated in msh1 Dwarf-2 (G: 010).

col_seq = encoding2seq(encsignal = bin_sgn$col1.AT1G50030.1,

codeword size = 3L,
verbose = FALSE)

dw_seq = encoding2seq(encsignal = bin_sgn$dw 2.AT1G50030.1,

codeword size = 3L,
verbose = FALSE)

data.frame(col_seq, dw_enc = dw_seq$encoding)

| | |
|---|---|
| #> | seqnames start end width strand signal seq encoding dw_enc |
| #> | 1 1 1 1 1 + 0 G 010 010 |
| #> | 2 1 2 2 1 + 0 A 000 000 |
| #> | 3 1 3 3 1 + 0 G 010 010 |
| #> | 4 1 4 4 1 + 0 G 010 010 |
| #> | 5 1 5 5 1 1 0 A 000 000 |
| #> | 6 1 6 6 1 + 0 A 000 000 |
| #> | 7 1 7 7 1 + 0 G 010 010 |
| #> | 8 1 8 8 1 + 0 A 000 000 |
| #> | 9 1 9 9 1 + 0 T 001 001 |
| #> | 10 1 10 10 1 + 0 C 011 011 |
| #> | 11 1 11 11 1 + 0 T 001 001 |
| #> | 12 1 12 12 1 + 0 A 000 000 |
| #> | 13 1 13 13 1 + 0 T 001 001 |
| #> | 14 1 14 14 1 + 0 A 000 000 |
| #> | 15 1 15 15 1 + 0 T 001 001 |
| #> | 16 1 16 16 1 + 0 C 011 011 |
| #> | 17 1 17 17 1 + 0 A 000 000 |
| #> | 18 1 18 18 1 + 0 G 010 010 |
| #> | 19 1 19 19 1 + 0 C 011 111 |
| #> | 20 1 20 20 1 + 0 G 010 110 |
| #> | 21 1 21 21 1 + 0 G 010 010 |
| #> | 22 1 22 22 1 + 0 G 010 010 |
| #> | 23 1 23 23 1 + 0 A 000 000 |
| #> | 24 1 24 24 1 + 0 T 001 001 |
| #> | 25 1 25 25 1 + 0 C 011 011 |
| #> | 26 1 26 26 1 + 0 T 001 001 |
| #> | 27 1 27 27 1 + 0 T 001 001 |
| #> | 28 1 28 28 1 + 0 C 011 011 |
| #> | 29 1 29 29 1 + 0 A 000 000 |
| #> | 30 1 30 30 1 + 0 T 001 001 |

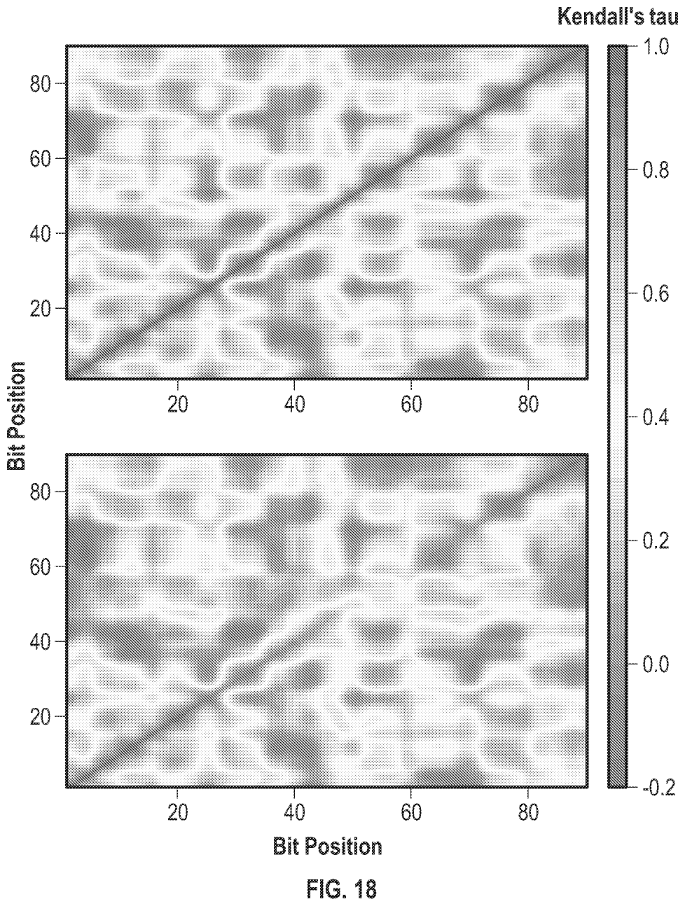

In the same way the correlogram can be computed based on WPS (FIG. 18):

| | |
|---|---|
| A) | X = cor_spg(spg = at_wav, |
| | sample.index = 1, |
| | indv.index = c(1,1), |
| | bp = NULL, |
| | interval = c(1,1), |
| | output = “cor.coef”, |
| | per.col = T, |
| | verbose = F) |

Y = cor_spg(spg = at_wav,

```
  sample.index = 1:2,
  indv.index = c(1,1),
  bp = NULL,
  interval = c(1,1),
  output = “cor.coef”,
  per.col = T,
  verbose = F)
```

labs <− paste0(c(“col1”, “dw 2”), “ ”,
AT1G50030_motif$seq[motif.num])
correlogram(X,Y,

maintitle = c(paste0(labs[1], “ vs ”, labs[1]),
paste0(labs[1], “ vs ”, labs[2])),
axis.lab = c(“Bit position”, “Bit position”),
key.title = “Kendall's tau”,
yline = 1, cex.title = 1.7)

In addition to the expected correlation break at region 55-60 bit, the correlation between energy spectrum at regions 30-35 bit and 55-60 bit is lost (FIG. 18).

### Example 3: Human Data Sets

The analysis of the methylation signal accomplished on 81 genes associated to human neural system development.

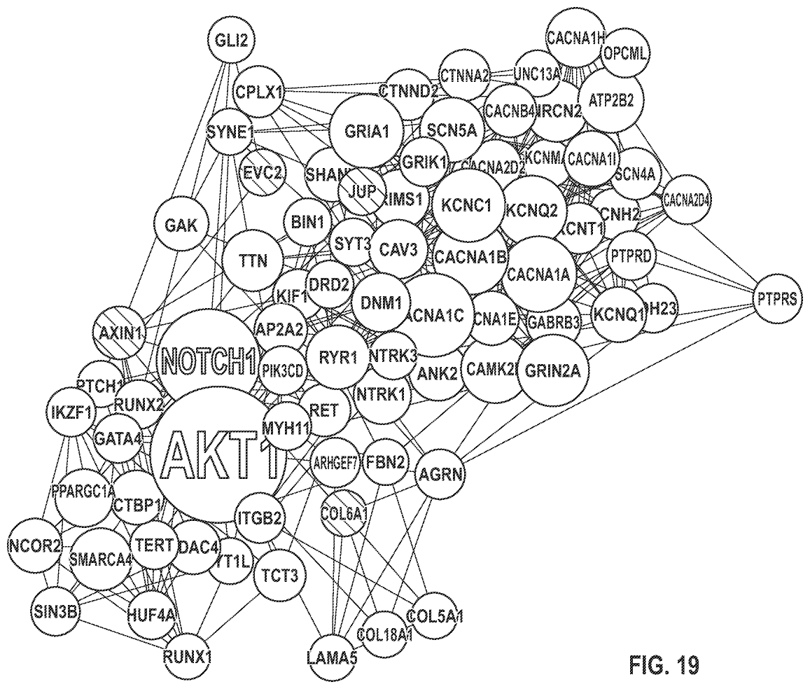

FIG. 19 is the sub-network of 81 DMG-hubs. A PPI network was built on the set of 751 DMGs identified with principal component analysis were analyzed with STRING Cytoscape App. Circles free of crosshatching are highlighted DMGs involved in the biological processes that include nervous system, nervous system development, synapse, neuron projection, ion transport, central nervous system disease, axon guidance, neurogenesis, ion/cation biding, ion/cation transmembrane transport, voltage-gated channel and TRP channels.

The whole set of DMGs derives from 751 DMGs, which were selected according to their contribution to patient classification into two groups: “typical” and “autism”. Concretely, the 751 DMGs contribute with more than 1% of the total variance to the main principal component from a PCA. These DMGs were analyzed with STRING Cytoscape App and the main sub-network of hub was identified applying K-means clustering approach using network centrality indicators as variables. All the DMGs were obtained with methylation analyses.

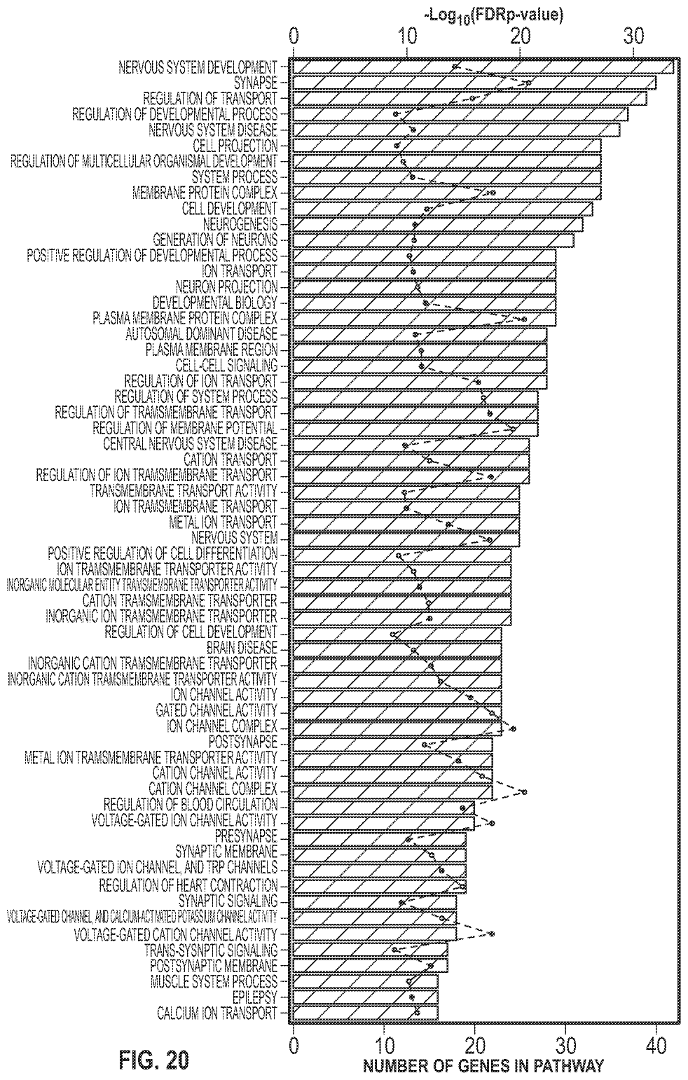

FIG. 20 is the network enrichment analysis identified in the main network of DMG-hub from FIG. 19. The methylation motifs identified in 81 DMG members of the main sub-network of hubs derived from a network of genes associated with autism can be achieved by methods described herein.

From the foregoing, it can be seen that the present disclosure accomplishes at least all of the stated objectives.

### Glossary

Unless defined otherwise, all technical and scientific terms used above have the same meaning as commonly understood by one of ordinary skill in the art to which embodiments of the present disclosure pertain.

The terms “a,” “an,” and “the” include both singular and plural referents.

The term “or” is synonymous with “and/or” and means any one member or combination of members of a particular list.

The terms “invention”, “present invention”, “disclosure”, or “present disclosure” are not intended to refer to any single embodiment of the particular invention but encompass all possible embodiments as described in the specification and the claims.

The term “about” as used herein refer to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variable, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

“Phenotype” refers to the set of observable characteristics of an individual resulting from the interaction of its genotype with the environment.

“Epigenetic” relates to arises from nongenetic influences on gene expression.

An “R package” is an extension to the R statistical programming language. R packages contain code, data, and documentation in a standardized collection format that can be installed by users of R, typically via a centralized software repository such as CRAN.

“Expressivity” is the degree to which a phenotype is expressed by individuals having a particular genotype.

The “scope” of the present invention is defined by the appended claims, along with the full scope of equivalents to which such claims are entitled. The scope of the invention is further qualified as including any possible modification to any of the aspects and/or embodiments disclosed herein which would result in other embodiments, combinations, subcombinations, or the like that would be obvious to those skilled in the art.

What is claimed is:

1. An extension for a general purpose programming language or a statistical programming language, said extension comprising:
algorithm(s) that analyzes methylation signals on stretches of DNA sequences, said DNA sequences being characterized by:
iii. methylation information; and
iv. physicochemical information around each methylated cytosine;
wherein the algorithm(s) include one or more functions that can:
estimate a distance matrix on a set of selected regions of said DNA sequences;
analyze a hierarchical cluster on the set of selected regions;
group the set of selected regions into a specified number of clusters; and
align multiple DNA sequences from the clusters into methylation motifs.

2. The extension of claim 1, wherein the extension is written in the R statistical language.

3. The extension of claim 2, further comprising a digital signal processing (DSP) tool available in another programing language.

4. The extension of claim 3, wherein the another programming language is C++, Python, or MatLab.

5. The extension of claim 3, further comprising:
encoding the DNA sequence and physicochemical properties of DNA base into a numerical or complex signal further comprising the DSP.

6. The extension of claim 5, wherein said encoding is based on a group structure.

7. The extension of claim 6, wherein said group structure is an Abelian group.

8. The extension of claim 1, wherein the set of selected regions is grouped into groups of at least 100 clusters.

9. The extension of claim 1, wherein clusters with less than ten regions are discarded.

10. The extension of claim 1, wherein the multiple DNA sequences are aligned using a multiple sequence comparison by a log-expectation algorithm.

11. The extension of claim 5, further comprising:
exporting the numerical or complex signal into C++, Python, or MatLab.

12. The extension of claim 1, wherein the methylation motifs are further grouped for downstream analysis.

13. The extension of claim 12, wherein the methylation motifs are further grouped using a clustering algorithm.

14. The extension of claim 13, wherein the clustering algorithm is a distance-based k-medoids clustering algorithm.

15. The extension of claim 1, further comprising:
encoding the methylation and physicochemical signals of DNA bases into one binary, numerical, or complex signal.

16. The extension of claim 15, wherein said encoding is based on a group structure.

17. The extension of claim 16, wherein said group structure is an Abelian group.

18. The extension of claim 15, further comprising:
conducting a power spectral analysis on the encoded methylation and physicochemical signals.

19. The extension of claim 18, wherein the power spectral analysis is a wavelet power spectral analysis (WPS).

20. The extension of claim 19, further comprising:
computing a correlogram based on the WPS.

21. A computerized heuristic comprising:
a high order DNA base interdependence with respect to methylated cytosines; and
a base distribution that is statistically nonrandom.

22. The computerized heuristic of claim 21, wherein said high order DNA interdependence and said base distribution result from analysis of at least one hierarchical cluster on a region of a DNA sequence and aligning multiple DNA sequences from the clusters into methylation motifs.

23. A method for analyzing methylation signals on stretches of DNA sequences, said method comprising:
analyzing a hierarchical cluster on regions of the DNA sequences;
grouping a set of selected regions hierarchically into a specified number of clusters;
aligning potential DNA sequence motifs from said clusters; and
applying digital signal processing to the encoded methylation and physicochemical signals.

24. The method of claim 23, wherein the set of selected regions is grouped into groups of at least 100 clusters.

25. The method of claim 23, wherein clusters with less than ten regions are discarded.

26. The method of claim 23, wherein said encoded methylation and physicochemical signals are encoded based on group structure.

27. The method of claim 26, wherein said group structure is an Abelian group.

28. The method of claim 23, wherein the potential DNA sequences are aligned using a multiple sequence comparison by log-expectation algorithm.

29. The method of claim 23, further comprising:
conducting a power spectral analysis on the encoded methylation and physicochemical signals.

30. The method of claim 29, wherein the DSP is applied through use of a genomic-word-framework (GWF) R package.

31. The method of claim 30, wherein the GWF R package includes one or more clustering algorithms.

32. The method of claim 30, further comprising exporting the GWF R package to be analyzed with other DSP tools in a different programming language.

33. The method of claim 32, wherein the different programming language is C++, Python, or MatLab.

34. The method of claim 23, further comprising:
using methylation analyses to identify differentially methylated positions (DMPs) that form part of the methylation signals.

35. The method of claim 23, further comprising:
evaluating departure of each of multiple sequence alignments (MSA) from random Monte Carlo simulated MSAs.

36. The method of claim 23, further comprising:
analyzing genes with epigenetic regulatory functionalities to diagnose or cure a disease.

37. The method of claim 36, wherein the disease is autism or cancer.

38. A computerized heuristic for analyzing genetic data comprising:
(1) a statistic of an information divergence (ID) estimated for each gene carrying at least one differentially methylated position (DMP) on the gene or on a promoter region;
(2) principal component analysis (PCA), wherein the first k-th components carrying 1% or more of the whole sample variance are considered in the downstream analysis;
(3) computation of a correlation matrix carrying the pairwise gene correlation, represented as vectors of PCs;
(4) analysis of the correlation matrix for a network; and
(5) contribution of each gene to the discrimination of phenotypes evaluated in terms of the fraction of a cumulative variance from a whole sample variance carried by the gene.

39. The computerized heuristic of claim 38, wherein the ID is selected from the group consisting of: Hellinger divergence/distance, J divergence, and total variation distance.

40. The computerized heuristic of claim 39, wherein the ID is J divergence.

41. The computerized heuristic of claim 38, wherein the PCA is applied with a function such that genes are represented as k-dimensional vectors of PCs, and further wherein the square of each coordinate carries the vector contribution in terms of variance to the treatment discrimination from the control group.

42. The computerized heuristic of claim 38, wherein the PCA is applied with a pcaLDA function.

43. The computerized heuristic of claim 38, wherein the correlation matrix comprises a weighted correlation network (WCN), wherein the WCN and the network are analyzed, and the network is a Protein to Protein Interaction (PPI) network.

44. The computerized heuristic of claim 43, further comprising a comparison of results from the WCN and the PPI network to identified consistent relationships and epigenetic gene contributions to the phenotypes.

45. The computerized heuristic of claim 38, further comprising a magnitude computed as the Euclidean Norm of the gene represented as a vector of k PCs.

46. The computerized heuristic of claim 38, wherein the network relates to one or more biological processes.

47. A method of reducing computation load, said method comprising:
identifying differentially methylated genes (DMGs) on stretches of DNA sequences using methylation analysis;
integrating said DMGs to gene networks; and
identifying network hubs.

48. The method of claim 47, wherein the network hubs are identified via Protein to Protein Interaction (PPI) network analysis and weighted correlation network (WCN) analysis.

49. The method of claim 48, further comprising comparing results from the WCN and the PPI network to identified consistent relationships and epigenetic gene contributions to the phenotypes.

50. The method of claim 47, wherein the network hubs relate to one or more biological processes.
